(19) United States
(12) Patent Application Publication
(10) Pub. No.: US 2014/0004141 A1
(43) Pub. Date: Jan. 2, 2014

(54) Methods And Compositions Of Protein Antigens For The Diagnosis And Treatment Of Toxoplasma Gondii Infections And Toxoplasmosis

(71) Applicants: David Huw Davies, Long Beach, CA (US); Xiaowu Liang, La Jolla, CA (US); Philip Felgner, Rancho Santa Fe, CA (US)

(72) Inventors: David Huw Davies, Long Beach, CA (US); Xiaowu Liang, La Jolla, CA (US); Philip Felgner, Rancho Santa Fe, CA (US)

(73) Assignee: Immport Therapeutics, Inc., Irvine, CA (US)

(21) Appl. No.: 13/997,603

(22) PCT Filed: Dec. 20, 2011
(86) PCT No.: PCT/US11/66178

§ 371 Date: Sep. 4, 2013

Related U.S. Application Data
(60) Provisional application No. 61/426,902, filed on Dec. 23, 2010.

(51) Int. Cl. C07K 14/45

(52) U.S. Cl. CPC C07K 14/45
USPC 424/191.1; 530/350

(57) ABSTRACT

Contemplated compositions, devices, and methods are drawn to various antigens from the pathogen T. gondii and their use in various diagnostic tests, vaccines, and therapeutic agents. In particularly preferred aspects, the antigens are immunodominant and have quantified and known relative reactivities with respect to sera of a population infected with the pathogen, and/or have a known association with a disease parameter.

This application claims the benefit of priority to U.S. provisional patent application with the Ser. No. 61/426,902, which was filed Dec. 23, 2010, and is incorporated by reference herein.

### FIELD OF THE INVENTION

The field of the invention is compositions and methods related to selected antigens from Toxoplasma gondii, especially as they relate to their use in diagnostic and therapeutic compositions and methods.

### BACKGROUND OF THE INVENTION

Toxoplasmosis is a widespread disease caused by infection with the intracellular protozoan Toxoplasma gondii. This organism has a complex life cycle, involving both primary and secondary hosts. Primary hosts are members of the family Felidae, including domestic cats, which can transmit the organism to humans via T. gondii oocytes that are released in the primary host's feces. The organism also infects a wide variety of secondary hosts in addition to humans, and these can transmit T. gondii to humans via ingestion of tissue cysts that contain tachyzoites. Tenter and Weiss (Tenter, A. M., Heckeroth, A. R. and Weiss, L. M. 2000. “Toxoplasma gondii: from animals to humans”. International Journal for Parasitology. 30:1217-1258) estimate that approximately one-third of the world's population have been exposed to the parasite. Infection begins with an acute phase, which may cause flu-like symptoms, and in some individuals may proceed to a chronic phase in which the organism is held in check by the host's immune system. Although infection with T. gondii can be essentially asymptomatic, infection of immunocompromised individuals can lead to serious illness and death. In addition, infections in pregnant women can lead to severe birth defects.

Assays exist to perform limited analysis of T. gondii, and are typically directed to host antibodies to the organism rather than direct detection of the parasite itself. One example is the Sabin-Feldman Dye Test test, which indirectly tests for the presence of host antibodies to the parasite. This test, however, is used by only a few specialized diagnostic laboratories owing to the requirement for cultured organisms and high levels of technical skill. CA 1206904 describes a delayed hypersensitivity test utilizing scarification of the patient with an antigenic preparation derived from the parasite. U.S. Pat. No. 3,914,400, EP72319B1, and EP328588B1 describe agglutination-based assays for host antibodies to T. gondii. Such assays typically rely on highly skilled individuals to perform the test and manually assess the results. More rapid, user-friendly, and objective ELISAs for T. gondii specific antibodies are also employed for diagnostic purposes, but identification of antibodies to appropriate antigens that provide sensitive and accurate detection of acute and chronic toxoplasmosis remains a challenge. EP353111A1 discloses a T. gondii antigen, P30, with diagnostic utility for the organism. U.S. Pat. No. 6,326,008B1, EP748815B1, EP748816B1, EP751147B1, and EP431541B1 similarly describe specific antigens for use in identifying infection with this organism. JP11225783A and EP2062913B1 disclose specific antigens that are useful for diagnosis of toxoplasmosis and that permit differentiation of acute and chronic infections. Similarly US20030119053A1 discloses specific panels of T. gondii antigens, the host IgG and IgM responses to which can be used to identify acute and chronic infection with the parasite.

Efforts to determine T. gondii antigens that are indicative of either acute or chronic infection have utilized antibodies from infected individuals as specific probes. U.S. Pat. No. 6,326,008B1 and EP301961B1 describe the use of immunoprecipitation with immune sera to identify T. gondii antigens associated with acute and chronic toxoplasmosis. EP2062913B1 describes the use of sera from individuals suffering from acute infections to identify a limited number of plaques carrying antigens generated by phage display of T. gondii cDNA. WO2011084044A1 discloses identifying both host and T. gondii proteins characteristic of individuals with different types of infection by separation using 2D electrophoresis, followed by Western blotting with immune serum. Currently, however, high-throughput proteomic research methodologies that allow the rapid screening of large numbers of potential antigens have not been used to analyze Toxoplasma gondii.

Unfortunately, current testing methods yield only partial useful results, testing performance differs widely, and results are too open to misinterpretation. For example, they may indicate exposure to T. gondii, but not provide information on whether such exposure is current or past. Additionally, T. gondii-specific IgM may persist for up to two years after the original infection date. Finally, most current tests also require complex secondary testing procedures to provide useful diagnostic information. T. gondii infection in immunocompromised individuals provides even more challenges, as their antibody response to the infection may differ significantly from the general population. For example, the concentration of IgG, which is the immunoglobulin species detected in many of the current tests, is often so low in individuals with AIDS that it frequently falls below the limit of detection. Additionally, accurate confirmation is particularly important in cases of suspected acute T. gondii infections during pregnancy as decisions whether to terminate a pregnancy will rest on accurate diagnosis. It should also be noted that it has not been lost on investigators that T. gondii antigens that evoke host immune responses may have therapeutic uses. U.S. Pat. No. 6,902,926B1, EP748816B1, EP751147B1, and EP431541B1 describe identification of T. gondii antigens that may have utility in vaccines directed to the parasite using immune sera.

Consequently, there remains a large, unmet need to provide improved compositions and methods of antigen and antibody detection and monitoring for diagnostic and therapeutic applications related to T. gondii.

### SUMMARY OF THE INVENTION

A proteome-microarray approach was used to profile the antibody response during infection against thousands of different T. gondii proteins with the aim of identifying (1) novel IgG and IgM target antigens that discriminated uninfected from infected cases, (2) IgM target antigens that were high in acute infection but which declined thereafter, and (3) IgG target antigens that were low in acute infection but high in chronic with persisting IgM. To address such aims, protein microarrays were used to screen 1,357 prioritized T. gondii exon products with 106 well-characterized sera from toxoplasmosis cases and controls.

Both well-known and novel antigens were identified that could have not been recognized using conventional methodologies. Surprisingly, not only target antigens of IgG and IgM specifically associated with T. gondii infection were identified, but also select T. gondii antigens that can discriminate between: 1) acutely infected, 2) chronically infected with persistent IgM, and 3) true chronically infected hosts. Target antigens that were identified include: TGME49_{—}000470_{—}1, TGME49_{—}001390_{—}1, TGME49_{—}004130_{—}13, TGME49_{—}005300_{—}6, TGME49_{—}005360_{—}14, TGME49_{—}005740_{—}9, TGME49_{—}012300_{—}5, TGME49_{—}013340_{—}4, TGME49_{—}014610_{—}11, TGME49_{—}014760_{—}2, TGME49_{—}016180_{—}1, TGME49_{—}016380_{—}13, TGME49_{—}016380_{—}5, TGME49_{—}021310_{—}9, TGME49_{—}023540_{—}10, TGME49_{—}023540_{—}5, TGME49_{—}024190_{—}10, TGME49_{—}024920_{—}4, TGME49_{—}025320_{—}5, TGME49_{—}026020_{—}8, TGME49_{—}026110_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044040_{—}8, TGME49_{—}044080_{—}3, TGME49_{—}044280_{—}1, TGME49_{—}045500_{—}3, TGME49_{—}046330_{—}5, TGME49_{—}046340_{—}2, TGME49_{—}047370_{—}4, TGME49_{—}047370_{—}9, TGME49_{—}048200_{—}3, TGME49_{—}048670_{—}5, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057080_{—}9, TGME49_{—}057520_{—}1, TGME49_{—}058390_{—}1, TGME49_{—}058980_{—}1, TGME49_{—}059200_{—}2, TGME49_{—}061740_{—}1, TGME49_{—}062920_{—}5, TGME49_{—}063560_{—}6, TGME49_{—}064740_{—}4, TGME49_{—}066760_{—}1, TGME49_{—}067350_{—}1, TGME49_{—}068590_{—}4, TGME49_{—}068590_{—}9, TGME49_{—}070220_{—}3, TGME49_{—}070250_{—}1, TGME49_{—}070250_{—}2, TGME49_{—}072290_{—}1, TGME49_{—}073380_{—}3, TGME49_{—}074060_{—}5, TGME49_{—}074190_{—}2, TGME49_{—}078660_{—}9, TGME49_{—}085240_{—}1, TGME49_{—}085240_{—}3, TGME49_{—}086120_{—}1, TGME49_{—}086450_{—}1, TGME49_{—}088400_{—}9, TGME49_{—}088500_{—}5, TGME49_{—}089380_{—}3, TGME49_{—}089730_{—}4, TGME49_{—}090580_{—}5, TGME49_{—}090870_{—}5, TGME49_{—}090950_{—}5, TGME49_{—}092220_{—}1, TGME49_{—}095650_{—}4, TGME49_{—}097240_{—}6, TGME49_{—}099060_{—}4, TGME49_{—}099060_{—}6, TGME49_{—}100060_{—}2, TGME49_{—}100310_{—}7, TGME49_{—}101270_{—}10, TGME49_{—}105020_{—}9, TGME49_{—}105270_{—}2, TGME49_{—}105510_{—}3, TGME49_{—}105510_{—}5, TGME49_{—}109910_{—}2, TGME49_{—}112600_{—}3, TGME49_{—}113020_{—}8, TGME49_{—}114850_{—}4, TGME49_{—}118460_{—}3, TGME49_{—}16180_{—}1, TGME49_PP2C-hn, TGME49_TLN_{—}1, or fragments thereof. These provide a new and useful tool that can accurately survey T. gondii-induced diseases, providing improved diagnosis of T. gondii related infection(s), and further provide clear, distinct, antigen targets for serodiagnostic, biomarker, vaccine, and therapeutic product development against T. gondii and the diseases and disorders triggered by T. gondii in mammals, birds, and humans.

The invention can be used to identify biologically relevant antigens, sets of antigens, antibodies, and sets of antibodies from T. gondii and T. gondii-related infections and diseases. The invention can also enable the monitoring and analysis of treatment efficacy, via longitudinal monitoring of reactivity of an antibody, or a set of antibodies, against select T. gondii antigens or sets of antigens. The invention also provides for the detection of antibody reactivity to specific T. gondii protein antigens, or antigen sets, which are important in the diagnosis and treatment of T. gondii-triggered diseases such as toxoplasmosis. Contemplated embodiments include but are not limited to compositions, devices, and methods comprising antibody reactive antigens from T. gondii that can be used as a vaccine, as diagnostic markers, and as therapeutic agents. In preferred embodiments, the T. gondii antigens have quantified and known relative reactivities with respect to sera of a population infected with T. gondii, and have a known association with a disease parameter.

Thus, the invention provides for the identification, analysis, and monitoring of antibodies to specific T. gondii antigens, or antigen sets, which are important in the diagnosis and/or treatment of various T. gondii-triggered diseases. The invention also provides tools and methods to accurately survey T. gondii infection and diseases via the combination of antibody detection and monitoring and characterized sera samples, especially as they relate to their use in diagnostic and therapeutic compositions and methods.

Various objects, features, aspects and advantages of the present invention will become more apparent from the following detailed description of preferred embodiments of the invention.

### BRIEF DESCRIPTION OF THE DRAWING

FIG. 1 depicts frequency analysis of T. gondii exons. FIG. 1A shows the relationship between numbers of exons per gene (x-axis) verses gene frequency. All genes are grouped by exon frequency per gene from 1 to 63. FIG. 1B shows the relationship between numbers of exons (x-axis) and length of coding sequence (base pairs). All genes are grouped by exon frequency per genes, from 1 to 63.

FIG. 2 depicts heat maps of results from Toxoplasma gondii microarrays probed for IgG from negative individuals (no infection), individuals with acute T. gondii infections, individuals with chronic T. gondii infections with persistent elevated IgM titers, and individuals with typical chronic T. gondii infections. Results of microwell plate assays for T. gondii antigens are also shown. Results are ranked by the average response of the chronic group, and donors sorted from left to right within each of the four patient populations by increasing average signal.

FIG. 3 depicts average signal intensity of significant target antigens probed using anti-human IgG. Mean IgG signal intensities of positive antigens are shown. FIG. 3A shows the p values for negative serum vs. acute T. gondii infection serum. FIG. 3B shows the p values for negative serum vs. chronic T. gondii infection serum with persistent IgM. FIG. 3C shows p values for negative serum vs. chronic T. gondii infection serum. Antigens that are significant in all three comparisons are labeled with “*”, antigens significant in two of the comparisons are labeled with “#”.

FIG. 4 depicts candidate IgG serodiagnostic antigens defined by the microarray. FIG. 4A shows a comparison of IgG signal intensity from sera obtained from negative and infected populations for eighteen T. gondii antigens. Average signal intensities for negative, acutely infected, chronically infected with persistent IgM, and chronically infected populations are shown for these eighteen antigens, ranked by descending response of the IgM persisting population. FIG. 4B shows a comparison of IgG signal intensity from sera obtained from negative and infected populations for nine T. gondii antigens. Shown in the histogram are the average signal intensities for each of the four patient populations for these nine antigens, ranked by descending response of the chronic population. FIG. 1C shows dot plots of responses of individual donors to the 086540_{—}1, PP2C-hn, and 070250_{—}2 antigens. Horizontal bars in each population represent the mean. N=negative; A=acute; C/M=chronic/IgM persisting; C=chronic.

FIG. 5 depicts heat maps of results from Toxoplasma gondii microarrays probed for IgM from negative individuals (no infection), individuals with acute T. gondii infections, individuals with chronic T. gondii infections with persistent elevated IgM titers, and individuals with typical chronic T. gondii infections. Results of microwell plate assays for T. gondii antigens are also shown. Antigens are ranked by the average response of the acute group, and donors sorted from left to right within each of the four patient groups by increasing average signal to all 108 antigens.

FIG. 6 depicts average signal intensity of significant target antigens probed using anti-human IgM. Mean IgM signal intensities of positive antigens are shown. FIG. 6A shows the p values for negative serum vs. acute T. gondii infection serum for 40 select antigens. FIG. 6B shows the p values for negative serum vs. chronic T. gondii infection serum with persistent IgM for 30 select antigens. FIG. 6C shows p values for negative serum vs. chronic T. gondii infection serum for 3 select antigens. Antigens that were significant in both negative vs. acute and negative vs. IgM persisting group were labeled with “*”.

FIG. 7 depicts candidate IgM serodiagnostic antigens defined by the microarray. IgM profiles of acute and chronic/IgM persisting populations were compared using T-tests. FIG. 7A shows ninety-one antigens identified by comparison between acute and chronic populations. Shown in the histogram are the average signal intensities for each of the four patient populations for these antigens, ranked by descending response of the acute population. P-values are overlaid. FIG. 7B shows eight antigens identified by comparison between acute and chronic with persistent IgM populations. Shown in the histogram are the average signal intensities for each of the four patient populations for these eight antigens, ranked by descending response of the acute population. P-values are overlaid. FIG. 7C shows dot plots of responses of individual donors to the 046330_{—}5, 044280_{—}1, 023540_{—}10, 048670_{—}5 and 088400_{—}9 antigens. Horizontal bars in each population represent the mean. N=negative; A=acute; C/M=chronic/IgM persisting; C=chronic.

FIG. 8 depicts Area Under Curve (AUC) box plots and Receiver Operator Characteristic (ROC) curves for multiple antigens. FIG. 8A shows a cross validation AUC boxplot for potential IgG serodiagnostic antigens. FIG. 8B illustrates ROC results showing classifiers for potential IgG serodiagnostic antigens. FIG. 8C shows a cross validation AUC boxplot for potential IgM serodiagnostic antigens. 8D illustrates ROC results showing classifiers for potential IgM serodiagnostic antigens.

FIG. 9 depicts overlap between IgM and IgG profiles. FIG. 9A shows scatter plots of corresponding mean IgG and IgM responses against 126 IgM and IgG target antigens that discriminate between seronegative and seropositive individuals. Each panel shows the data for each stage of infection. FIG. 9B shows side by side heat maps from T. gondii antigen arrays for both IgG and IgM responses from uninfected controls and individuals with acute infections, chronic infections with persisting IgM, and chronic infections.

### DETAILED DESCRIPTION

The inventors have discovered various antigens from Toxoplasma gondii that are suitable for diagnostic and therapeutic purposes. Particularly preferred immunodominant antigens and are those encoded by nucleic acids having a sequence according to SEQ ID NO: 1 to SEQ ID NO: 100, and it is generally contemplated that such antigens can be used as single antigens, or in combination (optionally also in combination with antigens from another pathogen) in the manufacture of various diagnostic devices, therapeutic compositions, and vaccines. Preferably, the immunodominant antigens suitable for diagnostic and therapeutic purposes are encoded by the sequences designated TGME49_{—}000470_{—}1 (SEQ ID NO: 1), TGME49_{—}001390_{—}1 (SEQ ID NO:2), TGME49_{—}004130_{—}13 (SEQ ID NO:3), TGME49_{—}005300_{—}6 (SEQ ID NO:4), TGME49_{—}005360_{—}14 (SEQ ID NO:5), TGME49_{—}005740_{—}9 (SEQ ID NO:6), TGME49_{—}012300_{—}5 (SEQ ID NO:7), TGME49_{—}013340_{—}4 (SEQ ID NO:8), TGME49_{—}014610_{—}11 (SEQ ID NO:9), TGME49_{—}014760_{—}2 (SEQ ID NO:10), TGME49_{—}016180_{—}1 (SEQ ID NO: 11), TGME49_{—}016380_{—}13 (SEQ ID NO:12), TGME49_{—}016380_{—}5 (SEQ ID NO:13), TGME49_{—}021310_{—}9 (SEQ ID NO:14), TGME49_{—}023540_{—}10 (SEQ ID NO: 15), TGME49_{—}023540_{—}5 (SEQ ID NO:16), TGME49_{—}024190_{—}10 (SEQ ID NO:17), TGME49_{—}024920_{—}4 (SEQ ID NO:18), TGME49_{—}025320_{—}5 (SEQ ID NO: 19), TGME49_{—}026020_{—}8 (SEQ ID NO:20), TGME49_{—}026110_{—}4 (SEQ ID NO:21), TGME49_{—}026730_{—}9 (SEQ ID NO:22), TGME49_{—}027620_{—}2 (SEQ ID NO:23), TGME49_{—}031430_{—}2 (SEQ ID NO:24), TGME49_{—}033710_{—}4 (SEQ ID NO:25), TGME49_{—}034410_{—}17 (SEQ ID NO:26), TGME49_{—}035020_{—}9, (SEQ ID NO:27) TGME49_{—}035160_{—}2 (SEQ ID NO:28), TGME49_{—}035660_{—}1 (SEQ ID NO:29), TGME49_{—}037150_{—}5 (SEQ ID NO:30), TGME49_{—}040870_{—}16 (SEQ ID NO:31), TGME49_{—}042790_{—}18 (SEQ ID NO:32), TGME49_{—}043580_{—}1 (SEQ ID NO:33), TGME49_{—}044040_{—}8 (SEQ ID NO:34), TGME49_{—}044080_{—}3 (SEQ ID NO:35), TGME49_{—}044280_{—}1 (SEQ ID NO:36), TGME49_{—}045500_{—}3 (SEQ ID NO:37), TGME49_{—}046330_{—}5 (SEQ ID NO:38), TGME49_{—}046340_{—}2 (SEQ ID NO:39), TGME49_{—}047370_{—}4 (SEQ ID NO:40), TGME49_{—}047370_{—}9 (SEQ ID NO:41), TGME49_{—}048200_{—}3 (SEQ ID NO:42), TGME49_{—}048670_{—}5 (SEQ ID NO:43), TGME49_{—}048840_{—}2 (SEQ ID NO:44), TGME49_{—}048840_{—}3 (SEQ ID NO:45), TGME49_{—}054370_{—}11 (SEQ ID NO:46), TGME49_{—}054570_{—}6 (SEQ ID NO:47), TGME49_{—}057080_{—}5 (SEQ ID NO:48), TGME49_{—}057080_{—}5 (SEQ ID NO:49), TGME49_{—}057520_{—}1 (SEQ ID NO:50), TGME49_{—}058390_{—}1 (SEQ ID NO:51), TGME49_{—}058980_{—}1 (SEQ ID NO:52), TGME49_{—}059200_{—}2 (SEQ ID NO:53), TGME49_{—}061740_{—}1 (SEQ ID NO:54), TGME49_{—}062920_{—}5 (SEQ ID NO:55), TGME49_{—}063560_{—}6 (SEQ ID NO:56), TGME49_{—}064740_{—}4 (SEQ ID NO:57), TGME49_{—}066760_{—}1 (SEQ ID NO:58), TGME49_{—}067350_{—}1 (SEQ ID NO:59), TGME49_{—}068590_{—}4 (SEQ ID NO:60), TGME49_{—}068590_{—}9 (SEQ ID NO:61), TGME49_{—}070220_{—}3 (SEQ ID NO:62), TGME49_{—}070250_{—}2 (SEQ ID NO:63), TGME49_{—}072290_{—}1 (SEQ ID NO:64), TGME49_{—}073380_{—}3 (SEQ ID NO:65), TGME49_{—}073380_{—}3 (SEQ ID NO:66), TGME49_{—}074060_{—}5 (SEQ ID NO:67), TGME49_{—}074190_{—}2 (SEQ ID NO:68), TGME49_{—}078660_{—}9 (SEQ ID NO:69), TGME49_{—}085240_{—}1 (SEQ ID NO:70), TGME49_{—}085240_{—}3 (SEQ ID NO:71), TGME49_{—}086120_{—}1 (SEQ ID NO:72), TGME49_{—}086450_{—}1 (SEQ ID NO:73), TGME49_{—}088400_{—}9 (SEQ ID NO:74), TGME49_{—}088500_{—}5 (SEQ ID NO:75), TGME49_{—}089380_{—}3 (SEQ ID NO:76), TGME49_{—}089730_{—}4 (SEQ ID NO:77), TGME49_{—}090580_{—}5 (SEQ ID NO:78), TGME49_{—}090870_{—}5 (SEQ ID NO:79), TGME49_{—}090950_{—}5 (SEQ ID NO:80), TGME49_{—}092220_{—}1 (SEQ ID NO:81), TGME49_{—}095650_{—}4 (SEQ ID NO:82), TGME49_{—}097240_{—}6 (SEQ ID NO:83), TGME49_{—}099060_{—}4 (SEQ ID NO:84), TGME49_{—}099060_{—}6 (SEQ ID NO:85), TGME49_{—}100060_{—}2 (SEQ ID NO:86), TGME49_{—}100310_{—}7 (SEQ ID NO:87), TGME49_{—}101270_{—}10 (SEQ ID NO:88), TGME49_{—}105020_{—}9 (SEQ ID NO:89), TGME49_{—}105270_{—}2 (SEQ ID NO:90), TGME49_{—}105510_{—}3 (SEQ ID NO:91), TGME49_{—}105510_{—}5 (SEQ ID NO:92), TGME49_{—}109910_{—}2 (SEQ ID NO:93), TGME49_{—}112600_{—}3 (SEQ ID NO:94), TGME49_{—}113020_{—}8 (SEQ ID NO:95), TGME49_{—}114850_{—}4 (SEQ ID NO:96), TGME49_{—}118460_{—}3 (SEQ ID NO:97), TGME49_{—}16180_{—}1 (SEQ ID NO:98), TGME49_PP2C-hn (SEQ ID NO:99), TGME49_TLN_{—}1 (SEQ ID NO: 100), and fragments thereof.

As used herein, the term “immunodominant antigen” refers to an antigen that elicits in at least one stage of the infection production of one or more types of antibodies (e.g., IgG, IgA, IgE, IgM, etc.) in at least 20%, more typically at least 40%, and most typically at least 70% of a population exposed to the antigen, or wherein, when compared to other antigens of the same pathogen, the average binding affinity and/or average quantity of the antibodies produced in the patient in at least one stage of the disease is at least in the upper half, more typically upper tertile, and most typically upper quartile.

In a preferred embodiment, IgG and IgM from infected and non-infected individuals may be used to identify immunodominant antigens characteristic of a disease state. In other embodiments IgA, IgE, IgD, and IgY can be used for characterization of immunodominant antigens. In still other embodiments antibodies from different classes can be used in combination to identify immunodominant antigens. Most typically, the average binding affinity and/or average quantity of the antibodies is reflected in a signal intensity associated with the corresponding antigen in an assay, and signal intensity can therefore be used as a surrogate marker for average binding affinity and/or average quantity of the antibodies. In further aspects, preferred immunodominant antigens are also characterized by a response in the test group that is considered statistically significant when compared with control signal intensity derived from an uninfected negative control group, wherein the significance level p is preferably equal or less than 0.1, more preferably equal or less than 0.05, and most preferably equal or less than 0.01.

In one aspect of the inventive subject matter, immunodominant antigens are identified from a proteome screen against sera of a population that has been previously exposed to the pathogen. Most preferably, the population is subdivided in several sub-populations to reflect various disease parameters (e.g., acute disease, chronic disease, chronic disease with persistent IgM, time since infection, gestational status, presence of co-infection with HIV, absence of infection, etc.), which can then be correlated with antibody responses to the so identified antigens. It is still further preferred that the screening also provides data on relative reactivities with respect to the antigens and sera of the populations/sub-populations.

It is generally preferred that at least part of the pathogen's genome is obtained and all potential open reading frames and portions thereof are determined in silico. Once the potential genes are identified, suitable primers are determined to provide amplicons of the entire Open Reading Frames (ORFs), or, less preferably, portions thereof, wherein the primers are preferably designed to allow facile subcloning into an expression system. Most preferably, the subcloning uses recombinase-based subcloning using unpurified PCR mixtures to avoid cloning bias, and the so obtained recombinant plasmids are polyclonally multiplied, which enables unbiased presentation of the amplicons. It is still further particularly preferred that the plasmid preparations are then subjected to an in vitro transcription/translation reaction to thereby provide the recombinant ORF peptide, which is then spotted or otherwise immobilized onto a suitable addressable carrier (e.g., membrane, bead, etc.).

It should be recognized that the so prepared proteomes can then be exposed to serum of a population of control individuals and/or population of individuals that are known to have current or previous exposure to the above pathogen from which the ORFs were prepared. Antibodies present in these sera and that bind to one or more of the ORFs are then detected using well known methods (e.g., use of secondary antibodies). These methods may permit further resolution of the antibody population into immunoglobulin classes, including IgG, IgM, IgA, IgE, and IgD, the distribution of which can permit further clinical insight. In this manner, the entire proteome of the pathogen can be rapidly assessed for immunogenicity and potential binding with antibodies in serum. Various preferred aspects, compositions, and methods of proteome preparation are disclosed in International patent publication number WO 06/088492, which is incorporated by reference herein in its entirety.

Therefore, and among various other advantages, it should be especially recognized that contemplated compositions and methods presented herein will allow for preparation of vaccines and diagnostic compositions comprising one or more antigens with known and predetermined affinity to target ORFs of a pathogen. As individual immune systems are known to exhibit significant variation with respect to antigen recognition, methods and compositions contemplated herein will allow statistically supported antigen identification to identify one or more immunodominant antigens in a population of patients. Consequently, multiple targets can be used to elicit an immune response and/or detect a prior exposure, even where one or more of the targets may be evasive for detection or elicit only a weak immune response.

With respect to the immunodominant sequences identified herein, it should be further appreciated that the sequences need not be complete ORFs, but that suitable sequences may also be partial sequences (e.g., synthetic, recombinant or isolated) that typically comprise at least a portion of an antigenic epitope. In addition, contemplated nucleic acid sequences include those that will hybridize under stringent hybridization conditions to the respective sequences listed in the sequence listing. Thus, sequences contemplated herein may be identified as DNA sequences encoding the antigenic peptide (either whole or in part), or may be identified as a peptide sequence (or homologs thereof). Similarly, chemically modified antigens, and/or orthologs of the polypeptides presented herein are also deemed suitable for use herein.

It should be particularly noted that while proteome screening can provide a plurality of antigens suitable for use in diagnosis, vaccination, and/or therapy, such an approach only provides an approximation of the individual responses. Therefore, as most individual immune reactions towards the same pathogen elicit a significantly distinct profile of antibodies (e.g., depending on disease stage, previous exposure, and/or inter-individual variability), results obtained from such screening are typically inhomogeneous. Consequently, the inherent variability of the individual immune responses and variability of the quantity of recombinant protein immobilized on the array must be taken into consideration in order to obtain meaningful results.

Therefore, it should be appreciated that filtering of raw data will result in a collection of antigens with quantified and known relative reactivities with respect to sera of a population infected with the pathogen. Moreover, it should be noted that as results may be specific to a particular stage in the course of an infection, relative reactivities may be indicative of the time course of the infection, and/or relative reactivities may represent differences in the strength of immunogenicity of the particular antigen or quantity of deposited antigen in the screening assay. Additionally, it should be particularly recognized that depending on the choice of the specific patient population, the tested sera will reflect the immune status of a population that is characterized by one or more parameters of the disease. For example, populations may be observed that are infected or not infected, that have acute infections, that have had a long-term exposure or chronic infection, that have coexisting infections, that are pregnant, that represent a group of responders (or non-responders) to a particular drug treatment, or that have at least partial immunity to the pathogen.

In still further contemplated aspects, immunodominant antigens are identified by selecting for an antigen (preferably within a well-defined sub-population) that (a) produces in at least 40-50% of a population a measurable signal, and (b) has a signal strength of at least 40% of the overall average signal intensity. However, and more preferably, the signal strength will be at least above average of the overall average signal intensity, and even more preferably in the upper tertile (quartile, or even quintile) of signal intensities in the assay. Therefore, and viewed from another perspective, immunodominant antigens will preferably be selected in a comparison of at least two series of tests, wherein one series of tests is typically the sub-population (e.g., primary infection, active disease, latent infection, recovering, previously diseased, chronic, etc.) and the other series of tests is the control group (e.g., other sub-population or control group). Still further, it is generally preferred that the series of tests also include a negative control against which the potential immunodominant antigens are compared.

Consequently, and with particular respect to the pathogen presented herein, it should be appreciated that compositions comprising one or more selected immunodominant antigens can be prepared that will have a statistically high probability to elicit or have elicited an immune response in a relatively large group of patients. Further, where the antigens are determined from selected sub-populations (e.g., acute infection, chronic infection, coexisting infection, pregnancy, etc.), the antigens also have a known association with a disease parameter and thus allow staging of the disease and/or prediction of therapeutic efficacy. Moreover, as the antigens presented herein are immunodominant antigens, it should be noted that vaccine compositions can be prepared with known or predictable immunogenicity.

More specifically, antigens from Toxoplasma gondii encoded by the nucleic acids of SEQ ID NO: 1 to SEQ ID NO: 100 were identified as immunodominant (see examples below). With respect to the reading frame for each of the sequences of SEQ ID NO: 1 to SEQ ID NO: 100, it should be noted that the first base in the sequences is either the first base of the start codon or the first base in the first codon of the polypeptide that was identified with the methods and compositions provided herein. Most typically, the last three bases denote the stop codon, or the last base of the last codon of the polypeptide that was identified with the methods and compositions provided herein.

In these examples, each of the antigens was characterized, inter alia, with regard to their individual and relative reactivities for the pathogen. Most typically, reactivity was measured as strength of immunogenicity (e.g., such that average binding affinity and/or average quantity of the antibodies produced a predetermined signal intensity (e.g., in the upper half, upper tertile, or even upper quartile)). Viewed from a different perspective, each one of the identified antigens has a known signal strength (reflecting the quantity of antibodies formed in the patient) in the assay as described below relative to another one of the identified antigens. Furthermore, each of the identified antigens was also characterized by association with at least one parameter. In most cases, the disease parameter was acute infection, chronic infection, and chronic infection with persistent IgM. Therefore, it should be especially appreciated that identification of immunodominant antigens will not only allow for identification of statistically meaningful antigens for diagnosis, vaccine development, and treatment, but also allow to develop a stage specific tool to identify candidate molecules to fine-tune diagnosis and/or treatment.

Therefore, in one embodiment, the invention concerns a method of predicting the likelihood of a host being infected by T. gondii, comprising determining IgG reactivity against one or more antigens, or their variants, in a serum or other body fluid sample obtained from a host, wherein the antigen is selected from the group consisting of TGME49_{—}001390_{—}1, TGME49_{—}005300_{—}6, TGME49_{—}016380_{—}13, TGME49_{—}024190_{—}10, TGME49_{—}026020_{—}8, TGME49_{—}031430_{—}2, TGME49_{—}033710_{—}4, TGME49_{—}035020_{—}9, TGME49_{—}037150_{—}5, TGME49_{—}048840_{—}2, TGME49_{—}054370_{—}11, TGME49_{—}057080_{—}5, TGME49_{—}062920_{—}5, TGME49_{—}064740_{—}4, TGME49_{—}066760_{—}1, TGME49_{—}067350_{—}1, TGME49_{—}068590_{—}9, TGME49_{—}070220_{—}3, TGME49_{—}085240_{—}3, TGME49_{—}086450_{—}1, TGME49_{—}089730_{—}4, TGME49_{—}090580_{—}5, TGME49_{—}090870_{—}5, TGME49_{—}090950_{—}5, TGME49_{—}099060_{—}4, TGME49_{—}100310_{—}7, TGME49_{—}105510_{—}3, TGME49_{—}113020_{—}8, TGME49_PP2C-hn, TGME49_TLN-1, and fragments thereof; wherein antibody reactivity against one or more of TGME49_{—}001390_{—}1, TGME49_{—}005300_{—}6, TGME49_{—}016380_{—}13, TGME49_{—}024190_{—}10, TGME49_{—}026020_{—}8, TGME49_{—}031430_{—}2, TGME49_{—}033710_{—}4, TGME49_{—}035020_{—}9, TGME49_{—}037150_{—}5, TGME49_{—}048840_{—}2, TGME49_{—}054370_{—}11, TGME49_{—}057080_{—}5, TGME49_{—}062920_{—}5, TGME49_{—}064740_{—}4, TGME49_{—}066760_{—}1, TGME49_{—}067350_{—}1, TGME49_{—}068590_{—}9, TGME49_{—}070220_{—}3, TGME49_{—}085240_{—}3, TGME49_{—}086450_{—}1, TGME49_{—}089730_{—}4, TGME49_{—}090580_{—}5, TGME49_{—}090870_{—}5, TGME49_{—}090950_{—}5, TGME49_{—}099060_{—}4, TGME49_{—}100310_{—}7, TGME49_{—}105510_{—}3, TGME49_{—}113020_{—}8, TGME49_PP2C-hn, TGME49_TLN-1, and fragments thereof indicates an increased likelihood of the host being infected by T. gondii.

In another embodiment, the invention concerns a method of predicting the likelihood of a host being infected by T. gondii, comprising determining IgM reactivity against one or more antigens, or their variants, in a serum or other body fluid sample obtained from a host, wherein the antigen is selected from the group consisting of TGME49_{—}004130_{—}13, TGME49_{—}005360_{—}14, TGME49_{—}012300_{—}5, TGME49_{—}014610_{—}11, TGME49_{—}014760_{—}2, TGME49_{—}016380_{—}13, TGME49_{—}023540_{—}5, TGME49_{—}024190_{—}10, TGME49_{—}024920_{—}4, TGME49_{—}025320_{—}5, TGME49_{—}026730_{—}9, TGME49_{—}031430_{—}2, TGME49_{—}033710_{—}4, TGME49_{—}034410_{—}17, TGME49_{—}035160_{—}2, TGME49_{—}037150_{—}5, TGME49_{—}040870_{—}16, TGME49_{—}042790_{—}18, TGME49_{—}043580_{—}1, TGME49_{—}044080_{—}3, TGME49_{—}044280_{—}1, TGME49_{—}045500_{—}3, TGME49_{—}046330_{—}5, TGME49_{—}047370_{—}9, TGME49_{—}048200_{—}3, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057520_{—}1, TGME49_{—}058980_{—}1, TGME49_{—}059200_{—}2, TGME49_{—}061740_{—}1, TGME49_{—}064740_{—}4, TGME49_{—}073380_{—}3, TGME49_{—}074190_{—}2, TGME49_{—}078660_{—}9, TGME49_{—}085240_{—}1, TGME49_{—}086120_{—}1, TGME49_{—}088500_{—}5, TGME49_{—}089380_{—}3, TGME49_{—}092220_{—}1, TGME49_{—}095650_{—}4, TGME49_{—}097240_{—}6, TGME49_{—}099060_{—}4, TGME49_{—}100060_{—}2, TGME49_{—}100310_{—}7, TGME49_{—}101270_{—}10, TGME49_{—}105270_{—}2, TGME49_{—}105510_{—}5, TGME49_{—}112600_{—}3, TGME49_{—}114850_{—}4, TGME49_{—}16180_{—}1 TGME49_TLN_{—}1, and fragments thereof; wherein antibody reactivity against one or more of TGME49_{—}004130_{—}13, TGME49_{—}005360_{—}14, TGME49_{—}012300_{—}5, TGME49_{—}014610_{—}11, TGME49_{—}014760_{—}2, TGME49_{—}016380_{—}13, TGME49_{—}023540_{—}5, TGME49_{—}024190_{—}10, TGME49_{—}024920_{—}4, TGME49_{—}025320_{—}5, TGME49_{—}026730_{—}9, TGME49_{—}031430_{—}2, TGME49_{—}033710_{—}4, TGME49_{—}034410_{—}17, TGME49_{—}035160_{—}2, TGME49_{—}037150_{—}5, TGME49_{—}040870_{—}16, TGME49_{—}042790_{—}18, TGME49_{—}043580_{—}1, TGME49_{—}044080_{—}3, TGME49_{—}044280_{—}1, TGME49_{—}045500_{—}3, TGME49_{—}046330_{—}5, TGME49_{—}047370_{—}9, TGME49_{—}048200_{—}3, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057520_{—}1, TGME49_{—}058980_{—}1, TGME49_{—}059200_{—}2, TGME49_{—}061740_{—}1, TGME49_{—}064740_{—}4, TGME49_{—}073380_{—}3, TGME49_{—}074190_{—}2, TGME49_{—}078660_{—}9, TGME49_{—}085240_{—}1, TGME49_{—}086120_{—}1, TGME49_{—}088500_{—}5, TGME49_{—}089380_{—}3, TGME49_{—}092220_{—}1, TGME49_{—}095650_{—}4, TGME49_{—}097240_{—}6, TGME49_{—}099060_{—}4, TGME49_{—}100060_{—}2, TGME49_{—}100310_{—}7, TGME49_{—}101270_{—}10, TGME49_{—}105270_{—}2, TGME49_{—}105510_{—}5, TGME49_{—}112600_{—}3, TGME49_{—}114850_{—}4, TGME49_{—}16180_{—}1 TGME49_TLN_{—}1, and fragments thereof indicates an increased likelihood of the host being infected by T. gondii.

In another embodiment, the invention concerns a method of predicting the likelihood of a host having an acute infection with T. gondii, comprising determining IgG reactivity against one or more antigens, or their variants, in a serum or other body fluid sample obtained from a host, wherein the antigen is selected from the group consisting of TGME49_{—}000470_{—}1, TGME49_{—}013340_{—}4, TGME49_{—}021310_{—}9, TGME49_{—}024190_{—}10, TGME49_{—}026020_{—}8, TGME49_{—}026110_{—}4, TGME49_{—}027620_{—}2, TGME49_{—}035020_{—}9, TGME49_{—}046340_{—}2, TGME49_{—}047370_{—}4, TGME49_{—}054370_{—}11, TGME49_{—}057080_{—}5, TGME49_{—}058390_{—}1, TGME49_{—}062920_{—}5, TGME49_{—}066760_{—}1, TGME49_{—}068590_{—}9, TGME49_{—}070220_{—}3, TGME49_{—}070250_{—}2, TGME49_{—}086450_{—}1, TGME49_{—}089730_{—}4, TGME49_{—}090950_{—}5, TGME49_{—}099060_{—}4, TGME49_{—}105510_{—}3, TGME49_{—}105510_{—}5, TGME49_PP2C-hn, and fragments thereof; wherein antibody reactivity against one or more of TGME49_{—}000470_{—}1, TGME49_{—}013340_{—}4, TGME49_{—}021310_{—}9, TGME49_{—}024190_{—}10, TGME49_{—}026020_{—}8, TGME49_{—}026110_{—}4, TGME49_{—}027620_{—}2, TGME49_{—}035020_{—}9, TGME49_{—}046340_{—}2, TGME49_{—}047370_{—}4, TGME49_{—}054370_{—}11, TGME49_{—}057080_{—}5, TGME49_{—}058390_{—}1, TGME49_{—}062920_{—}5, TGME49_{—}066760_{—}1, TGME49_{—}068590_{—}9, TGME49_{—}070220_{—}3, TGME49_{—}070250_{—}2, TGME49_{—}086450_{—}1, TGME49_{—}089730_{—}4, TGME49_{—}090950_{—}5, TGME49_{—}099060_{—}4, TGME49_{—}105510_{—}3, TGME49_{—}105510_{—}5, TGME49_PP2C-hn, and fragments thereof indicates an increased likelihood of the host having an acute infection with T. gondii.

In yet another embodiment, the invention concerns a method of predicting the likelihood of a host having an acute infection with T. gondii, comprising determining IgM reactivity against one or more antigens, or their variants, in a serum or other body fluid sample obtained from a host, wherein the antigen is selected from the group consisting of TGME49_{—}005740_{—}9 TGME49_{—}016180_{—}1 TGME49_{—}016380_{—}5 TGME49_{—}023540_{—}10 TGME49_{—}026730_{—}9 TGME49_{—}042790_{—}18 TGME49_{—}044040_{—}8 TGME49_{—}044280_{—}1 TGME49_{—}045500_{—}3 TGME49_{—}046330_{—}5 TGME49_{—}048670_{—}5 TGME49_{—}063560_{—}6 TGME49_{—}064740_{—}4 TGME49_{—}068590_{—}4 TGME49_{—}072290_{—}1 TGME49_{—}074060_{—}5 TGME49_{—}078660_{—}9 TGME49_{—}088400_{—}9 TGME49_{—}095650_{—}4 TGME49_{—}099060_{—}6 TGME49_{—}105020_{—}9 TGME49_{—}109910_{—}2 TGME49_{—}114850_{—}4 TGME49_{—}118460_{—}3 TGME49_TLN-1, and fragments thereof; wherein antibody reactivity against one or more of TGME49_{—}005740_{—}9 TGME49_{—}016180_{—}1 TGME49_{—}016380_{—}5 TGME49_{—}023540_{—}10 TGME49_{—}026730_{—}9 TGME49_{—}042790_{—}18 TGME49_{—}044040_{—}8 TGME49_{—}044280_{—}1 TGME49_{—}045500_{—}3 TGME49_{—}046330_{—}5 TGME49_{—}048670_{—}5 TGME49_{—}063560_{—}6 TGME49_{—}064740_{—}4 TGME49_{—}068590_{—}4 TGME49_{—}072290_{—}1 TGME49_{—}074060_{—}5 TGME49_{—}078660_{—}9 TGME49_{—}088400_{—}9 TGME49_{—}095650_{—}4 TGME49_{—}099060_{—}6 TGME49_{—}105020_{—}9 TGME49_{—}109910_{—}2 TGME49_{—}114850_{—}4 TGME49_{—}118460_{—}3 TGME49_TLN-1, and fragments thereof indicates an increased likelihood of the host having an acute infection with T. gondii.

For example, suitable diagnostic devices especially include those comprising one or more of the immunodominant antigens, fragments, or analogs thereof that are encoded by nucleic acids according to SEQ ID NO:1 to SEQ ID NO: 100, preferably TGME49_{—}000470_{—}1 (SEQ ID NO:1), TGME49_{—}001390_{—}1 (SEQ ID NO:2), TGME49_{—}004130_{—}13 (SEQ ID NO:3), TGME49_{—}005300_{—}6 (SEQ ID NO:4), TGME49_{—}005360_{—}14 (SEQ ID NO:5), TGME49_{—}005740_{—}9 (SEQ ID NO:6), TGME49_{—}012300_{—}5 (SEQ ID NO:7), TGME49_{—}013340_{—}4 (SEQ ID NO:8), TGME49_{—}014610_{—}11 (SEQ ID NO:9), TGME49_{—}014760_{—}2 (SEQ ID NO:10), TGME49_{—}016180_{—}1 (SEQ ID NO:11), TGME49_{—}016380_{—}13 (SEQ ID NO:12), TGME49_{—}016380_{—}5 (SEQ ID NO:13), TGME49_{—}021310_{—}9 (SEQ ID NO:14), TGME49_{—}023540_{—}10 (SEQ ID NO:15), TGME49_{—}023540_{—}5 (SEQ ID NO:16), TGME49_{—}024190_{—}10 (SEQ ID NO:17), TGME49_{—}024920_{—}4 (SEQ ID NO:18), TGME49_{—}025320_{—}5 (SEQ ID NO:19), TGME49_{—}026020_{—}8 (SEQ ID NO:20), TGME49_{—}026110_{—}4 (SEQ ID NO:21), TGME49_{—}026730_{—}9 (SEQ ID NO:22), TGME49_{—}027620_{—}2 (SEQ ID NO:23), TGME49_{—}031430_{—}2 (SEQ ID NO:24), TGME49_{—}033710_{—}4 (SEQ ID NO:25), TGME49_{—}034410_{—}17 (SEQ ID NO:26), TGME49_{—}035020_{—}9, (SEQ ID NO:27) TGME49_{—}035160_{—}2 (SEQ ID NO:28), TGME49_{—}035660_{—}1 (SEQ ID NO:29), TGME49_{—}037150_{—}5 (SEQ ID NO:30), TGME49_{—}040870_{—}16 (SEQ ID NO:31), TGME49_{—}042790_{—}18 (SEQ ID NO:32), TGME49_{—}043580_{—}1 (SEQ ID NO:33), TGME49_{—}044040_{—}8 (SEQ ID NO:34), TGME49_{—}044080_{—}3 (SEQ ID NO:35), TGME49_{—}044280_{—}1 (SEQ ID NO:36), TGME49_{—}045500_{—}3 (SEQ ID NO:37), TGME49_{—}046330_{—}5 (SEQ ID NO:38), TGME49_{—}046340_{—}2 (SEQ ID NO:39), TGME49_{—}047370_{—}4 (SEQ ID NO:40), TGME49_{—}047370_{—}9 (SEQ ID NO:41), TGME49_{—}048200_{—}3 (SEQ ID NO:42), TGME49_{—}048670_{—}5 (SEQ ID NO:43), TGME49_{—}048840_{—}2 (SEQ ID NO:44), TGME49_{—}048840_{—}3 (SEQ ID NO:45), TGME49_{—}054370_{—}11 (SEQ ID NO:46), TGME49_{—}054570_{—}6 (SEQ ID NO:47), TGME49_{—}057080_{—}5 (SEQ ID NO:48), TGME49_{—}057080_{—}5 (SEQ ID NO:49), TGME49_{—}057520_{—}1 (SEQ ID NO:50), TGME49_{—}058390_{—}1 (SEQ ID NO:51), TGME49_{—}058980_{—}1 (SEQ ID NO:52), TGME49_{—}059200_{—}2 (SEQ ID NO:53), TGME49_{—}061740_{—}1 (SEQ ID NO:54), TGME49_{—}062920_{—}5 (SEQ ID NO:55), TGME49_{—}063560_{—}6 (SEQ ID NO:56), TGME49_{—}064740_{—}4 (SEQ ID NO:57), TGME49_{—}066760_{—}1 (SEQ ID NO:58), TGME49_{—}067350_{—}1 (SEQ ID NO:59), TGME49_{—}068590_{—}4 (SEQ ID NO:60), TGME49_{—}068590_{—}9 (SEQ ID NO:61), TGME49_{—}070220_{—}3 (SEQ ID NO:62), TGME49_{—}070250_{—}2 (SEQ ID NO:63), TGME49_{—}072290_{—}1 (SEQ ID NO:64), TGME49_{—}073380_{—}3 (SEQ ID NO:65), TGME49_{—}073380_{—}3 (SEQ ID NO:66), TGME49_{—}074060_{—}5 (SEQ ID NO:67), TGME49_{—}074190_{—}2 (SEQ ID NO:68), TGME49_{—}078660_{—}9 (SEQ ID NO:69), TGME49_{—}085240_{—}1 (SEQ ID NO:70), TGME49_{—}085240_{—}3 (SEQ ID NO:71), TGME49_{—}086120_{—}1 (SEQ ID NO:72), TGME49_{—}086450_{—}1 (SEQ ID NO:73), TGME49_{—}088400_{—}9 (SEQ ID NO:74), TGME49_{—}088500_{—}5 (SEQ ID NO:75), TGME49_{—}089380_{—}3 (SEQ ID NO:76), TGME49_{—}089730_{—}4 (SEQ ID NO:77), TGME49_{—}090580_{—}5 (SEQ ID NO:78), TGME49_{—}090870_{—}5 (SEQ ID NO:79), TGME49_{—}090950_{—}5 (SEQ ID NO:80), TGME49_{—}092220_{—}1 (SEQ ID NO:81), TGME49_{—}095650_{—}4 (SEQ ID NO:82), TGME49_{—}097240_{—}6 (SEQ ID NO:83), TGME49_{—}099060_{—}4 (SEQ ID NO:84), TGME49_{—}099060_{—}6 (SEQ ID NO:85), TGME49_{—}100060_{—}2 (SEQ ID NO:86), TGME49_{—}100310_{—}7 (SEQ ID NO:87), TGME49_{—}101270_{—}10 (SEQ ID NO:88), TGME49_{—}105020_{—}9 (SEQ ID NO:89), TGME49_{—}105270_{—}2 (SEQ ID NO:90), TGME49_{—}105510_{—}3 (SEQ ID NO:91), TGME49_{—}105510_{—}5 (SEQ ID NO:92), TGME49_{—}109910_{—}2 (SEQ ID NO:93), TGME49_{—}112600_{—}3 (SEQ ID NO:94), TGME49_{—}113020_{—}8 (SEQ ID NO:95), TGME49_{—}114850_{—}4 (SEQ ID NO:96), TGME49_{—}118460_{—}3 (SEQ ID NO:97), TGME49_{—}16180_{—}1 (SEQ ID NO:98), TGME49_PP2C-hn (SEQ ID NO:99), TGME49_TLN_{—}1 (SEQ ID NO: 100), and fragments thereof.

Depending on the particular device format, the device may have only a single immunodominant antigen, fragment, or analog that may be used for detection of binding of antibodies from blood, plasma or serum or other bodily fluids containing antibody in an automated manner or by visual observation. For example, where a single immunodominant antigen is employed, suitable devices may be in the format of a testing dipstick or competitive ELISA. On the other hand, where multiple immunodominant antigens are employed, suitable devices may be in the format of a testing dipstick with a plurality of test sites or a testing array that can be read in an automated device (e.g., via a scanner) or visual manner (e.g., via a dye-forming colorimetric reaction). Most typically, in such testing arrays the plurality of antigens is deposited in a spatially addressable manner on a planar surface, such as in the wells of a microwell plate or spotted on the surface of a microscope slide. Alternatively such testing arrays may be in the form of a fluid suspension array wherein antigens are coupled to particles held in liquid suspension, where the identity of the coupled antigen is encoded into the particle by particle size, incorporation of a dyes, incorporation of fluors, holographic interference patterns, and so on. Moreover, it should be noted that diagnostic devices contemplated herein may be based on numerous well known manners of detection, including ELISA (sandwich or non-sandwich), competitive ELISA, anti-idiotypic antibodies, etc., wherein all known colorimetric and photometric (e.g., fluorescence, luminescence, turbidimetric, nephelometric, etc.) or radiometric reactions are deemed suitable for use.

In most typical devices, one or more immunodominant antigens of a single (or multiple) pathogen and/or serotype are deposited on a solid surface or onto an addressable solid phase and exposed to blood, serum, plasma or other antibody-containing body fluid. Consequently, so prepared compositions can be employed to identify and/or characterize an immune response of an individual against selected antigens, and optionally assess the kind of immune response (e.g., identification of acute or chronic infection), as well as disease progression, efficacy of therapy, etc. In some embodiments a plurality of antigens is used. A plurality of antigens can include from 2 to 10 antigens, but significantly larger numbers of antigens are also contemplated, including at least 25%, more typically at least 50%, even more typically at least 75%, and most typically at least 90% of the proteome of the pathogen. Similarly, less than 5 antigens (1-4) are also deemed suitable. In some embodiments, the antigens comprise T. gondii antigen variants, including truncated forms, non-glycosylated forms, recombinant forms, chimeric forms, etc. Thus, in some embodiments, the invention comprises two or more of the T. gondii antigens presented hereinabove, immobilized on a surface, wherein the T. gondii antigens may be associated with a single disease or more than one disease.

In still other embodiments, the reactivity level of antibodies to at least 2, or at least 5, or at least 10, or at least 15, or at least 20, or at least 25 antigens is determined. While determination of reactivity can be performed in numerous formats well known in the art, in a preferred embodiment that determination is performed in a multiplex format, for example in an array, ELISA, or testing dipstick format. Thus, arrays, or testing dipsticks having at least one, more typically at least two, even more typically at least 5, or at least 10, or at least 15, or at least 20, or at least 25 antigens are contemplated. In a preferred embodiment ELISA's, or testing dipsticks, having at least one, more typically at least three test antigens are contemplated.

In further typical aspects of the inventive subject matter, contemplated arrays are processed in a microfluidic device. For example, an array of antigens in such devices may be deposited on a membrane or other surface that is then placed in a microfluidic device having either ports or internal reservoirs that permit the introduction of sample and necessary reagents to the array. Depending on the specific configuration, signals may be acquired using optical methods (e.g., CCD chip, flat bed scanner, etc.), electrical methods (e.g., voltametric or amperometric), or other methods well known in the art. Alternatively, visual detection or detection using a conventional flat bed scanner and/or fluorescence detection is also deemed suitable.

As noted above, individual immune responses to Toxoplasma gondii antigens may vary widely. To minimize the impact of the variation one embodiment of the invention concerns a method of predicting the likelihood of a host having a T. gondii disease or disorder, comprising determining prognostic antibody reactivity against one or more specific T. gondii antigens, or their variants, in a serum or other body fluid sample obtained from the host, wherein the antibody reactivity is normalized against the that of a non-prognostic antibody reactivity in the serum sample, or of a reference set of antibody reactivity; wherein antibody reactivity against one or more of said specific T. gondii antigens indicates an increased likelihood of the host having a disease or disorder.

In another embodiment, the invention concerns a method of predicting the likelihood of a host having a T. gondii disease or disorder, comprising determining prognostic antibody reactivity against one or more T. gondii antigens presented hereinabove, or their variants, in a serum or other body fluid sample obtained from the host, normalized against a non-prognostic antibody reactivity in the sample, or of a reference set of autoantibody reactivities; wherein autoantibody reactivity against one or more of the T. gondii antigens presented hereinabove indicates an increased likelihood of the host having a T. gondii-related disease or disorder.

In a further embodiment, the invention can comprise a method of predicting the likelihood of a patient being infected by T. gondii, comprising the steps of (a) determining the reactivity levels of antibodies against T. gondii antigens, or their variants, presented hereinabove in a serum or other body fluid sample obtained from the patient, optionally normalized against the reactivity levels of other antibodies against T. gondii antigens, or their variants, in said sera sample, or of a reference set of autoantibody reactivity levels; (b) subjecting the data obtained in step (a) to statistical analysis; and; (c) determining the likelihood of said patient being infected by T. gondii.

In a still further embodiment, the invention concerns a method of preparing a personalized proteomic and antibody profile for an individual T. gondii patient, comprising the steps of (a) subjecting a sera or other body fluid sample obtained from the patient to protein array analysis; (b) determining the reactivity level of one or more antibodies against T. gondii antigens, or their variants, wherein the reactivity level is optionally normalized against reactivity levels of one or more control antibodies; and (c) creating a report summarizing the data obtained by said analysis. The report may include prediction of the likelihood of severity, or stage, of T. gondii infection in the patient and/or a recommendation for a treatment modality of said patient.

In a further aspect, the inventive subject matter concerns a method for detecting one or more T. gondii antibodies in a patient. The present inventive subject matter also provides tools and methods to accurately survey T. gondii infections via the combination of: antibody detection and monitoring, and characterized sera samples.

In another aspect of the inventive subject matter, T. gondii antigens that triggered antibody reactivities are utilized in an antigen composition that comprises one or more antigens that are characteristic of a T. gondii-induced disease or disorder and are associated with a carrier, wherein the antigens have quantified and known relative reactivities with respect to sera of a population infected with T. gondii, and wherein the antigens have a known association with a T. gondii disease parameter. Most preferably, the antigens are polypeptides (or comprise fragments thereof). In a preferred embodiment, such T. gondii antigens have a sequence according to TGME49_{—}000470_{—}1, TGME49_{—}001390_{—}1, TGME49_{—}004130_{—}13, TGME49_{—}005300_{—}6, TGME49_{—}005360_{—}14, TGME49_{—}005740_{—}9, TGME49_{—}012300_{—}5, TGME49_{—}013340_{—}4, TGME49_{—}014610_{—}11, TGME49_{—}014760_{—}2, TGME49_{—}016180_{—}1, TGME49_{—}016380_{—}13, TGME49_{—}016380_{—}5, TGME49_{—}021310_{—}9, TGME49_{—}023540_{—}10, TGME49_{—}023540_{—}5, TGME49_{—}024190_{—}10, TGME49_{—}024920_{—}4, TGME49_{—}025320_{—}5, TGME49_{—}026020_{—}8, TGME49_{—}026110_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044040_{—}8, TGME49_{—}044080_{—}3, TGME49_{—}044280_{—}1, TGME49_{—}045500_{—}3, TGME49_{—}046330_{—}5, TGME49_{—}046340_{—}2, TGME49_{—}047370_{—}4, TGME49_{—}047370_{—}9, TGME49_{—}048200_{—}3, TGME49_{—}048670_{—}5, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057080_{—}9, TGME49_{—}057520_{—}1, TGME49_{—}058390_{—}1, TGME49_{—}058980_{—}1, TGME49_{—}059200_{—}2, TGME49_{—}061740_{—}1, TGME49_{—}062920_{—}5, TGME49_{—}063560_{—}6, TGME49_{—}064740_{—}4, TGME49_{—}066760_{—}1, TGME49_{—}067350_{—}1, TGME49_{—}068590_{—}4, TGME49_{—}068590_{—}9, TGME49_{—}070220_{—}3, TGME49_{—}070250_{—}1, TGME49_{—}070250_{—}2, TGME49_{—}072290_{—}1, TGME49_{—}073380_{—}3, TGME49_{—}074060_{—}5, TGME49_{—}074190_{—}2, TGME49_{—}078660_{—}9, TGME49_{—}085240_{—}1, TGME49_{—}085240_{—}3, TGME49_{—}086120_{—}1, TGME49_{—}086450_{—}1, TGME49_{—}088400_{—}9, TGME49_{—}088500_{—}5, TGME49_{—}089380_{—}3, TGME49_{—}089730_{—}4, TGME49_{—}090580_{—}5, TGME49_{—}090870_{—}5, TGME49_{—}090950_{—}5, TGME49_{—}092220_{—}1, TGME49_{—}095650_{—}4, TGME49_{—}097240_{—}6, TGME49_{—}099060_{—}4, TGME49_{—}099060_{—}6, TGME49_{—}100060_{—}2, TGME49_{—}100310_{—}7, TGME49_{—}101270_{—}10, TGME49_{—}105020_{—}9, TGME49_{—}105270_{—}2, TGME49_{—}105510_{—}3, TGME49_{—}105510_{—}5, TGME49_{—}109910_{—}2, TGME49_{—}112600_{—}3, TGME49_{—}113020_{—}8, TGME49_{—}114850_{—}4, TGME49_{—}118460_{—}3, TGME49_{—}16180_{—}1, TGME49_PP2C-hn, TGME49_TLN_{—}1, and fragments thereof.

In another embodiment of the invention, the carrier is a pharmaceutically acceptable carrier, and the composition is formulated as a vaccine. In such embodiments the vaccine may comprise a single T. gondii antigen, however it is generally preferable that the vaccine comprises multiple (e.g., at least two, four, or six) antigens. Depending on the particular T. gondii-induced disease or disorder, it is contemplated that the T. gondii antigens, or fragments thereof, are at least partially purified and/or recombinant.

In another embodiment, immunodominant antigens according to the inventive subject matter may also be employed to generate an antibody preparation that can be used as passive vaccination for therapeutic treatment of toxoplasmosis. In preferred embodiments, such vaccines are subunit vaccines or attenuated live recombinant vaccines. For example, the immunodominant antigens presented herein may be employed in the manufacture of a vaccine that comprises at least one, and more typically at least two of the immunodominant antigens encoded by nucleic acids according to SEQ ID NO:1 to SEQ ID NO:100 or fragments thereof. In a preferred embodiment contemplated vaccines can include between one and five, or at least six, and even more antigens, of which at least one of the antigens is an immunodominant antigen. It should be appreciated that vaccines may be produced that predominantly, or even exclusively, comprise immunodominant antigens characteristic of a single parameter. For example, a vaccine may comprise immunodominant antigens that are characteristic for a population that has an acute infection. Alternatively, the sequences according to SEQ ID NO:1 to SEQ ID NO:100, or fragments thereof, may also be employed as DNA vaccines, or comprise part of an in vivo expression system that triggers an immune response against an in vivo produced recombinant antigen or fragment thereof.

With respect to suitable formulations of vaccines, it should be recognized that all known manners of producing such vaccines are deemed appropriate for use herein, and a person of ordinary skill in the art will be readily able to produce such vaccines without undue experimentation (see e.g., “Vaccine Adjuvants and Delivery Systems” by Manmohan Singh; Wiley-Interscience (Jun. 29, 2007), ISBN: 0471739073; or “Vaccine Protocols” (Methods in Molecular Medicine) by Andrew Robinson, Martin P. Cranage, and Michael J. Hudson; Humana Press; 2 edition (Aug. 27, 2003); ISBN: 1588291405). Therefore, suitable vaccines may be formulated as injectable solutions, or suspensions, intranasal formulations, transdermal or oral formulations.

Additionally, it is contemplated that antigens identified herein may also be employed to generate (monoclonal or polyclonal) antibodies or fragments thereof (e.g., F(ab)′. F(ab)′₂, Fab, scFv, etc.) or other binding species, such as aptamers, that can then be employed in a diagnostic test that directly detects the presence of T. gondii antigens in blood, blood derivatives or other body fluid of a patient where the antigen is present in the patient. It should be appreciated that such an antigen may be associated with the cells of the pathogenic organism, in association with components of the pathogenic organism, complexed with a molecule or cell of the patient, or be in free, uncomplexed form. Most preferably, the antigens are immunodominant and/or serodiagnostic antigens as presented herein. For example, suitable tests can include those in which one or more labeled antibodies are used to detect the presence of the antigen in bodily fluid where the antigen has been captured (specifically or in combination with other proteins) and immobilized on a carrier. There are numerous antigen detection methods known in the art and all of the known formats are deemed suitable for use herein. In some embodiments the carrier may be a solid carrier, and the plurality of T. gondii antigens is disposed on the carrier in an array. It is further contemplated that the antigens or fragments thereof may be in crude expression extracts, in partially purified form (e.g., purity of less than 60%), or in highly purified form (purity of at least 95%). The antigens in such arrays may be recombinant or native. Alternatively, solid phases need not be limited to planar arrays, but may also include fluid suspension arrays, beads, columns, testing dipstick formats, etc.

The inventors have discovered numerous T. gondii antigens that were capable of triggering antibody reactivity from a variety of stages of T. gondii infection. Antigens according to the inventive subject matter were presented herein, and it is contemplated that such antigens can be used by themselves, or more preferably, in combination with other antigens in the manufacture of a diagnostic devices, therapeutic compositions, and vaccines. The compositions, vaccines, diagnostic tests, etc., described herein may be used for both human and veterinary use.

### EXAMPLES

Serum Samples: Serum samples were classified into four groups. Group 1 was composed of seronegative individuals from Turkey with no known history of T. gondii infection. Group 2 was composed of recently acute patients' sera collected during an outbreak of toxoplasmosis. Sera were collected from these patients 1-2 weeks after the onset of symptoms. Group 3 was composed of patients with chronic infections that had persisting IgM antibodies and a high IgG avidity index. Group 4 was composed of patients with chronic infections that were negative for IgM antibodies and that had a high IgG avidity index.

IgG immunofluorescence Assay (IFA): IFA was performed by coating slides with HeLa cell culture and BALB/c derived T. gondii RH Ankara strain tachyzoites. Slides were then probed with anti-Toxoplasma IgG positive patient serum samples at dilutions of 1/16, 1/64, 1/128, 1/256, 1/512 and 1/1024 for 30 minutes at 37° C., and washed 3 times with PBS. The slides were then probed with anti-Human IgG antibody conjugated with fluorescein (Biomerieux, France) at a 1/1,250 dilution for 30 minutes at 37° C. Slides were washed and examined under an immunofluorescence microscope (Olympus, U.S.A.) for quantification of fluorescent parasites. Sera that retained activity over 1/16 dilution were considered seropositive.

Enzyme Linked Immunosorbent Assay (ELISA). Antigen preparation: Antigen was prepared from T. gondii RH Ankara strain tachyzoites obtained from peritoneal exudates of infected BALB/c mice. Tachyzoites were centrifuged at 500×g for 5 minutes and quantified in the supernatant using a haemocytometer. This supernatant was centrifuged for 10 minutes at 3000×g and the pellet washed 3 times with PBS (pH 7.4). The pellet was resuspended in 1% SDS in distilled water and subjected to several cycles of freezing and thawing in order to lyse the cells. The resulting lysate was centrifuged at 14,000×g for 15 minutes and the supernatant containing the antigen suspension was passed through 0.22 m filter (Macherey-Nagel, Germany).

ELISA: Wells of a flat-bottom, high-binding microwell plate (Costar, U.S.A.) were coated with 100 μl of antigen suspension containing the equivalent of 1×10⁵ lysed tachyzoites. Plates were incubated for 1.5 hour at room temperature (RT). Next, serum samples for IgG ELISA (diluted 1/256) and for IgM ELISA (diluted 1/64) were added to the wells, incubated for 1 h at room temperature and washed 3 times with PBS. Serum samples were diluted in a blocking buffer comprised of 0.5% casein in PBS, pH 7.5. IgG ELISA wells were probed with recombinant protein G (Zymed, USA) conjugated with peroxidase at a dilution of 1/50,000; IgM ELISA wells were probed with anti-Human IgM (Sigma, Germany) conjugated with peroxidase at dilution of 1/5,000. Probes were incubated for 30 min at room temperature. Thereafter, peroxidase activity resulting from bound antibodies were visualized after adding 3,3′,5,5′ tetramethylbenzidine (TMB) substrate. Reactions were stopped by adding 75 μl of 2 N sulfuric acid and the results quantified in a microwell plate reader (Bio-Tek ELx808, U.S.A.) at 450 nm. Samples were considered positive if the absorbance value (AV) of the serum samples exceeded the mean AV+7S.D. (for IgG ELISA) and AV+5S.D. (for IgM ELISA) of the negative control serum samples.

IgM capture ELISA: A commercially available IgM capture ELISA kit (Radim Diagnostics, Italy) was used according to the manufacturer's instructions. Controls provided in the kit and the serum samples were diluted to 1/100 in the provided sample diluent and added to a microwell plate pre-coated with monoclonal anti-human IgM antibody to capture serum IgM. The plate was incubated for 1 h at 37° C. and washed 4 times with PBS containing 0.05% Tween-20 (PBS-T). Each well was probed with lyophilized inactivated Toxoplasma antigen reconstituted using a solution of monoclonal anti-Toxoplasma antibody conjugated with biotin, incubated for 1 hr at 37° C. and washed 4 times with PBS-T. After incubation with a peroxidase-conjugated streptavidin at 37° C. for 30 min the microwell plate was washed 4 times in PBS-T and bound antibodies visualized using a TMB substrate at room temperature for 15 min. Reactions were stopped and quantified as above. The presence or absence of anti-Toxoplasma IgM was defined against the AV of the cut-off control supplied in the kit.

IgG avidity assay: Flat bottom high binding microwell plates (Costar, U.S.A.) were coated with tachyzoite lysate as described for the IgG ELISA above. Next, serum samples diluted to 1/256 in blocking buffer were added to a first and a second set of wells and incubated for 15 min at room temperature. 6M urea in blocking buffer was added to the first set of wells and blocking buffer without urea was added to the second set of wells. After incubation for 15 min at room temperature each well was washed 3 times with PBS and probed with recombinant protein G-peroxidase conjugate (Zymed, U.S.A.) at a dilution of 1/50,000 for 15 min at RT. Thereafter, bound antibodies were visualized using TMB substrate and stopped as above. The avidity index (AI) was expressed as a percentage using the following formula: (absorbance value_{first set of wells}/absorbance value_{second set of wells})×100. Sera associated with early infection (<3-4 months) typically had an AI<20%. Sera associated with late infection (>6 months) typically had an AI>30%, whereas between 20-30% was considered borderline. A serum sample with a low AI that was also positive by IgM capture ELISA was classified as an infection occurring within the previous 3-4 months (i.e. recent, acute infection). Samples with a high AI and a positive IgM capture ELISA result were classified as chronic/IgM persisting, whereas samples with a high AI and a negative IgM capture ELISA result were classified as chronic.

Microarray fabrication and probing: Proteome microarrays were fabricated by PCR amplification of coding sequences in genomic DNA, followed by insertion of amplicons into a T7 expression vector by homologous recombination, and expression in coupled transcription-translations in vitro (IVTT) prior to printing onto microarrays. Use of cDNA as the PCR template may underrepresent genes expressed at low levels in vivo. For this reason genomic DNA and amplified exons were used separately. This strategy has been described previously by Doolan et al (Doolan, D. L., Mu, Y., Unal, B., Sundaresh, S., Hirst, S., Valdez, C., Randall, A., Molina, D., Liang, X., Freilich, D. A., Oloo, J. A., Blair, P. L., Aguiar, J. C., Baldi, P., Davies, D. H., and Felgner, P. L. (2008) Profiling humoral immune responses to P. falciparum infection with protein microarrays. Proteomics 8, 4680-4694), which is hereby incorporated in its entirety. PCR primer were designed based on the genomic sequence of type II strain ME49 of T. gondii, which was obtained from the Toxoplasma Genomics Resource (http://toxodb.org/toxo/); a sequential bioinformatic filtering strategy (described below) was applied to prioritize genes targeted for cloning. Custom PCR primers were designed to amplify 2000 exons. The PCR primers comprised 20 bp of exon-specific sequence with 20 bp of adapter sequences, and were used in PCR reactions with 20 ng of genomic DNA. Genomic DNA was obtained from type II Prugniaud strain T. gondi parasites that were freshly lysed from monolayers of human foreskin fibroblasts and extracted using the Wizard Genomic Purification Kit (Promega, Wisconsin) following the manufacturer's instructions. For genes larger than 3 kb additional primer pairs were designed to amplify overlapping fragments of 3 kb each. PCR primers were also designed to amplify complete genes RON5, ROP13, PP2C-hn, PP2C2, 002200, RON4, Toxolysin-1 putative rhoptry metalloprotease, ISP2 and ISP1 from plasmids encoding cDNAs. The adapter sequences, which are incorporated into the termini flanking the amplified gene, are homologous to the cloning site of a linearized T7 expression vector and allow PCR products to be cloned by in vivo homologous recombination in competent DH5α cells. The resulting protein incorporates an ATG translation start codon, a 5′ polyhistidine epitope, a 3′ influenza hemagglutinin epitope and a T7 terminator. For cloning, PCR products were mixed with a linearized expression vector and used to transform super-competent DH5-alpha cells to kanamycin resistance. DNA was purified from the overnight cultures without prior colony selection using QIAprep 96 Turbo Miniprep Kits from Qiagen (Netherlands).

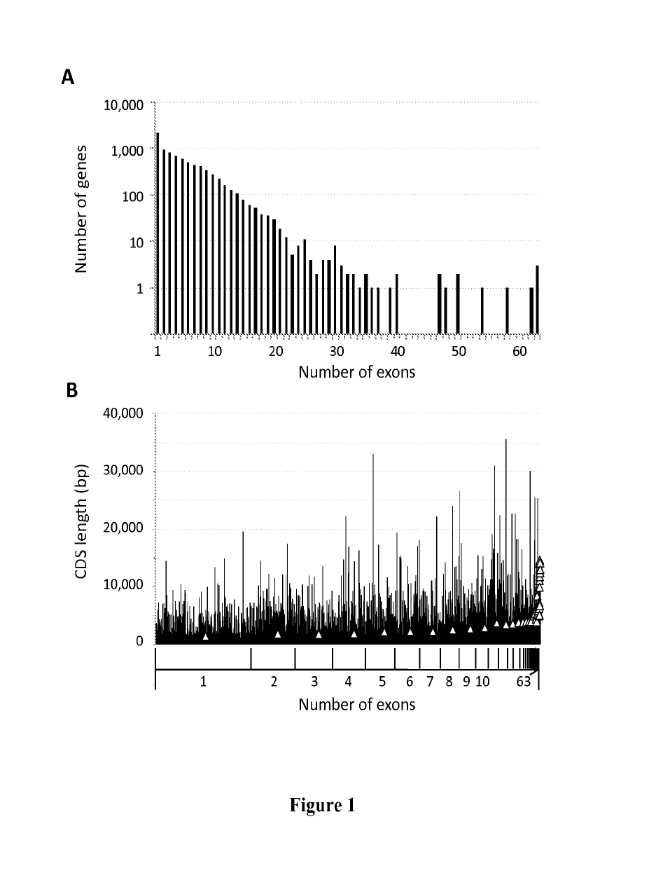

Chip fabrication: The Toxoplasma Genomics Resource or ‘ToxoDB’ (http://toxodb.org/toxo/) lists 8,155 genes in the T. gondii genome, comprising a total of 43,010 exons. The genes have varying numbers of exons, ranging from 1 (n=2,135 genes) to 63 (n=3 genes) (see FIG. 1A) and have lengths varying from 71 to 35,589 bps. There is a general trend for the longer genes to have more exons (see FIG. 1B). To produce the chip array the number of exons was reduced to approximately 2,000 using a bioinformatic filtering process based on antigenic features seen in other proteome-wide serological screens of bacteria. Firstly, genes lacking a mass spectroscopy profile were excluded to enrich for functional genes. Gene Ontology (GO) annotation and “product description” from ToxoDB was then used to identify proteins belonging to the categories of ‘outer membrane’, ‘heat shock protein’, ‘chaperone’, ‘transport protein’, ‘integral membrane protein’, ‘transmembrane protein’, ‘lipoprotein’, or ‘virulence associated protein’. This gave a list of 1,059 genes (6,829 exons), of which 400 exons lacked coding sequence and were excluded. An additional 3,716 exons below 200 bp were also excluded. The remaining 2,705 exons (from 952 genes) were then subjected to high throughput cloning and expression for microarray printing.

Array fabrication: An array chip (“TG1”) comprising the first 1,357 exon products (from 615 genes) amplified from T gondii Prugniaud strain, which ranged from 67 to 158 amino acids in length was fabricated. This represents 50% of the target number of 2,705 exons. Purified minipreparations of DNA were expressed in a commercial E. coli based in vitro transcription/translation expression system (RTS-100 from Roche, Germany). Ten microliter reactions were set up in sealed 384 well plates and incubated for 16 hours at 24° C. on a platform shaker at 300 rpm. A protease inhibitor cocktail (Complete, Roche, Germany) and Tween-20 at a final concentration of 0.05% were added prior to printing. The RTS-100 reaction products were printed in singlicate without further purification onto 2-pad nitrocellulose-coated FAST slides (Whatman, United Kingdom) using a Gene Machine OmniGrid Accent microarray printer (Digilabs Inc., Massachusetts) in 4×4 sub-array format, with each sub-array comprising 108 spots. Each sub-array included multiple negative control spots comprising mock RTS reactions performed without a DNA template. Each sub-array included positive control spots of 4 serial dilutions of mouse, rat, and human whole IgG and 2 serial dilutions of human IgM and mouse IgM. These positive and negative controls were used to normalize data from different arrays. Four serial dilutions of purified recombinant Epstein-Barr virus nuclear antigen-1 (EBNA-1, DevaTal, Inc., Hamilton N.J.), which is recognized by the majority of humans, were also included to serve as an indicator of serum quality. Protein expression for each spot on the array was verified using antibodies to N- and C-terminal polyhistidine and hemagglutinin epitope tags. This confirmed 93% of the expression products were detected by at last one of the epitope tag antibodies.

Expression detection: Expression in each spot of the microarray was detected using anti-tag antibodies directed to the N-terminal poly-His (clone His-1, Sigma) and the C-terminal HA (clone 3F10, Roche) tags engineered into each protein. Arrays were first incubated for 30 minutes in Protein Array Blocking Buffer (Whatman, United Kingdom) at room temperature and then probed for 1 hour with anti-tag antibodies diluted 1/1,000 in blocking buffer. The slides were then washed 6× in tris(hydroxymethyl)aminomethane (Tris)-buffered saline containing 0.05% (v/v) Tween 20, (T-TBS) and incubated with appropriate biotinylated secondary antibodies (Jackson ImmunoResearch, Pennsylvania). After washing the slides 6 times in T-TBS, bound antibodies were detected by incubation with streptavidin-conjugated SureLight® P-3 (Columbia Biosciences, Maryland). The slides were then washed three times in T-TBS followed by TBS, and dipped in distilled water prior to air drying by brief centrifugation. Slides were scanned in a Perkin Elmer ScanArray confocal laser scanner (Perkin Elmer, Massachusetts) and data acquired using ScanArrayExpress software.

Probing with human sera: Serum samples were diluted to 1/200 in Protein Array Blocking Buffer supplemented with E. coli lysate (Antigen Discovery, Inc., California) at a final concentration of 10 mg/ml protein, and incubated at 37° C. for 30 minutes with constant agitation prior to application to the arrays. Arrays were incubated in Protein Array Blocking Buffer for 30 min and probed with the pretreated sera overnight at 4° C. with gentle rocking. Arrays were then washed in T-TBS six times and incubated with biotinylated anti-human IgG H+L (Jackson Immuno Research, Pennsylvania) diluted 1/400 in Protein Array Blocking Buffer. After washing the slides three times in T-TBS followed by three washes in TBS, bound antibodies were visualized as described above.

Data analysis and statistical treatment: Raw data were collected as the mean pixel signal intensity data for each spot on the array. To stabilize variance of the raw data, a variant of the log-transformation (asinh) was used, and negative control (no DNA) and positive control (IgG) spots were used to normalize the data using the “VSN” package in R from the Bioconductor suite (http://Bioconductor.org/). P-values of the normalized data were calculated by comparing signals between groups of donors using a Bayes-regularized t-test adapted from Cyber-T (http://cybert.ics.uci.edu/) for use with protein arrays. To account for multiple test conditions, p-value adjustments were calculated using the Benjamini-Hochberg method (Benjamini, Y., and Hochberg, Y. (1995) Controlling the false discovery rate: a practical and powerful approach to multiple testing. J. Roy. Stat. Soc., B 57, 289-300). Reactive antigens were defined as positive when the normalized signal intensity was greater than the mean+4SD of the average ‘no DNA’ control spots. Discriminatory antigens were those having a Benjamini-Hochberg adjusted Cyber T p-value <0.05. Multiple antigen classifiers were derived using support vector machines (SVMs). The “e1071” and “ROCR” packages in R were used to train the SVMs and to produce receiver operating characteristic curves, respectively. To assess functional enrichment significance, computational predictions of signal peptides and transmembrane domains were obtained from the toxoDB database. Predictions of subcellular localizations were made using WoLF pSort (Horton, P., Park, K. J., Obayashi, T., Fujita, N., Harada, H., Adams-Collier, C. J., and Nakai, K. (2007) WoLF PSORT: protein localization predictor. Nucleic Acids Res 35, W585-587.). P values for enrichment statistical analysis were calculated using Fisher's exact test in the R environment.

IgG profiles defined by microarray correlate with conventional IgG assays: As noted above, sera were classified into four groups according to the panel of four conventional antibody assays. These data are summarized in Table 1.

TABLE 1

| | | | | | Capture IgM | |
|---|---|---|---|---|---|---|
| | Number of | IgG IFA | IgG ELISA | | (median | |
| | sera | (median | (median | IgM ELISA | Absorbance | Avidity Index^{a} |
| | probed | titer) | titer) | (% positive) | Value) | (Avg + SD) |
| 1. Negative | 27 | Negative | Negative | Negative | Negative | Low |
| | | (<64) | (<64) | (ND) | (<215) | (ND) |
| 2. Acute | 27 | Positive | Positive | Positive | Positive | Low |
| | | (4096) | (2048) | (100%) | (1923) | (11.2 + 3.6) |
| 3. Chronic/IgM | 27 | Positive | Positive | Positive | Positive | High |
| Persisting | | (128) | (2048) | (ND) | (1053) | (50.3 + 13.2) |
| 3. Chronic | 27 | Positive | Positive | Negative | Negative | High |
| | | (64) | (512) | (ND) | (164) | (62.3 + 10.3) |

Avidity index values <20 indicative of infection less than 3-4 months; 20-30 considered borderline; >30 indicative of infection more than 6 months. Group 1 was composed of individuals from Turkey with no known history of T. gondii infection and who were seronegative by all conventional assays. Group 2 comprised recently acute cases from a 2002 Turkish outbreak. All sera were collected within approximately 2-3 weeks of the outbreak occurring, and each donor had clinical symptoms consistent with a recent infection. These individuals were IgG positive/IgM positive and low IgG avidity in conventional tests. Group 3 comprised chronic/IgM persisting infections, which were characterized as IgG positive/IgM positive and high IgG avidity in conventional tests. Although having the high avidity IgG, a hallmark of chronic infection, the persisting IgM response normally precludes them from being classified as regular chronic cases. Similarly, the high avidity excludes them from the acute group. Group 4 was true chronic infections characterized by being IgG positive, with high avidity, but IgM negative.

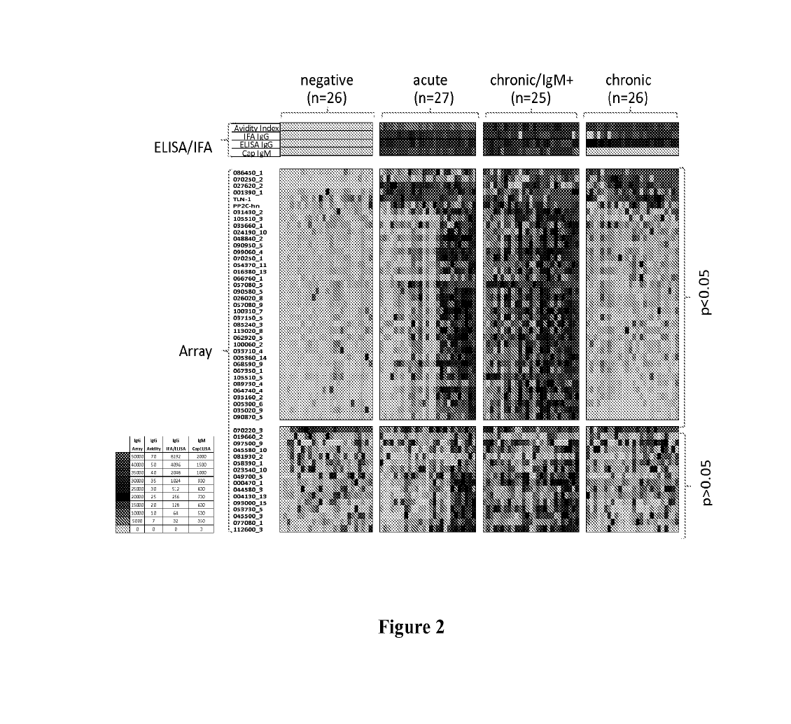

IgG antibody profile: FIG. 2 shows a ‘heat map’ of relative signal intensity for a T. gondii chip array probed for IgG following exposure to patient sera. The data obtained from the conventional IgG and IgM assays are also represented above the heat map for comparison. The reactive antigens (listed on the vertical axis) are separated into discriminatory (BH-corrected p value <0.05; n=38) and non-discriminatory (BH-corrected p value >0.05; n=16) when the negative control population was compared to each of the three infected populations by T test. Reactivity by the seronegative group was minimal on the array, whereas the acute population showed strong IgG reactivity. Interestingly, 3 antigens were recognized by the majority of the donors in the acute stage, (027620_{—}2, dense granule protein GRA2; 001390_{—}1, small secreted protein, and the putative rhoptry metalloproteinase TLN-1), consistent with the IgG ELISA and IFA. It is notable that all three of these are secreted proteins. The remaining reactive antigens (n>30) were only recognized by less than half of the acute donors. These donors may represent the earliest acute infections where the maximal profile is yet to be attained. The chronic/IgM persisting group was characterized by the broader antibody profile, as was observed in approximately half of the acute infections. Thus, a second characteristic feature of the chronic/IgM persisting phenotype, in addition to the persistence of IgM itself, is a broad IgG antibody specificity profile. Unexpectedly, the true chronic cases, which were diagnosed on the basis of high IgG/high avidity but no IgM, were seen to have a significantly narrower IgG specificity profile on the array. Overall, the breadth of the IgG profile appears to increase to a peak in the chronic/IgM persisting stage, but decreases in the true chronic population.

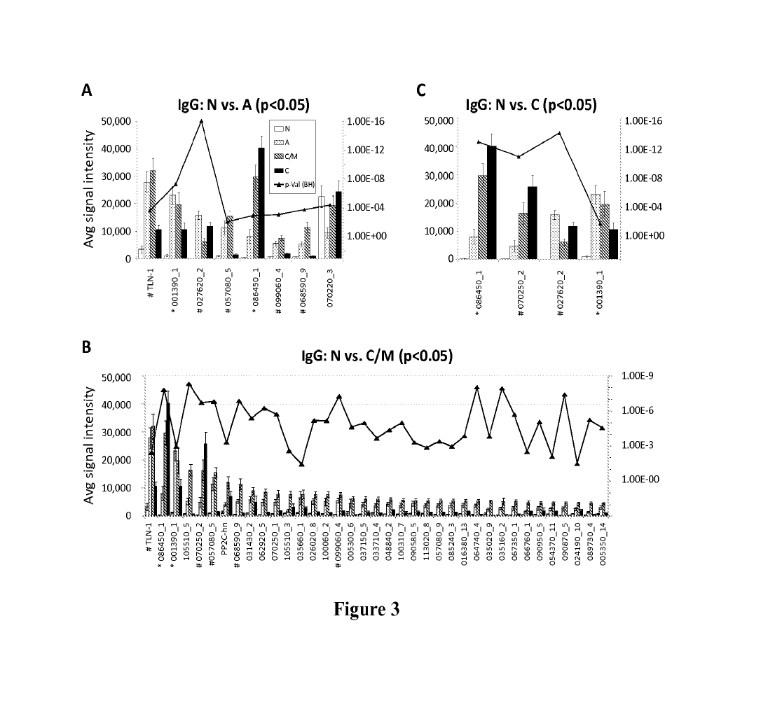

IgG responses with potential diagnostic utility: IgG target antigens that provide the best discrimination between uninfected individuals and each of the three infected states are shown in FIG. 3. Eight antigens are identified that discriminate negative versus acute cases (FIG. 3A). Seven of these showed minimal reactivity to negative controls, stronger reactivity in acute and chronic/IgM persisting groups, but lower reactivity in the true chronic stage. An exception was antigen 086450_{—}1, whose average signal continued to rise throughout time course of the infection. The largest number of discriminatory antigens (n=37) was found when comparing negative to chronic/IgM persisting samples, i.e., when the response peaks, although the majority of these antigens had weak signals (FIG. 3B). Finally, four antigens discriminated chronic from negative cases (FIG. 3C). Antigen 086450_{—}1 and 001390_{—}1 were discriminatory in all three stages of infection, and antigen 070250_{—}2, appeared to react more strongly in chronic populations than in acute populations.

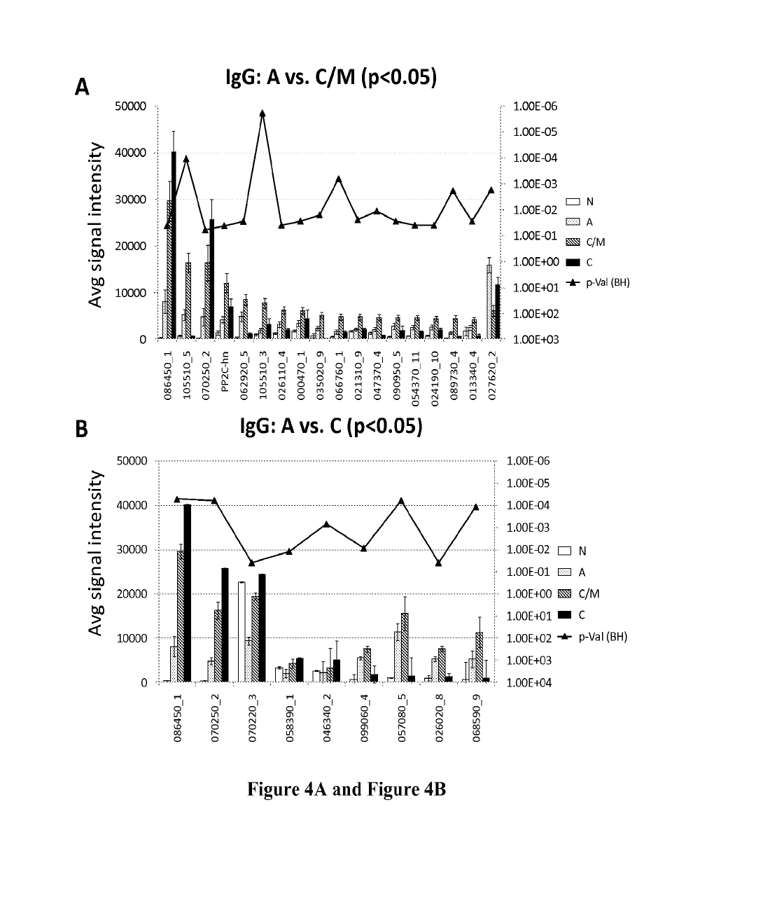

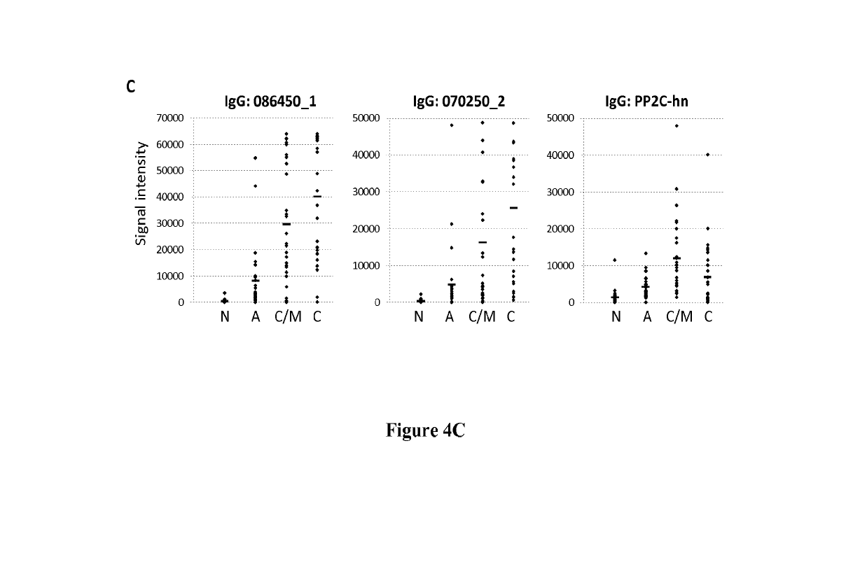

Antigen characterization: Antigens characterized by “ascending” IgG responses (i.e., low signal in acute but high in chronic/IgM persisting and chronic) may be of particular use in excluding a diagnosis of acute infection. To better identify such antigens, samples form acute stage infections were compared with samples from chronic/IgM persisting infections and chronic infections (see FIGS. 4A and 4B, respectively) using T tests. Responses to only two of the 18 discriminatory antigens in FIG. 4A were of the ascending type (exons 086450_{—}1 and 070250_{—}2). The response to antigen PP2C-hn also remained relatively high in chronic infection. The remaining 15 responses peaked in the chronic/IgM persisting stage and fell to almost background levels in the chronic stage. In FIG. 4B is shown a similar analysis comparing acute infections to true chronic infections. Responses to 5 antigens were of the ascending type, whereas the remaining 4 peaked in chronic/IgM persisting infections and were lower in true chronically infected individuals. Average responses to two ascending antigens (exons 086450_{—}1 and 070250_{—}2 again) were low in acute infection, while high in both chronic/IgM persisting infections and true chronic infection. Three discriminatory antigens (exons 086450_{—}1, 070250_{—}2 and PP2C-hn) are shown in dot plots of (FIG. 4C) to display the response at the level of the individual donor.

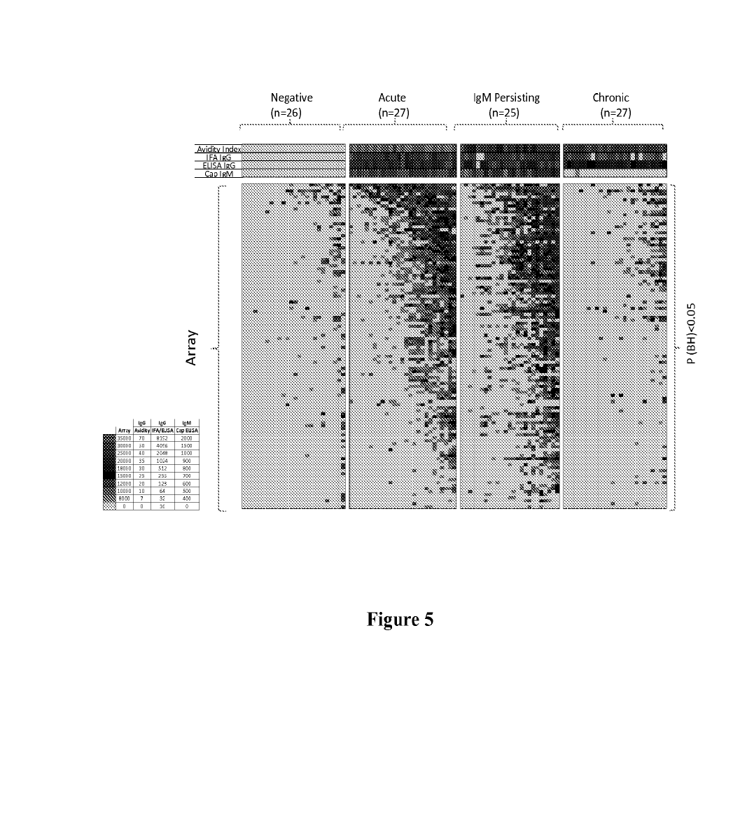

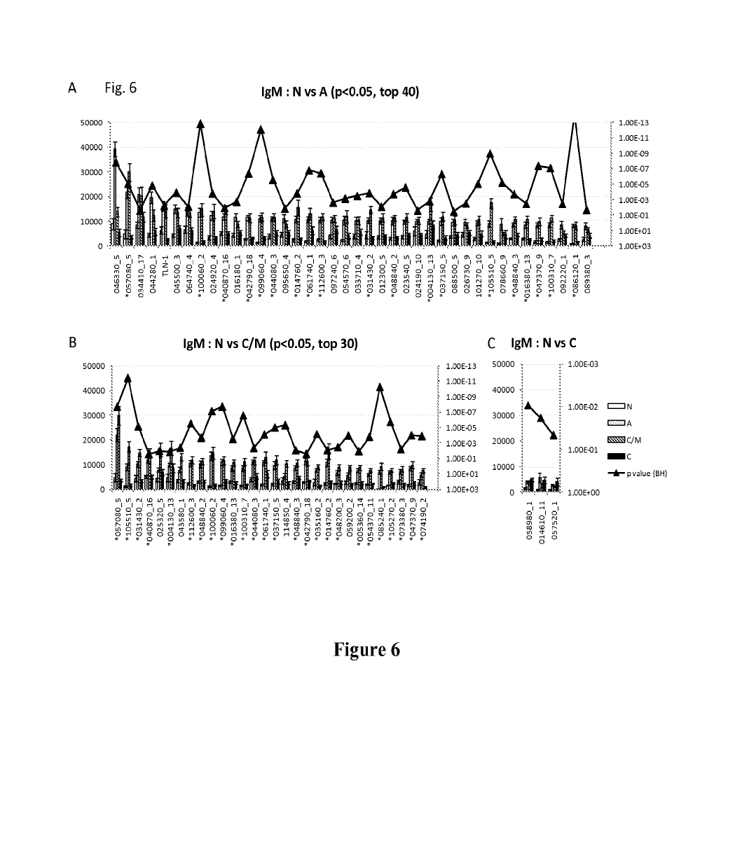

Human IgM profiles: FIG. 5 shows a ‘heat map’ of relative signal intensity for a T. gondii chip array probed for IgM following exposure to patient sera. There were 108 antigens that discriminated between the negative population and the three infected populations (BH-corrected p value <0.05; FIG. 5). The reactivity found in samples from negative donors was minimal, highest in acute and chronic/IgM persisting infections, and returning to background levels in chronic infections. IgM target antigens that discriminate between naïve controls and each of the three infected states are shown in FIG. 6. There were 93 antigens that could discriminate acute infections from uninfected negative controls (40 of which are shown in FIG. 6A), 60 antigens that could discriminate between chronic/IgM persisting infections from negative controls (30 of which are shown in FIG. 6B), 48 of which could discriminate both from uninfected negative controls, and 3 that could discriminate chronic infections from uninfected negative controls (FIG. 6C).

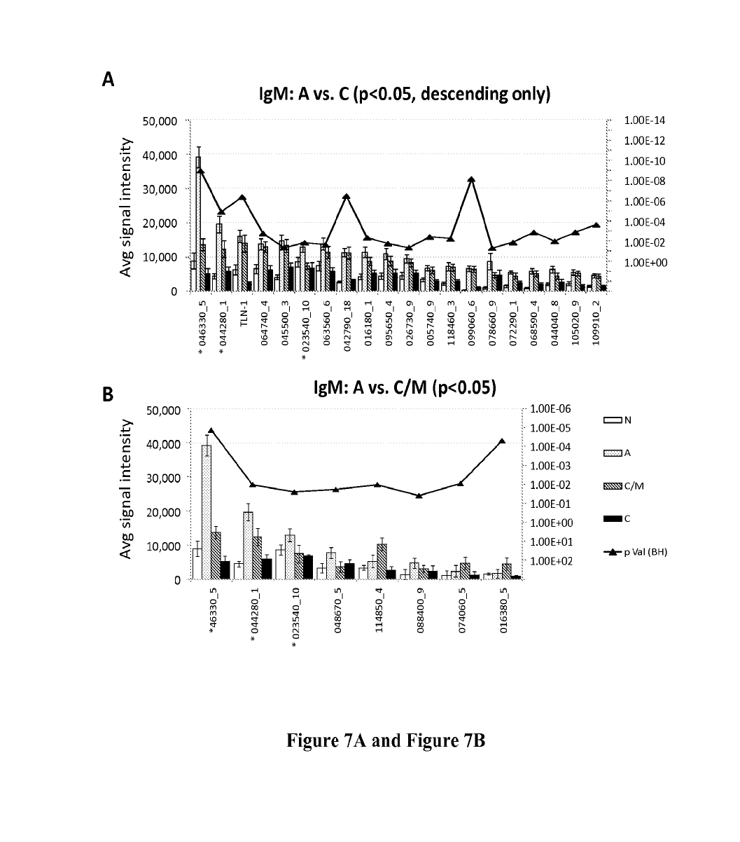

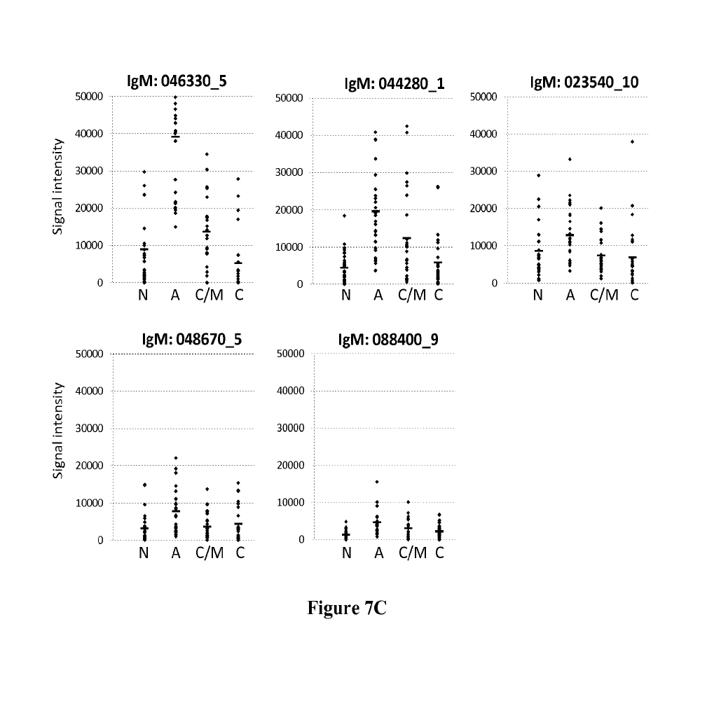

Identification of IgM responses with diagnostic utility: Chip arrays were used to identify specific antigens recognized by IgM in the acute stage of infection whose titers fell in the chronic/IgM persisting and chronic stages (“descending” antigens). Data sets from chronic/IgM persisting infection and chronic infections were combined and the pooled data compared with data from acute infections using BH-corrected T-tests. A total of 91 discriminatory antigens (p<0.05) were found, of which 20 were peaked in the acute stage (see FIG. 7A). However, IgM titers to all but three of these antigens (046330_{—}5, 044280_{—}1 and 023540_{—}10) remained elevated in both the chronic/IgM persisting and acute stages stage and therefore of limited use for diagnosing acute infection. The analysis was repeated comparing IgM in acute infections vs. chronic/IgM infections persisting to determine if additional candidates could be discovered. Eight antigens were discriminatory, (see FIG. 7B), of which five antigens were of the descending type and therefore potentially diagnostic. These 5 antigens are distinct from the three IgG targets shown in FIG. 4C. Since averages indicate an overall trend in a response that may vary between individuals, it is important to know whether this also applies at the individual patient level. Thus, these five descending-type antigens are shown in the dot plots representing individual results in FIG. 7C.

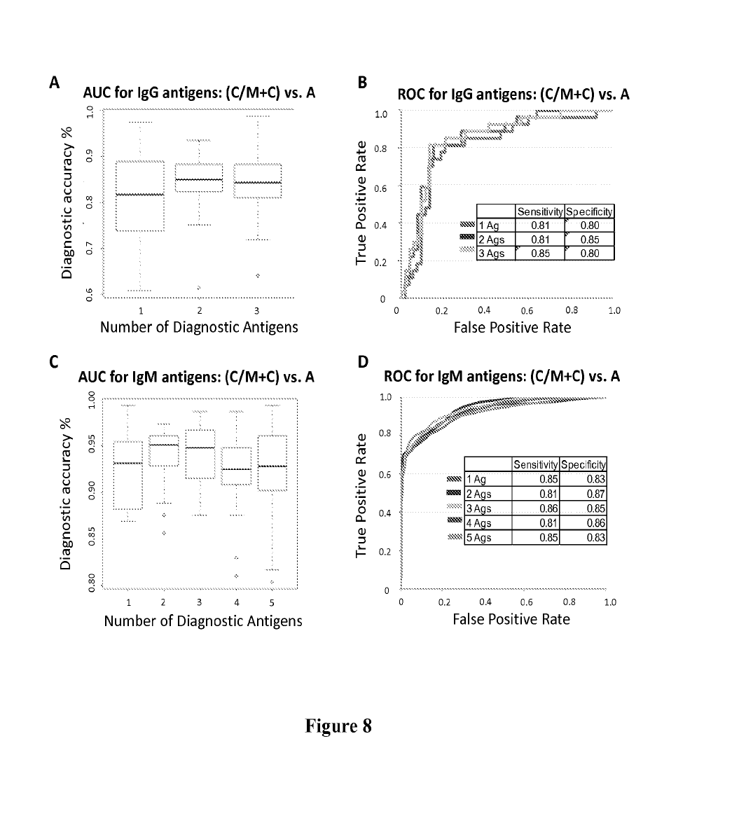

ROC analysis: To assess the accuracy of this collection of antigens in distinguishing acute infection from all chronic infections (chronic and chronic/IgM persisting), cross-validation receiver operating characteristic (ROC) curves and area under the curve (AUC) box plots were generated (see FIG. 8). The candidate serodiagnostic antigens were ranked by decreasing single antigen AUC. The three IgG target antigens, 086450_{—}1, 070250_{—}2, and pp 2C-hn, have AUC values of 0.83, 0.79 and 0.64, respectively, with 086450_{—}1 giving a single antigen discrimination with a sensitivity and specificity of 81% and 80% (see FIGS. 8A and 8B), respectively. Five IgM target antigens, 046330_{—}5, 044280_{—}1, 023540_{—}10, 088400_{—}9 and 048670_{—}5, have AUC values of 0.92, 0.82, 0.79, 0.73 and 0.65, respectively. Exon 046330_{—}5 gave a single antigen discrimination with sensitivity and specificity of 85% and 83%, respectively (see FIGS. 8C and 8D). Kernel methods and support vector machines were used to build linear and nonlinear classifiers. The highest-ranking 1, 2, 3, 4, and 5 antigens on the basis of single antigen AUC were used as input to the classifier. The results were validated with 30 runs of 3-fold cross-validation, and the validation results are averaged over the rounds. This classifier yielded the highest sensitivity and specificity rate of 81% and 85% for the top 2 IgG antigens, with a mean accuracy rate of 85%. While combining the top three antigens increased the sensitivity to 85%, the specificity fell to 80%. For IgM antigens, the combined top 3 produced sensitivity and specificity over 85% with mean AUC of 94%.

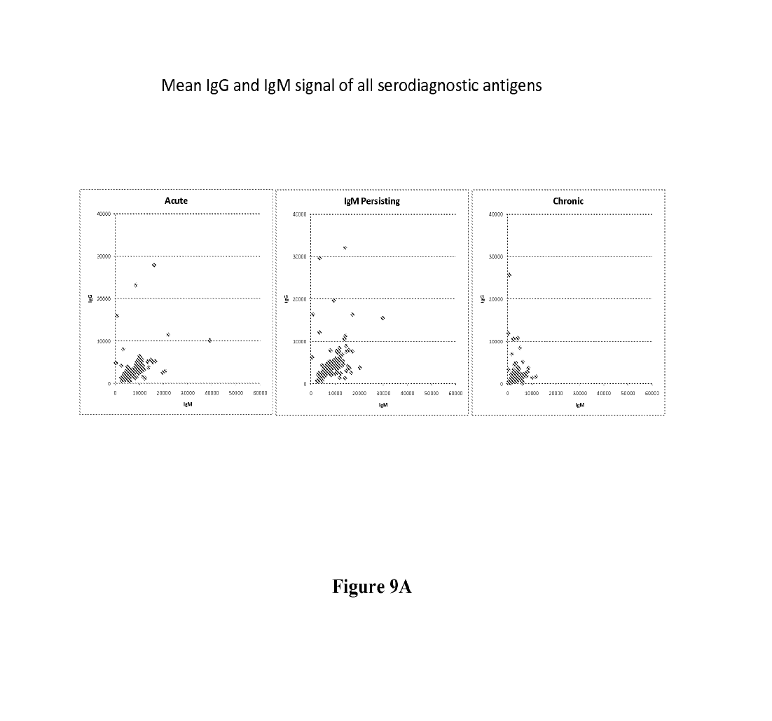

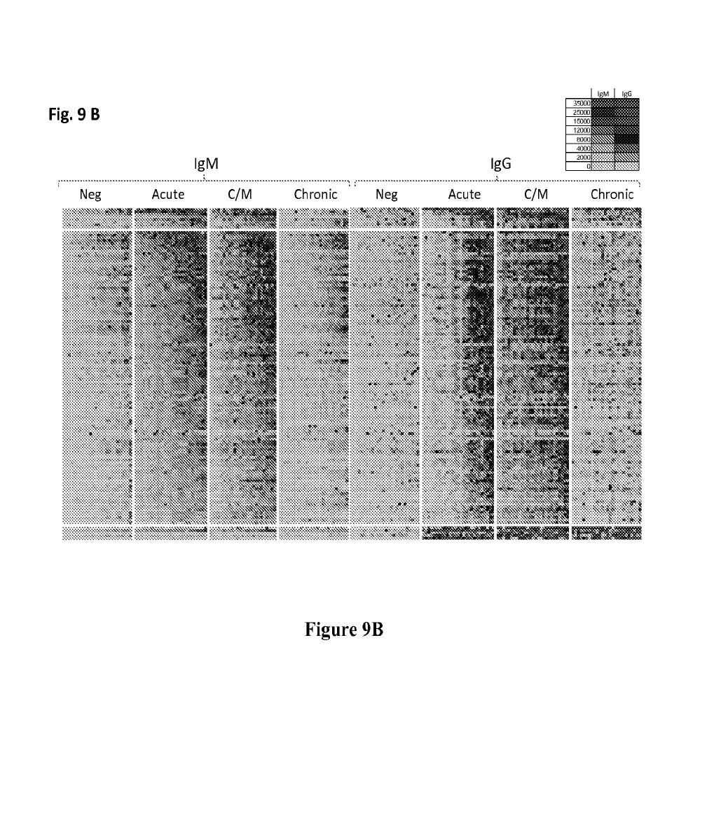

Overlap of IgM and IgG profiles: Class-switching from IgM to other immunoglobulin isotypes is an important component of the maturation of an immune response. It is notable that the number of IgM targets that discriminate between negative uninfected controls and all three stages of T. gondii infection was found to be substantially greater than the number of discriminatory IgG targets (108 and 38, respectively). Scatter plots (see FIG. 9A) of the average IgG and IgM signals in each of three stages of infection illustrate the extent of the overlap; combined, there are a total of 126 different antigens in both IgM and IgG profiles, of which 20 were seen in both, consistent with class switching. In addition, 88 antigens were seen in the IgM profile but not the IgG profile, and a further 18 antigens were seen in the IgG profile but not the IgM profile. This can also be observed in the heat map of signal intensity for chip arrays probed using IgG and IgM from uninfected controls and all three stages of T. gondii infection, as shown in FIG. 9B.

Enrichment analysis: To further characterize the underlying antigenicity of T. gondii enrichment analysis of the discriminating antigens identified above was performed. Antigens were assigned to a Gene Ontology (GO) classification (component, process and function) as defined by ToxoDB. In addition, computational predictions were made for transmembrane domains, signal peptides, isoelectric point (pI), ortholog group information and subcellular localization. The number of reactive discriminatory antigens identified on the array in each classification was divided by the total number of genes the T. gondii genome with this classification to give a figure for fold-enrichment. The significance of enrichment values were also calculated using Fisher's exact test in the R environment. Classifications that are over-represented have values >1 and those under-represented have values <1. A p-value of <0.05 indicated a significant fold-enrichment. It was noted proteins that harbor transmembrane domains were significantly enriched in discriminatory antigens. Interestingly, as the number of predicted transmembrane domains increased from 1 to 10, fold-enrichment also increased from 2.2 to 9.6, with p-values of 4.47E-04 and 1.327E-10, respectively. Conversely, proteins without transmembrane domains were significantly underrepresented (0.6 fold-enrichment; p-value 7.01E-18). Proteins with signal peptides were significantly enriched, as were outer membrane proteins (fold-enrichment of 2.9 and 2.9, respectively, and p values 1.366E-21 and 2.533E-13, respectively). Conversely, proteins that do not have signal peptides were significantly underrepresented (0.5 fold), as were proteins predicted by WoLF pSort to localize in cytosol and nucleus (0.6 fold and 0.4 fold respectively). Findings are summarized in Table 2.

TABLE 2

| | proteins | exons | Exon | Gene | Serodiagnostic | |
|---|---|---|---|---|---|---|
| Predictions | in category | on chip | Hits | Hits | FoldEnrich | p-value |
| TMHMM = 0 | 6656 | 803 | 57 | 53 | 0.6 | 7.010E−18 |
| TMHMM = 1 | 672 | 218 | 21 | 21 | 2.2 | 4.470E−04 |
| TMHMM > 1 | 827 | 346 | 48 | 41 | 3.5 | 1.198E−13 |
| TMHMM >= 5 | 341 | 215 | 37 | 31 | 6.4 | 2.125E−17 |
| TMHMM >= 10 | 103 | 81 | 15 | 14 | 9.6 | 1.327E−10 |
| Signal Peptide | 1760 | 864 | 77 | 72 | 2.9 | 1.366E−21 |
| no Signal Peptide | 6395 | 503 | 49 | 43 | 0.5 | 1.366E−21 |
| WoLF pSort Cytoskeleton | 25 | 1 | 0 | 0 | 0.0 | 1.000E+00 |
| WoLF pSort Cytosol | 1402 | 120 | 13 | 11 | 0.6 | 4.306E−02 |
| WoLF pSort E.R. | 43 | 40 | 1 | 1 | 1.7 | 4.477E−01 |
| WoLF pSort Extracellular | 1213 | 230 | 26 | 24 | 1.4 | 6.008E−02 |
| WoLF pSort Golgi | 3 | 0 | 0 | 0 | 0.0 | 1.000E+00 |
| WoLF pSort Mitochondria | 842 | 202 | 11 | 11 | 1.0 | 1.000E+00 |
| WoLF pSort Nuclear | 3379 | 287 | 18 | 18 | 0.4 | 1.507E−08 |
| WoLF pSort Peroxisome | 26 | 7 | 1 | 1 | 2.8 | 3.013E−01 |
| WoLF pSort Plasma | 1228 | 484 | 56 | 49 | 2.9 | 2.533E−13 |
| membrane | | | | | | |
| WoLF pSort Null | 321 | 17 | 1 | 1 | 0.2 | 1.347E−01 |
| pI 0-5 | 1069 | 108 | 7 | 6 | 0.4 | 0.008 |
| pI 5-7 | 2603 | 569 | 48 | 44 | 1.2 | 0.158 |
| pI 7-9 | 2305 | 491 | 48 | 44 | 1.4 | 0.021 |
| pI 9-14 | 2016 | 199 | 23 | 21 | 0.7 | 0.127 |
| pI null | 162 | 0 | 0 | 0 | 0.0 | 0.176 |
| Ortholog group 4 | 6967 | 1353 | 121 | 113 | 1.2 | 6.036E−06 |
| Other ortholog groups | 1188 | 14 | 2 | 2 | 0.1 | 6.036E−06 |
| Total Proteins | 8155 | 1367 | 126 | 115 | | |

Antigens classified according to GO components: The 115 serodiagnostic IgG and IgM antigens (126 exon hits) identified in this study were analyzed for enrichment against full genome. GO component predictions were obtained from ToxoDB.org. Gene Ontology (GO) classification of reactive discriminating antigens showed that membrane associated proteins were enriched (fold-enrichment of 5.6; p value 4.079E-15). Interestingly, there were 2 antigens that were classified as GO protease complexes, compared to 22 total GO protease complexes in T. gondii genome (6.4 fold-enrichment; p-value 0.038). Proteins not assigned to GO component categories were underrepresented (0.7 fold-enrichment; p value 3.876E-10). Results are summarized in Table 3.

TABLE 3

| GO Component | proteins | exons | Exon | | Serodiagnostic | |
|---|---|---|---|---|---|---|
| predictions | in category | on chip | Hits | Gene Hits | FoldEnrich | p-value |
| GO Cytoskeleton | 28 | 4 | 0 | 0 | 0.0 | 1.000 |
| and organization | | | | | | |
| GO | 19 | 13 | 0 | 0 | 0.0 | 1.000 |
| Chromosome | | | | | | |
| GO ER and | 34 | 41 | 3 | 2 | 4.2 | 0.083 |
| Golgi network | | | | | | |
| GO Intracellular | 272 | 49 | 3 | 3 | 0.8 | 1.000 |
| GO Extracellular | 28 | 5 | 0 | 0 | 0.0 | 1.000 |
| GO | 30 | 10 | 1 | 1 | 2.4 | 0.347 |
| Mitochondria | | | | | | |
| GO Membrane | 381 | 260 | 35 | 30 | 5.6 | 4.079E−15 |
| associated | | | | | | |
| GO Motor | 18 | 18 | 0 | 0 | 0.0 | 1.000 |
| proteins | | | | | | |
| GO Protease | 22 | 7 | 2 | 2 | 6.4 | 0.038 |
| complex | | | | | | |
| GO signal | 8 | 6 | 0 | 0 | 0.0 | 1.000 |
| recognition | | | | | | |
| complex | | | | | | |
| GO Other | 236 | 62 | 5 | 5 | 1.5 | 0.389 |
| Cytoplasm, | | | | | | |
| nucleus or | | | | | | |
| nuclear pore | | | | | | |
| Other GO | 78 | 16 | 0 | 0 | 0.0 | 0.630 |
| components | | | | | | |
| GO component | 7001 | 876 | 77 | 72 | 0.7 | 3.876E−10 |
| Null | | | | | | |
| Total GO | 8155 | 1367 | 126 | 115 | | |
| component | | | | | | |

T. gondii proteins assigned by GO functions are shown in Table 5. Proteins involved in protein binding, catalytic activity, transporter activity, transferase activity were significantly enriched (2.0, 4.0, 5.3, 2.8 fold, respectively). Proteins with enzymatic activity other than kinase activity were enriched at 2.0 fold, and enzyme regulator activity, structural molecule activity and ion channel activity were enriched at 21.5, 9.7 and 7.6 fold, respectively. Interestingly, we identified 2 antigens with GO solute:hydrogen antiporter activity, out of 4 from the genome, leading to 32.2-fold enrichment. There were a total of 5,491 proteins with GO null functions, which was 0.6 fold underrepresented. Proteins involved in nucleotide and nucleic acid binding were also underrepresented at 0.4-fold.

Antigens classified according to GO processes: Table 4 shows T. gondii proteins assigned by GO process classification. Proteins involved in ATP biosynthetic process were significantly enriched (23.3 fold; p value 8.361E-09) among reactive discriminatory antigens. Several proteins involved in transport were also significantly enriched: ion transport, protein transport, vesicle mediated transport, and other transport functions were enriched (7.8, 4.5, 6.9, and 7.0 fold, respectively). Proteins involved in metabolic process, proteolysis, and signal peptide processing were also enriched (3.4, 4.1 and 20.0 fold, respectively). Conversely, proteins not assigned with GO process categories were significantly underrepresented (0.5 fold; p value 3.301E-21).

TABLE 4

| | proteins | | | | Sero- | |
|---|---|---|---|---|---|---|
| GO Process | in | exons | | | diagnostic | |
| predictions | category | on chip | Exon Hits | Gene Hits | FoldEnrich | p-value |
| Cytoskeleton | 11 | 0 | 0 | 0 | 0.0 | 1.000 |
| Choromosome | 11 | 10 | 0 | 0 | 0.0 | 1.000 |
| organization | | | | | | |
| biosynthetic | 132 | 27 | 1 | 1 | 0.5 | 0.728 |
| process | | | | | | |
| Microtubule | 44 | 19 | 0 | 0 | 0.0 | 1.000 |
| based movement | | | | | | |
| and process | | | | | | |
| ATP | 18 | 61 | 9 | 7 | 23.3 | 8.361E−09 |
| biosynthetic | | | | | | |
| process | | | | | | |
| ion transport, | 69 | 83 | 12 | 9 | 7.8 | 1.852E−06 |
| cation transport, | | | | | | |
| proton transport | | | | | | |
| protein transport | 80 | 92 | 7 | 6 | 4.5 | 0.002 |
| Vesicle mediated | 52 | 59 | 7 | 6 | 6.9 | 2.080E−04 |
| transport | | | | | | |
| other transport | 86 | 88 | 10 | 10 | 7.0 | 1.436E−06 |
| metabolic | 366 | 175 | 26 | 21 | 3.4 | 6.003E−07 |
| process | | | | | | |
| oxidation | 61 | 22 | 4 | 3 | 3.0 | 0.081 |
| reduction | | | | | | |
| glycolysis | 19 | 3 | 0 | 0 | 0.0 | 1.000 |
| immune | 1 | 0 | 0 | 0 | 0.0 | 1.000 |
| response, | | | | | | |
| antigen | | | | | | |
| processing and | | | | | | |
| presentation | | | | | | |
| protein catabolic | 56 | 16 | 3 | 3 | 3.2 | 0.066 |
| process | | | | | | |
| transcription | 64 | 2 | 2 | 2 | 1.9 | 0.289 |
| translation | 228 | 30 | 2 | 2 | 0.5 | 0.594 |
| protein | 5 | 12 | 1 | 1 | 12.0 | 0.081 |
| glycosylation, | | | | | | |
| protein | 185 | 15 | 1 | 1 | 0.3 | 0.377 |
| phosphorylation | | | | | | |
| proteolysis | 117 | 73 | 8 | 8 | 4.1 | 0.001 |
| protein folding | 40 | 32 | 1 | 1 | 1.5 | 0.490 |
| cell redox | 36 | 17 | 1 | 1 | 1.7 | 0.454 |
| homeostasis | | | | | | |
| signal | 70 | 51 | 4 | 3 | 2.6 | 0.111 |
| transduction, | | | | | | |
| pathway | | | | | | |
| tRNA | 33 | 13 | 1 | 1 | 1.8 | 0.426 |
| aminoacylation | | | | | | |
| RNA processing, | 83 | 11 | 2 | 2 | 1.4 | 0.404 |
| modification | | | | | | |
| RNA splicing | 2 | 0 | 0 | 0 | 0.0 | 1.000 |
| Signal peptide | 3 | 3 | 1 | 1 | 20.0 | 0.049 |
| processing | | | | | | |
| cell adhesion | 6 | 3 | 0 | 0 | 0.0 | 1.000 |
| DNA | 88 | 10 | 0 | 0 | 0.0 | 0.407 |
| recombination, | | | | | | |
| repair, | | | | | | |
| replication and | | | | | | |
| modification | | | | | | |
| methylation | 15 | 0 | 0 | 0 | 0.0 | 1.000 |
| protein | 20 | 9 | 0 | 0 | 0.0 | 1.000 |
| localization | | | | | | |
| protein | 25 | 3 | 0 | 0 | 0.0 | 1.000 |
| modification | | | | | | |
| process | | | | | | |
| protein | 9 | 0 | 0 | 0 | 0.0 | 1.000 |
| polymerization | | | | | | |
| response to | 8 | 3 | 0 | 0 | 0.0 | 1.000 |
| stress | | | | | | |
| defense response | 2 | 1 | 0 | 0 | 0.0 | 1.000 |
| pathogenesis | 4 | 2 | 0 | 0 | 0.0 | 1.000 |
| regulation of | 17 | 1 | 0 | 0 | 0.0 | 1.000 |
| Rab GTPase | | | | | | |
| activity | | | | | | |
| iron-sulfur | 7 | 1 | 0 | 0 | 0.0 | 1.000 |
| cluster assembly | | | | | | |
| Other GO | 94 | 0 | 0 | 0 | 0.0 | 0.411 |
| process | | | | | | |
| Go Process Null | 6299 | 647 | 54 | 52 | 0.5 | 3.301E−21 |
| | 8466 | 1594 | 157 | 141 | | |

Antigens classified according to GO functions: Reactive discriminating T. gondii proteins assigned by GO functions are shown in Table 5.

TABLE 5

| GO Function | proteins | exons | Exon | Gene | Serodiagnostic | |
|---|---|---|---|---|---|---|
| predictions | in category | on chip | Hits | Hits | FoldEnrich | p-value |
| Cytoskeleton | 3 | 0 | 0 | 0 | 0.0 | 1.000 |
| Nucleic Acid, | 1017 | 269 | 12 | 6 | 0.4 | 0.007 |
| nucleotide binding | | | | | | |
| Protein binding | 289 | 149 | 10 | 9 | 2.0 | 0.046 |
| Ion binding | 371 | 134 | 10 | 9 | 1.6 | 0.191 |
| Other binding | 168 | 117 | 3 | 3 | 1.1 | 0.747 |
| Catalytic Activity | 292 | 136 | 22 | 18 | 4.0 | 5.647E−07 |
| Microtubule motor | 35 | 19 | 0 | 0 | 0.0 | 1.000 |
| activity | | | | | | |
| Transporter | 73 | 69 | 6 | 6 | 5.3 | 0.001 |
| Activity | | | | | | |
| Transferase | 115 | 34 | 5 | 5 | 2.8 | 0.033 |
| activity | | | | | | |
| Kinase activity | 199 | 18 | 1 | 1 | 0.3 | 0.378 |
| Other enzymatic | 919 | 366 | 31 | 29 | 2.0 | 1.914E−04 |
| activity | | | | | | |
| Enzyme activator | 27 | 4 | 0 | 0 | 0.0 | 1.000 |
| activity | | | | | | |
| Enzyme inhibitor | 4 | 3 | 0 | 0 | 0.0 | 1.000 |
| activity | | | | | | |
| Enzyme regulator | 3 | 6 | 1 | 1 | 21.5 | 0.046 |
| activity | | | | | | |
| structural molecule | 20 | 23 | 4 | 3 | 9.7 | 0.003 |
| activity | | | | | | |
| Transcription | 90 | 2 | 2 | 2 | 1.4 | 0.652 |
| factor activity | | | | | | |
| Translation factor | 36 | 7 | 1 | 1 | 1.8 | 0.432 |
| activity | | | | | | |
| Solute:hydrogen | 4 | 13 | 3 | 2 | 32.2 | 0.001 |
| antiporter activity | | | | | | |
| Ion channel, | 17 | 12 | 2 | 2 | 7.6 | 0.028 |
| potassium channel | | | | | | |
| activity | | | | | | |
| Chaperone activity | 4 | 5 | 0 | 0 | 0.0 | 1.000 |
| Structural | 121 | 1 | 0 | 0 | 0.0 | 0.268 |
| constituent of | | | | | | |
| ribosome | | | | | | |
| Hedgehog receptor | 4 | 3 | 1 | 1 | 16.1 | 0.061 |
| activity | | | | | | |
| Protein Kinase | 9 | 0 | 0 | 0 | 0.0 | 1.000 |
| regulator activity | | | | | | |
| Other GO | 22 | 8 | 0 | 0 | 0.0 | 1.000 |
| functions | | | | | | |
| GO function null | 5491 | 566 | 50 | 47 | 0.6 | 1.219E−10 |
| Total GO | 9333 | 1964 | 164 | 145 | | |
| functions | | | | | | |

Proteins involved in protein binding, catalytic activity, transporter activity, transferase activity were significantly enriched (2.0, 4.0, 5.3, 2.8 fold, respectively). Proteins with enzymatic activity other than kinase activity were enriched at 2.0 fold, and enzyme regulator activity, structural molecule activity and ion channel activity were enriched at 21.5, 9.7 and 7.6 fold, respectively. Interestingly, we identified 2 antigens with GO solute:hydrogen antiporter activity, out of 4 from the genome, leading to 32.2-fold enrichment. There were a total of 5,491 proteins with GO null functions, which was 0.6 fold underrepresented. Proteins involved in nucleotide and nucleic acid binding were also underrepresented at 0.4-fold.

Thus, specific embodiments and applications of T. gondii antigen and antibody compositions and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. An antigen composition comprising:
an antibody reactive antigen associated with a carrier;
wherein the antigen has quantified and known relative antibody reactivity with respect to sera of a population affected by Toxoplasma gondii;
wherein the antigen has a known association with a disease parameter; and wherein the antigen is selected from the group consisting of:
TGME49_{—}000470_{—}1, TGME49_{—}001390_{—}1, TGME49_{—}004130_{—}13, TGME49_{—}005300_{—}6, TGME49_{—}005360_{—}14, TGME49_{—}005740_{—}9, TGME49_{—}012300_{—}5, TGME49_{—}013340_{—}4, TGME49_{—}014610_{—}11, TGME49_{—}014760_{—}2, TGME49_{—}016180_{—}1, TGME49_{—}016380_{—}13, TGME49_{—}016380_{—}5, TGME49_{—}021310_{—}9, TGME49_{—}023540_{—}10, TGME49_{—}023540_{—}5, TGME49_{—}024190_{—}10, TGME49_{—}024920_{—}4, TGME49_{—}025320_{—}5, TGME49_{—}026020_{—}8, TGME49_{—}026110_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044040_{—}8, TGME49_{—}044080_{—}3, TGME49_{—}044280_{—}1, TGME49_{—}045500_{—}3, TGME49_{—}046330_{—}5, TGME49_{—}046340_{—}2, TGME49_{—}047370_{—}4, TGME49_{—}047370_{—}9, TGME49_{—}048200_{—}3, TGME49_{—}048670_{—}5, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057080_{—}9, TGME49_{—}057520_{—}1, TGME49_{—}058390_{—}1, TGME49_{—}058980_{—}1, TGME49_{—}059200_{—}2, TGME49_{—}061740_{—}1, TGME49_{—}062920_{—}5, TGME49_{—}063560_{—}6, TGME49_{—}064740_{—}4, TGME49_{—}066760_{—}1, TGME49_{—}067350_{—}1, TGME49_{—}068590_{—}4, TGME49_{—}068590_{—}9, TGME49_{—}070220_{—}3, TGME49_{—}070250_{—}1, TGME49_{—}070250_{—}2, TGME49_{—}072290_{—}1, TGME49_{—}073380_{—}3, TGME49_{—}074060_{—}5, TGME49_{—}074190_{—}2, TGME49_{—}078660_{—}9, TGME49_{—}085240_{—}1, TGME49_{—}085240_{—}3, TGME49_{—}086120_{—}1, TGME49_{—}086450_{—}1, TGME49_{—}088400_{—}9, TGME49_{—}088500_{—}5, TGME49_{—}089380_{—}3, TGME49_{—}089730_{—}4, TGME49_{—}090580_{—}5, TGME49_{—}090870_{—}5, TGME49_{—}090950_{—}5, TGME49_{—}092220_{—}1, TGME49_{—}095650_{—}4, TGME49_{—}097240_{—}6, TGME49_{—}099060_{—}4, TGME49_{—}099060_{—}6, TGME49_{—}100060_{—}2, TGME49_{—}100310_{—}7, TGME49_{—}101270_{—}10, TGME49_{—}105020_{—}9, TGME49_{—}105270_{—}2, TGME49_{—}105510_{—}3, TGME49_{—}105510_{—}5, TGME49_{—}109910_{—}2, TGME49_{—}112600_{—}3, TGME49_{—}113020_{—}8, TGME49_{—}114850_{—}4, TGME49_{—}118460_{—}3, TGME49_{—}16180_{—}1, TGME49_PP2C-hn, TGME49_TLN_{—}1, and fragments thereof.

2. The antigen composition of claim 1 wherein the known reactivity is characterized by strength of immunogenicity.

3. The antigen composition of claim 1 wherein the known reactivity is characterized by activity state of the disease.

4. The antigen composition of claim 1 wherein the parameter is selected from the group consisting of a previous exposure to the pathogen, duration of exposure to the pathogen, acute infection, chronic infection, no infection, at least partial immunity to infection with the pathogen, and expected outcome upon treatment.

5. The antigen composition of claim 1 wherein the antigen is present in at least 40% of a population exposed to said antigen, and optionally wherein at least one of an average binding affinity and an average quantity of antibodies produced in a patient against the antigen is in an upper tertile of binding affinity and quantity of antibodies produced in the patient.

6. The antigen composition of claim 1 wherein the disease parameter is previous infection with Toxoplasma gondii, and wherein the antigen is selected from the group consisting of: TGME49_{—}000470_{—}1, TGME49_{—}001390_{—}1, TGME49_{—}004130_{—}13, TGME49_{—}005300_{—}6, TGME49_{—}005360_{—}14, TGME49_{—}005740_{—}9, TGME49_{—}012300_{—}5, TGME49_{—}013340_{—}4, TGME49_{—}014610_{—}11, TGME49_{—}014760_{—}2, TGME49_{—}016180_{—}1, TGME49_{—}016380_{—}13, TGME49_{—}016380_{—}5, TGME49_{—}021310_{—}9, TGME49_{—}023540_{—}10, TGME49_{—}023540_{—}5, TGME49_{—}024190_{—}10, TGME49_{—}024920_{—}4, TGME49_{—}025320_{—}5, TGME49_{—}026020_{—}8, TGME49_{—}026110_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044040_{—}8, TGME49_{—}044080_{—}3, TGME49_{—}044280_{—}1, TGME49_{—}045500_{—}3, TGME49_{—}046330_{—}5, TGME49_{—}046340_{—}2, TGME49_{—}047370_{—}4, TGME49_{—}047370_{—}9, TGME49_{—}048200_{—}3, TGME49_{—}048670_{—}5, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057080_{—}9, TGME49_{—}057520_{—}1, TGME49_{—}058390_{—}1, TGME49_{—}058980_{—}1, TGME49_{—}059200_{—}2, TGME49_{—}061740_{—}1, TGME49_{—}062920_{—}5, TGME49_{—}063560_{—}6, TGME49_{—}064740_{—}4, TGME49_{—}066760_{—}1, TGME49_{—}067350_{—}1, TGME49_{—}068590_{—}4, TGME49_{—}068590_{—}9, TGME49_{—}070220_{—}3, TGME49_{—}070250_{—}1, TGME49_{—}070250_{—}2, TGME49_{—}072290_{—}1, TGME49_{—}073380_{—}3, TGME49_{—}074060_{—}5, TGME49_{—}074190_{—}2, TGME49_{—}078660_{—}9, TGME49_{—}085240_{—}1, TGME49_{—}085240_{—}3, TGME49_{—}086120_{—}1, TGME49_{—}086450_{—}1, TGME49_{—}088400_{—}9, TGME49_{—}088500_{—}5, TGME49_{—}089380_{—}3, TGME49_{—}089730_{—}4, TGME49_{—}090580_{—}5, TGME49_{—}090870_{—}5, TGME49_{—}090950_{—}5, TGME49_{—}092220_{—}1, TGME49_{—}095650_{—}4, TGME49_{—}097240_{—}6, TGME49_{—}099060_{—}4, TGME49_{—}099060_{—}6, TGME49_{—}100060_{—}2, TGME49_{—}100310_{—}7, TGME49_{—}101270_{—}10, TGME49_{—}105020_{—}9, TGME49_{—}105270_{—}2, TGME49_{—}105510_{—}3, TGME49_{—}105510_{—}5, TGME49_{—}109910_{—}2, TGME49_{—}112600_{—}3, TGME49_{—}113020_{—}8, TGME49_{—}114850_{—}4, TGME49_{—}118460_{—}3, TGME49_{—}16180_{—}1, TGME49_PP2C-hn, TGME49_TLN_{—}1, and fragments thereof.

7. The antigen composition of claim 1 wherein the disease parameter is acute infection with Toxoplasmos gondii, and wherein the antigen is selected from the group consisting of: TGME49_{—}001390_{—}1, TGME49_{—}004130_{—}13, TGME49_{—}012300_{—}5, TGME49_{—}014760_{—}2, TGME49_{—}016380_{—}13, TGME49_{—}023540_{—}5, TGME49_{—}024190_{—}10, TGME49_{—}024920_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}037150_{—}5, TGME49_{—}040870_{—}16, TGME49_{—}042790_{—}18, GME49_{—}044080_{—}3, TGME49_{—}044280_{—}1, TGME49_{—}045500_{—}3, TGME49_{—}046330_{—}5, TGME49_{—}047370_{—}9, TGME49_{—}048840_{—}2, TGME49_{—}048840_{—}3, TGME49_{—}054570_{—}6, TGME49_{—}057080_{—}5, TGME49_{—}061740_{—}1, TGME49_{—}064740_{—}4, TGME49_{—}068590_{—}9, TGME49_{—}070220_{—}3, TGME49_{—}078660_{—}9, TGME49_{—}086120_{—}1, TGME49_{—}086450_{—}1, GME49_{—}088500_{—}5, TGME49_{—}089380_{—}3, TGME49_{—}092220_{—}1, TGME49_{—}095650_{—}4, TGME49_{—}097240_{—}6, TGME49_{—}099060_{—}4, TGME49_, TGME49_{—}100060_{—}2, TGME49_{—}100310_{—}7, TGME49_{—}101270_{—}10, TGME49_{—}105510_{—}5, TGME49_{—}112600_{—}3, TGME49_{—}16180_{—}1, TGME49_TLN_{—}1, and fragments thereof.

8. The antigen composition of claim 1 wherein the disease parameter is chronic infection with Toxoplasma gondii, and wherein the antigen is selected from the group consisting of: TGME49_{—}001390_{—}1, TGME49_{—}014610_{—}11, TGME49_{—}027620_{—}2, TGME49_{—}057520_{—}1, TGME49_{—}058980_{—}1, TGME49_{—}070250_{—}2, TGME49_{—}086450_{—}1, and fragments thereof.

9. The antigen composition of claim 1 wherein the disease is toxoplasmosis, and wherein the antigen is selected from the group consisting of: TGME49_{—}000470_{—}1, TGME49_{—}001390_{—}1, TGME49_{—}004130_{—}13, TGME49_{—}005300_{—}6, TGME49_{—}005360_{—}14, TGME49_{—}005740_{—}9, TGME49_{—}012300_{—}5, TGME49_{—}013340_{—}4, TGME49_{—}014610_{—}11, TGME49_{—}014760_{—}2, TGME49_{—}016180_{—}1, TGME49_{—}016380_{—}13, TGME49_{—}016380_{—}5, TGME49_{—}021310_{—}9, TGME49_{—}023540_{—}10, TGME49_{—}023540_{—}5, TGME49_{—}024190_{—}10, TGME49_{—}024920_{—}4, TGME49_{—}025320_{—}5, TGME49_{—}026020_{—}8, TGME49_{—}026110_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044040_{—}8, TGME49_{—}044080_{—}3, TGME49_{—}044280_{—}1, TGME49_{—}045500_{—}3, TGME49_{—}046330_{—}5, TGME49_{—}046340_{—}2, TGME49_{—}047370_{—}4, TGME49_{—}047370_{—}9, TGME49_{—}048200_{—}3, TGME49_{—}048670_{—}5, TGME49_{—}048840_{—}2, TGME49_{—}048840_{—}3, TGME49_{—}054370_{—}11, TGME49_{—}054570_{—}6, TGME49_{—}057080_{—}5, TGME49_{—}057080_{—}9, TGME49_{—}057520_{—}1, TGME49_{—}058390_{—}1, TGME49_{—}058980_{—}1, TGME49_{—}059200_{—}2, TGME49_{—}061740_{—}1, TGME49_{—}062920_{—}5, TGME49_{—}063560_{—}6, TGME49_{—}064740_{—}4, TGME49_{—}066760_{—}1, TGME49_{—}067350_{—}1, TGME49_{—}068590_{—}4, TGME49_{—}068590_{—}9, TGME49_{—}070220_{—}3, TGME49_{—}070250_{—}1, TGME49_{—}070250_{—}2, TGME49_{—}072290_{—}1, TGME49_{—}073380_{—}3, TGME49_{—}074060_{—}5, TGME49_{—}074190_{—}2, TGME49_{—}078660_{—}9, TGME49_{—}085240_{—}1, TGME49_{—}085240_{—}3, TGME49_{—}086120_{—}1, TGME49_{—}086450_{—}1, TGME49_{—}088400_{—}9, TGME49_{—}088500_{—}5, TGME49_{—}089380_{—}3, TGME49_{—}089730_{—}4, TGME49_{—}090580_{—}5, TGME49_{—}090870_{—}5, TGME49_{—}090950_{—}5, TGME49_{—}092220_{—}1, TGME49_{—}095650_{—}4, TGME49_{—}097240_{—}6, TGME49_{—}099060_{—}4, TGME49_{—}099060_{—}6, TGME49_{—}100060_{—}2, TGME49_{—}100310_{—}7, TGME49_{—}101270_{—}10, TGME49_{—}105020_{—}9, TGME49_{—}105270_{—}2, TGME49_{—}105510_{—}3, TGME49_{—}105510_{—}5, TGME49_{—}109910_{—}2, TGME49_{—}112600_{—}3, TGME49_{—}113020_{—}8, TGME49_{—}114850_{—}4, TGME49_{—}118460_{—}3, TGME49_{—}16180_{—}1, TGME49_PP2C-hn, TGME49_TLN_{—}1, and fragments thereof.

10. The antigen composition of claim 1 wherein the disease is early-stage toxoplasmosis, and wherein the antigen is selected from the group consisting of: TGME49_{—}001390_{—}1, TGME49_{—}004130_{—}13, TGME49_{—}012300_{—}5, TGME49_{—}014760_{—}2, TGME49_{—}016380_{—}13, TGME49_{—}023540_{—}5, TGME49_{—}024190_{—}10, TGME49_{—}024920_{—}4, TGME49_{—}026730_{—}9, TGME49_{—}027620_{—}2, TGME49_{—}031430_{—}2, TGME49_{—}033710_{—}4, TGME49_{—}034410_{—}17, TGME49_{—}037150_{—}5, TGME49_{—}040870_{—}16, TGME49_{—}042790_{—}18, GME49_{—}044080_{—}3, TGME49_{—}044280_{—}1, TGME49_{—}045500_{—}3, TGME49_{—}046330_{—}5, TGME49_{—}047370_{—}9, TGME49_{—}048840_{—}2, TGME49_{—}048840_{—}3, TGME49_{—}054570_{—}6, TGME49_{—}057080_{—}5, TGME49, TGME49_{—}061740_{—}1, TGME49_{—}064740_{—}4, TGME49_{—}068590_{—}9, TGME49_{—}070220_{—}3, TGME49_{—}078660_{—}9, TGME49_{—}086120_{—}1, TGME49_{—}086450_{—}1, GME49_{—}088500_{—}5, TGME49_{—}089380_{—}3, TGME49_{—}092220_{—}1, TGME49_{—}095650_{—}4, TGME49_{—}097240_{—}6, TGME49_{—}099060_{—}4, TGME49_, TGME49_{—}100060_{—}2, TGME49_{—}100310_{—}7, TGME49_{—}101270_{—}10, TGME49_{—}105510_{—}5, TGME49_{—}112600_{—}3, TGME49_{—}16180_{—}1, TGME49_TLN_{—}1, and fragments thereof.

11. The antigen composition of claim 1 wherein the disease is chronic toxoplasmosis with persistent IgM, and wherein the antigen is selected from the group consisting of: TGME49_{—}001390_{—}1, TGME49_{—}004130_{—}13, TGME49_{—}005300_{—}6, TGME49_{—}005350_{—}14, TGME49_{—}014760_{—}2, TGME49_{—}016380_{—}13, TGME49_{—}024190_{—}10, TGME49_{—}025320_{—}5, TGME49_{—}026020_{—}8, TGME49_{—}031430_{—}2, TGME49_{—}033710_{—}4, TGME49_{—}035020_{—}9, TGME49_{—}035160_{—}2, TGME49_{—}035660_{—}1, TGME49_{—}037150_{—}5, TGME49_{—}040870_{—}16, TGME49_{—}042790_{—}18, TGME49_{—}043580_{—}1, TGME49_{—}044080_{—}3, TGME49_{—}047370_{—}9, TGME49_{—}048200_{—}3, TGME49_{—}048840_{—}2, TGME49_{—}048840_{—}3, TGME49_{—}054370_{—}11, TGME49_{—}057080_{—}5, TGME49_{—}057080_{—}9, TGME49_{—}059200_{—}2, TGME49_{—}061740_{—}1, TGME49_{—}062920_{—}5, TGME49_{—}064740_{—}4, TGME49_{—}066760_{—}1, TGME49_{—}067350_{—}1, TGME49_{—}068590_{—}9, TGME49_{—}070250_{—}1, TGME49_{—}070250_{—}2, TGME49_{—}073380_{—}3, TGME49_{—}074190_{—}2, TGME49_{—}085240_{—}1, TGME49_{—}085240_{—}3, TGME49_{—}086450_{—}1, TGME49_{—}089730_{—}4, TGME49_{—}090580_{—}5, TGME49_{—}090870_{—}5, TGME49_{—}090950_{—}5, TGME49_{—}099060_{—}4, TGME49_{—}100060_{—}2, TGME49_{—}100310_{—}7, TGME49_{—}105270_{—}2, TGME49_{—}105510_{—}3, TGME49_{—}105510_{—}5, TGME49_{—}112600_{—}3, TGME49_{—}113020_{—}8, TGME49_{—}114850_{—}4, TGME49_PP2C-hn, TGME49_TLN_{—}1, and fragments thereof.

12. The antigen composition of claim 1 wherein the disease is chronic toxoplasmosis, and wherein the antigen is selected from the group consisting of: TGME49_{—}001390_{—}1, TGME49_{—}014610_{—}11, TGME49_{—}027620_{—}2, TGME49_{—}057520_{—}1, TGME49_{—}058980_{—}1, TGME49_{—}070250_{—}2, TGME49_{—}086450_{—}1, and fragments thereof.

13. The antigen composition of claim 1 wherein the carrier is a pharmaceutically acceptable carrier, and wherein the composition is formulated as a vaccine.

14. The antigen composition of claim 13 wherein the vaccine comprises at least four antigens.

15. The antigen composition of claim 13 wherein the antigens or fragments thereof are recombinant.

16. The antigen composition of claim 13 wherein the antigens or fragments thereof are at least partially purified.

17. The antigen composition of claim 1 wherein the carrier is an insoluble carrier.

18. The antigen composition of claim 17 wherein each of the antigens are present in a purity of greater than 60%.

19. The antigen composition of claim 17 wherein the antigens or fragments thereof are recombinant.

20. The antigen composition of claim 17 wherein the antigens or fragments thereof are at least partially purified.

21. The antigen composition of claim 17 wherein the insoluble carrier is a testing dipstick.

22. The antigen composition of claim 17 wherein the insoluble carrier is a testing array.

23. The antigen composition of claim 22 wherein the testing array is an essentially planar array.

24. The antigen composition of claim 22 wherein the testing array is a fluid suspension array.


SEQUENCE LISTING (100 sequences)

SEQ ID NO: 1 - DNA, 336 bp, Toxoplasma gondii
atgcgtgcctccaagtcgtctcctgggtgctcgtcttcagttttttctctctcatccttcgcacagagcaccgtccgcctcaccacgcagtctcgaggctgtcatctggcgggaggtgcagtagagaatgaagttgagaagctgcgtcagagcctagctgataccttggcccgtcacgtgccagagaatcgtggaaaacgtcgcggagacggcctccgaggagacagctggattgctcacatcgcggttgtgtcccctggtgtagcgatttcagtcaacgaaaattgcgaccccactgtgcgcggtgacatggtcacagccctcaatttgctttcg

SEQ ID NO: 2 - DNA, 327 bp, Toxoplasma gondii
atgaagactcttcatttacttcttttcgcggtggtggccatcgcgctttgtggcctggccacagcgagccacggcacaacgtttcaagacgcaggtgcaagagcggatgcctttgcagccagggtggcgggtgcgagggcggcggtagtttccggcttgacaagcgagaacggcctttggcgtcgcactcaagcaaggttaggcttctggcccggttacctcgtgggtgccgctgcagttgccgtggccttggtgacagggtatgcactggggaagaggagagagaggtctaaatggaagaaggccctgaagaatcggctggactaa

SEQ ID NO: 3 - DNA, 402 bp, Toxoplasma gondii
tatgttatggaagcgcacacgaacctgcagtttgtacgcgacaaattcctacaatgtcccgaaaacgccagtgagtgcaagatgaccggaaaaggtgtggaccatggaatgctgtggctgtttgatcggcacgccctctttggctggattatctgcaaaacggtgaacgagccggctatgcatgtagcaacggacgtgggcaaggcgaaggggaacggcaaaaagagaaagggaaagaagggtaagaataagactaatgcgcctaacgaagtcgaggaagggcagcagctgggcgcggattcaccgagccaagtctctgtaccagccgatgctgattcagggccgacgtccaagactatgtcgagtctcaagcttgcgcccgtcaagctgttagacctgtaa

SEQ ID NO: 4 - DNA, 225 bp, Toxoplasma gondii
tcgtactgcggatctgcctactggggcgtgtacgtccttggcgcggccttcctgatgagttgctcgtacgcagcggcgatgtggctctggagaaacaacgaaaaagaacgggggaaaattgaggtcgaggggacaaccagagcacaacccaagaagggggagcttgagtactcttttgcgcatgtccacagtctcatgggcatttcgttggtcgccggcatgatg

SEQ ID NO: 5 - DNA, 243 bp, Toxoplasma gondii
cccatcaacgcgatgggtggaagacggtttctgcacgtcatgttcgtcgcgtttgtggagttgtttccaaaccccgatgatttccctcgtcaggtgcttctcacccccatgcaaatgctccagttttgtggactgcagactgtgttgaggacctacacagctccctacttttttgacagaaagaaagggaacagtttggcgctgcaaatgaaggcttctcctgttccctccgagtcacacaga

SEQ ID NO: 6 - DNA, 252 bp, Toxoplasma gondii
acacgcctggtgtggctggacgcaactgtcggcacggtgttgcacgtggcggtggtgccattcgtgccagcgcacatcgtcccgctccaagctgttagtccctatcatcaccaggtggcaagatccacaactgagcagaacactcaaaccttcttcgagagaacaaacggggcccttgtggtcgacgccagcggcaaggcgtcggttcttatccccaggaaggcagagaccgaaactgagctcgcgacgctg

SEQ ID NO: 7 - DNA, 363 bp, Toxoplasma gondii
ttgccgatcttcgtcgtgacgcagtgcccggactccatggagagagtggcgttcatgaatacctacctggtgccatcgaaacgcaactttcgcgccgtgtttcaagctttgggacatacacctcaagcctctgctgttgtcatccggattgagtcagttatggttgaggcttggatggaggtgaatggaaaagtgacaggcatgcagtacctcacgcctcctgctcacgaggcgttgaccgtgaccaccaaggatcggttcggcttcgaatttctcaggcgcgtgagagagcttgaaacaggggattttaaggctgatccacacggacgcagcgtcgttattgcgtttgtggggaatcggaag

SEQ ID NO: 8 - DNA, 378 bp, Toxoplasma gondii
ggatggaaggcacaggcatactatttgatgaaacgtgcgaaagctgcgctgatgtattacgccaagaacgaccctgaagagtcaaagattctgaagcgagcatgtgtgggagtagacggagagatcacagtgtccgcgggtgcaatacacaccccgctgttgctcatacgatcgggaatcggttcccgtgaacaactgcgagagattaacgtccctactgtgaaagagttgccagctgtgggaaaaaacttgagggatcggatgtttattcccctgaatttctttgctgtcgggcaagacaccagtccgcggcgtcacccaacaagaatatgcgaggcgactgggatcgcgaaactcggacctgactgcgaaaacttc

SEQ ID NO: 9 - DNA, 396 bp, Toxoplasma gondii
ctttctctggaactctttggcttcatccgcctcctgcagcccttctgtctccacgggggcctgcgcccagaggcgcgcttcaacctccggcactcgcacctgccctcggacgagctctcacattcatctcccgatgtgctacactcctccgatggggactcgcaagcgaaactcttcggtgaaggcggaagctcgggggccgctgccgcctcccgctgcgcttctcaaagggatggacggcgctccagcttccagtgtttaggcagctggctacagttcggaggggcgccgagtgacttgttcgacgcagacgggaatccccgtccagatgttccgatcgactttctcctgcaagctgagaactataagctcctccccacaggagtccccggacaa

SEQ ID NO: 10 - DNA, 390 bp, Toxoplasma gondii
gcgactgcggtgttcgtgcttgataatgacggcagctgtttgcctgcgctctacgcacatgcgaagcgtcttcgctggctgcacagtatgtctgctggcgtcgactgcttttcgtttttcctcaactccacaccgtcgcacaagagcgcctctcaaacgctcgccgaagatcccggattcctcgtcaccaacggcaagggcgttttctcgcgacccctggcagagtatgtcgccgctgcgatgctgcactttgccaaggacatttgtcgcctgcagaaactgaaacacgagcgctcgtggaaccctgcgcccatgcaccagttgcggggaaagactgtaggctttctcggttacggcgacattgcaagagaaacagcgaaactcctccgg

SEQ ID NO: 11 - DNA, 444 bp, Toxoplasma gondii
atgataactgttattttgtgttcggtgctcttctattcgttgggccttgtggccccaacctcggcggcattgcatgcttgcattaagcagaaaacattgcccaacgaaaacgcgatgctgctgtggacacagtacttcctgcttttttctgctctcgtcactgttgtgtttccatacgttgtgactcctcttttcttcttgctgccgtcctggctacttgctatggtgaaactggttctagtggtggcacttgctgttcccaagctcggcttgacctcacgattttacgggtggtttctgtgccattatgtcgaatacctcgatttgattgctaaagctcttcagcagcatgtggtgatccctcttaagacttacgttagtgatgctattgcccgcatgcaagccagcaccgaggcctccgtatctactcggaaggagctgtag

SEQ ID NO: 12 - DNA, 219 bp, Toxoplasma gondii
tgctcggattttggagtcacgcagtttcgactgctcctgcctctcctgctcgtgcatggccggtggtgctaccggcgaataacgaccgtcattctttacatcatctacaagaactttcttctcgtcttgcccctagtctacttcggatttctctgcctcttctctgggcagcggttctaccctgaagtcctcgcccaaacctacaatccagttcttact

SEQ ID NO: 13 - DNA, 204 bp, Toxoplasma gondii
aattctgggccgcctcgatcgaagcgatctcgcgtcgagagactctcaggtcgaatgattgtgggaatcgccgtgcttcaagccatcatgtgcactgccacagccatcgccagcactgttctgtggtattcgccttcgtctccgttgaagaatttgacttatctgcacaatccgcaggaccggagtctccctgggtttctcttc

SEQ ID NO: 14 - DNA, 225 bp, Toxoplasma gondii
gctttgcttctgacgcttccgagttattcgcgtctcgagcaagacgcacctcggcctcttgatcctgacgctattatctctgcacgccgttcgcttctccgggatatttactacttccatcggaacgccctggatgaagcttacgtagcgaccacgatccccaaagttgacgatagagaacgggacaggcagctcgaatcggcagaagatcctgaacaatggcaa

SEQ ID NO: 15 - DNA, 351 bp, Toxoplasma gondii
tacgagcagaagaaagtctttattttggggtgctcgcggcttgttcaggagtttgcggcagcgccggcgtcgttcccagcatgcgttgcagagcgagtagaggttatctttaaagtgctcgccacgcaagtcactgagcaggcacagctgaagcagaagttgaaggaggaggaagagaacagcgacttcgacacagattcggatggagactccgagcaagacctcgacgaaacggaagacgcagggctgcacccgaaagccaaagaaatcctcgagaaactcgacaacgccaagtggggtgacgacgaggacgacgacgacgatgactgggacgaaatgtgtgactcgagc

SEQ ID NO: 16 - DNA, 351 bp, Toxoplasma gondii
tggccgctgctgctgcctgcagtgcggagcgacatcgcggcgcgccaagactcgagtcgcctcatgtgcgcgctgtctgtcctgcggagactctgcggaatttatgaattcaaacgcaccgacaaagaggctctggactcgatcattgaacagacctggccgctgctgctgccggctgcggcacagctgctgaacgagggcggcttgagcaactcggacgccatgcagatgctcaagctgatttgtaaagtctactggagttcgactcaagtctgtctcgcctcctccggcctcgttgtctccaccatggacgactggatggagctcatggaacagattctcgtccgaccc

SEQ ID NO: 17 - DNA, 369 bp, Toxoplasma gondii
gtgaggacctcgacgctgccggaagagttagggaggatcgacttcctcttcagcgacaagacagggacgctgactcggaacgagatgcacttcaagcgcctgcacatcggccgcgcgatgttcgcggaagatggcctcaccgaactccgcatcttcctcgagagctacttccttcgctcgcatcctgtctcgcctgcgaacgccgaccagcccagttggagagacagagagcgagcaagaacgggagagagagaacgcggacgagaactgggtgggcgacgcggagaagttgtgcatgcagcagtcgaggcgattcgtgctctcagtctctgccacaacgtcacccccgtgaaagacgaggacagtcag

SEQ ID NO: 18 - DNA, 459 bp, Toxoplasma gondii
tgttgcccatcaactgacctcaactgcaaaggacccgccgaaggttctgctgcttcttccacgtcttcctcccccgtttcctctgtctccggcgtttcgcctgtgcactggtcgtttgtggtcgggcataccctgggcgtcgaccatgtaggacacaaagctgaagttcgcagtccgctgctgagcgccaagctcaggcagatggatcgcctcattctgaatttggtgatacatctgctcgagcagtctgcagacgagtgtggctctgcaaaggcgcgtaaacagcaaggcacacctgtctcagtccctgcttccccgtcttcttctccgtcttcgtcatctagtggaggttccttggatgctgcctcgacacctgccgtcaaccgaactcttctgatggtcatgggagaccacggcatgactgatgatggagcccatggaggcgcgctgaacgaggaa

SEQ ID NO: 19 - DNA, 462 bp, Toxoplasma gondii
tggctgcccatcccaaatgatctgacatttccagtaaccaccaaaaactttgttattcggatgcagtgcagtggcgtccagtcatgcaacatgaacaggatagcagtgtgcgcacgtatcacgtgtccagccacggcaacagtggctgaacagcagatggctgctgtgcaagacgctatgagcaaacctgttggcaacggcccagccagtcgcgccgctggtcttgcttccggggatagcccagcttctcgagtgatgcaggcatcgactgggcggtttggttccacgttaagctcgcaggtaccacttgttcatcccggggcatctccgtattcccagcaccatggcgcttttccacacggatacccctaccttgtcctcggccacgcggcaactcgtgttccatccgccttcgtatcagcattcttttgctccgtcgcctcaatcgctcttttcttttaa

SEQ ID NO: 20 - DNA, 243 bp, Toxoplasma gondii
cacgaagcgcgatttgccggtgtgttcatgtttttcgcgaacatcatggctggagttgctctcgaagtaatgctgttgctgctcgaacacttcggatacgatggcacaaagaaaatgggagaagctgaaccagaggctgtgatcatgacgatcagagttggctacggcgctgcactggcgttgatgctcattttcatcaccccgtcgatgattctgtatccaatcacgaggaaaagtcatgag

SEQ ID NO: 21 - DNA, 438 bp, Toxoplasma gondii
gggtgcaaaacgcgctatcctacgatggactgtcttgtcgctctgagcacgaatcttgcctttttctactcgtgctttgccctcttgtcgagttttttatcccggctggttttcgcaagccacatggacgactttaagaggcatttctcgaccgatgctgcaaccggaacaaaggatttttgggcgtccacgcaaaacgacttcttcgtgcatgcagcttcggaagcggaaaacgacccccccacgtgcttcgacgcatgcgcaacgctcaccacggtgttgcttattggaaaggtacttcatgcctacagagagaaggacgagggagatagcgtggaccaagacgttcttgctgctcttgcgcttctcattaagggcagtcggggcagacgctcgtcgctctcttttgcctgtcagcgtggaacatttcaactggtg

SEQ ID NO: 22 - DNA, 336 bp, Toxoplasma gondii
ggcccgtacttgatgggtgtcaaggcgatcattgcggagtctttcgagcgcattcaccgcaccaacttggtcggaatgggcatcttgcctctccagttccaggaaggacagaacgccgagtcgttgggcttgacaggcaaggagcagttcaacatttcgcttaacaagggagaaatcatcccaggatctctcatgactgtaaagacaagcgacggaaaggccttcgacgtacgatgcagaatcgacaccgaactcgaagtgaagtacttccagaacggcggcattctccactatgtgttgaggaatcttacgaagcagcactccggggaaaactga

SEQ ID NO: 23 - DNA, 408 bp, Toxoplasma gondii
ggaaaaggtgaacatacaccaccactcccagacgagaggcaacaagagccagaagaaccggtttcccaacgtgcatccagagtggcagaacaactgtttcgcaagttcttgaagttcgctgaaaacgtcggacagcacagtgagaaggccttcaaaaaagcaaaggtggtggcagaaaaaggcttcaccgcggcaaaaacgcacacggttaggggtttcaaggtggccaaagaagcagctggaaggggcatggtgaccgttggcaagaaactcgcgaatgtggagagtgacagaagcactacgacaacgcaggcccccgacagccctaatggcctggcagaaacccaggctccagtggagccccaacagcgggccgcacacgtgcccgtcccagacttttcgcagtaa

SEQ ID NO: 24 - DNA, 204 bp, Toxoplasma gondii
atccaaacggtgggcagacggtggagccccgtcgtggaggtggctgctcttcttgtcattttcgttctgtgtttctgcattcggcaatttgccgtcattcgttatgagtccgtcatccacgaatttgacccctacttcaattaccgaacaacgacatatctcgccaaggaagggttctacgagttctggaactggtttgaccac

SEQ ID NO: 25 - DNA, 333 bp, Toxoplasma gondii
gcagcgacgattgcggcgtatgtgacgcgcaggtgtgcagagtctgcatgcacgtacgagggcgtggagacgctggtggcgtcggtgtttctggcaggactgattttgatgatcttcgcgtacagccgcctggcatgtctcgtccagctgattcctgcgtccgtcacgattgggttctgtaacggcatcgccatcatcatcttcctggcgcaacttcagattttcaaagatccctcaacaggcgcctacatcacgggcaaccgcgcagcctggatggctggcgaatgttgtctagccgcagtcatcatggagggctggaaaaaggttcctttt

SEQ ID NO: 26 - DNA, 249 bp, Toxoplasma gondii
gtgtctctgtggatgacgtgcgtcgaaggctggggaaacgtcctgaaggtcttgcggacgcgcacagaagtctacttttatcttttgaaacaactcgcgcggctccacatttccttccaactgcctctccagcccatccacttcccttcgacctccggggcctcccttcagactgcaggcagtggaaacccagcagccggcaggctcacgcctgcttctccgcaagaaagaaaagtcgtcagtgccgca

SEQ ID NO: 27 - DNA, 225 bp, Toxoplasma gondii
gctttgcgaaacaagacgttcgggaagtgcgttgactttgtgtggtcggctgacggtcactatgcgattcgagaggaaagcaatcgcatccgcgttcacacaagcttcacagaaaccttcaactttactccccccttctcagtagagacgctgtggggtggagcccttctcgctctcaaggccagcgacgactctttcgtctgcttctacgattgggaagcttgc

SEQ ID NO: 28 - DNA, 207 bp, Toxoplasma gondii
atcgcccaatggggacgccgacagctggcgtatgaggccagcgtaaaggcgaactcgggctccggagacactctgcgaaatgatgagctacgagcaccgctcctagagttcgagtcgaaagttgtcttccttattcagggaatgctgcggctgcttcaggctcatgcagtcctccaggacaccaactcagtccaggcgacagactac

SEQ ID NO: 29 - DNA, 201 bp, Toxoplasma gondii
atggcgactaaccaccgtctttgttcctcctttcttctccttgctctgcttttttccgtctctctccagcagttctcaagcctctcctctctctctgcttcttctgccgccgcgtctctttctcccttatctctctgcctcggcgcacaaggcgcgagtaccgaggagctgacgtcgaactctgcggacgaggaagacgac

SEQ ID NO: 30 - DNA, 204 bp, Toxoplasma gondii
tctttcgagggtccggcgaagctgattttccctgtcagtatccacccgtggcagcacagcattctcggcttgggagacattgtcattccgggagtcttcatctccatgtgtctccgcttcgactactcgctggccacggcttccgtcacaaacggaaacgcggcgaaaacgaccactgtcggcgcgtcgatcgacatccaccag

SEQ ID NO: 31 - DNA, 213 bp, Toxoplasma gondii
cgcattcagctgcatttgacacttgcgaacaagagctcgatgactctcaacggctgggcgattcagttcaacagaaactccttcggccttgctccagccgcaaacctgcaagtcgccgacttgctctctggccagtctgcagagacgactgtgccagtggttccagggcaactcatgtccaacgctgcccctgagcagcctctctctctgcaa

SEQ ID NO: 32 - DNA, 225 bp, Toxoplasma gondii
ggatggatggtgtgtattcgcggtcgactgaaggtcgttcgcactcctctaacctgcgcagatgtttgggtcgacttccgcgtggagttgaacgtttttcgcctttccagaatcatttctgcacagagcgcactggtcaccgaactggcagacagagagagggaactcgccgacgtcaagcgacagctggggaagctcgaagcgagtgacgcgacgcgcgctgag

SEQ ID NO: 33 - DNA, 468 bp, Toxoplasma gondii
atgcttccgacgcggtgtttgaagtttgtaactctctctactgccatgttgtccctccaaactccaagactttctgcatttttcagctgcccacttccttttcgccgtttcaatttacccttttccggtccggcggcctctgaccgtctctcgggtgtctcgtctctgcggacgcaggaacacgcacgtcgacgcgaacgcgcgctctgcgcttttccttgctcgcactcaaaaccgcaagcttcggtcaaccggggtttttcagcgctgaatttcgatattcctacttctttcctgctcactcacggcgcgtgcggctctagacagagtacaagaaacgttcctcagcggcgctaccagaccaagagaaacatggcgaccaaggcgcaggatggccataagatcgtggcctacgatccagaaaatgtgttcaaaaagattcttgacggcaaaatcccctgccataag

SEQ ID NO: 34 - DNA, 474 bp, Toxoplasma gondii
gaagtcaccgttttgacatccgtcgcgcgaatcgtcctcaggtgtctgcgcagccgcgagtcgttctcgcgtctcacagctctcgtatggctgcatgcgatcctctccctcgctctccaccctgcacctgccgctgccgcctcgtcctcgccgaccgcggagactctcagcgacaacaggcaggaggtggagagacgcctgaaaaacgaaaagcgaaggaacaaagaaaccgcaaacgagaaaggcagcggccgcacgcagagcggacgagaaggcatgcaacaagaagcggcagacagagacgaccccgaagagaaggaacgtgaagaagatggaagtggagaagatggcagcgcgatagcagaaaagacagaagataatgtagaagaaaatgatgtcgaaggcggagctgacgaagaagacgagaatcataaacgtcacctgtggcagatggctcttcagagagaactggag

SEQ ID NO: 35 - DNA, 339 bp, Toxoplasma gondii
ccatcattcctttcgtttcctcctgaactgcaggcgctgtaccgttggcgcatcacccccgcgaaacgttctcgcgtaaaaggctggacggacgaactcgtttgtgtggtggagaaaacgcaagtggctgaccagatgcaaatgcaagtgaatcagagtgcctccgaggcagctgtccgagcctccatgcccgctgctggattcgctgcaggcccttctccctttgtcaatccctatgccttcagtcaggctttaccttcggtgcatgcaggcactgcgagtctcttttccgtttccctcgcgttcctcgcttctgcactgactctcctaaagtcttag

SEQ ID NO: 36 - DNA, 390 bp, Toxoplasma gondii
atggagatatgtcaactgcctcgtgtgcgtcacctcaggatgatggtggctgtcgcgttaggtgccctcctcttcctctcgatctgtggggaacggatcctcgggagcgcggcctcgagtgtcgccgcagagcagcgcgctaccccgcaaatcgcttctgactctgaacaagaaaagggtacatcctccgaagaagacgcaggacccgtcgcccccgaagtcgctgaagaagaaagcagaagatacgagtatctggtgaacctcggcgtgtcgcctcaagagatgaacagggaagaatcccagagtgaagacagctcagaggaagcggtgcagcatctgagaaaagaggaagatgacacagatgacgagatagagcaaccaagcaactag

SEQ ID NO: 37 - DNA, 333 bp, Toxoplasma gondii
gtggcggagcaagtctctgggcgttctcgccagaagcacgcaaactcctcgtctgctccgaatgcttcttctcgctcttccggtcctcgctggggagcccgagagcgattcatcgagttgccggtcgcggactggagggaagatttctacgttgcgcgagcgcagtggatctctctggaggagtggagtgacctcgttgtgctcaagctcctgtctactgaagaagtgaaaaccatgcagaacggtattcgccgccgcgcgctcgaacgagaaggagcatcgagcgctagcggagaagacggggtaggtttcgatgactctcgacttggaacg

SEQ ID NO: 38 - DNA, 450 bp, Toxoplasma gondii
gttccggcggactgcatcgaatcgtggattcctggtggccaggccaccggcgtgtacgaccacagtgcctactgggcggcagaggcgaagcagtttgagtgctacatgaagtttttgcaggaagaatttattgggaagtcacagcagcagagaatggtcgaggcgcgggcctttcaggaggcgcaggccgctgggaagcagggcgacgaagcagtggaagctgcgcgagccgcagcgaaagacgacaagaaaggcggcagtggcgccgcagactcagacactttactcgagaaagaggagggagaagaagacgttcaggacgaggaagacgaggagtcgaacgccgacctcaacgaagacgaagaaaagaggagaatgttgcaagaggctctgaaaaaagaacaaggcggccacatggccgacacccagtctacgggggcggggcggtag

SEQ ID NO: 39 - DNA, 396 bp, Toxoplasma gondii
tgggcgctggtgcccgtgcgtccgggcgacgcggcggctggagcgacgccggcggacgcagacagcttgcacagtccagagggaacgatcatgggaggtctgttctcgacgcggaagccgaaagacgcaggcgcgggcatttcaagtgctctcaagtctgtcgggaagggtgtagccgcaggcgccgcttctctcttcgttctccccgctgtcggcgcctctcaacagggcgttggaggctttttcaaaggcgtcggcgccggagtcgtcgctgctgtcgccctgcccgtcacgggtgtctgcgtcgcaggctaccaagtcgcccgcggcttcgtcaacaccccagaggcgattcacgaacgcagtcaaggaaagaaatgggacaagaaagccagg

SEQ ID NO: 40 - DNA, 228 bp, Toxoplasma gondii
ttgactgcgtcggcggctttcctgctgggaggcacagtggggaatttcgacgtttacgacatcgtggcgaatcaccgtgtttttgcaggcctttatttcggtcccctcttcatcatgttctcttttctctgtttcaacatcctcatcgctctcatcctcaagatctacgagttgtgcgcggaggaggtggagaaggcgatgaaaaagagaaaaaagcaggagagtgag

SEQ ID NO: 41 - DNA, 360 bp, Toxoplasma gondii
ggcgggtcgcgacctttcgtggtgaactcctgggcgacgttgcctctgccgtactcggttcgtttgcgagtgcatctctcctctgcctcgacgtaccctacagagatgcaatttgttgccgcgacgtcccacacattcgacttgtttctcgacgcgtcctggacagaggaggcactcctcactgcgctccagtcgttggagagtcagaaaatcgttcgcagcgtgtgcaactcgctggagatttccttcctcttctacagcaacttctcgtcgaggaaccagctctccctcacctcgctggtgtttgaaatctcctctggaggcaccgtcaaaacgactctcaacgttccgagcatggtg

SEQ ID NO: 42 - DNA, 204 bp, Toxoplasma gondii
tgcgccgtgctcatgttccagaaggagtttggggagcgactcatggctcagccgggagacaagaactactgtcgactggcggcgaacgtcagcctgttttgtacagtccagcgggtgtgcaaagtcgacgccaagcatttcacgccgcctcccaaagtcgactcggtcgtggtcaaagtcgttccgcgacccaatctcatcgat

SEQ ID NO: 43 - DNA, 462 bp, Toxoplasma gondii
gggtatgccataggctctgcagcgctggtgtctttggcgctttttggcgcctttactgtgcgcgcacacatcaccgcagtcgatgtccttgacccgtggacgtttacggggcttctctttggtgcgatgatgccgtacgcgtttagcgcgatgaccatgaagagtgtcggaatcgccgccagcgatatggtccaggaatgccttcagcagtttccactgattatccagggaaacatcgagccgcagtacaagcgctgcattgaaatctcgacacgcgcatcccttcatgaaatgattgcacccggcgccttggtcatttgcgcgcccgtcgcggctgggatgatgttcgggaaaaactgcactgccggtttgcttgctggagcgcttgtgtccggcattcaacttgccatctctgcgtccacctcaggcagtgcttgggacaacgccaagaagtacattgaa

SEQ ID NO: 44 - DNA, 270 bp, Toxoplasma gondii
ggctggcgcgtctcattcgccttcgtcggctgcctctcggtggccttcggcattgccctctcctggctgctttctccgtctccgtcttcttctctgtcttcttcgacttctccgtctcggtctgcttccctctcgtcttcgcctgcgtgtctgccaaagacagtctgtggagcggaaggctgcatgcagaaggcaaaaaggcatgcagccgcgcttttgagtctcggctacgtctttcggaccccgagcttcggtgtcatgctcctcctc

SEQ ID NO: 45 - DNA, 396 bp, Toxoplasma gondii
ggagtgttgaacgggatgccgaggagcgcgttgaatttcatcgtgatgtttttccagtactgcggcttggcggactggcaagcctcgttcactgtctccgcgagctggatagcggcgatgttggtcgcgcccgtggtggggcgtctcggagacaaggtgcaccgcttgtatccaaataaagggcggcctgtgctggcccagctggcgattctgacgcgcgcgcttctcatgtttctcgtcctctccgtagtgccgaagcgcgcgagcagctttccacttttcctcggtctctccaccctcatcggcttcatggcagggtggccgggggtcggcgtcaacaggccggttttgacggagatcgttctgccgagacaccgcgcaacagtcttcagtctg

SEQ ID NO: 46 - DNA, 231 bp, Toxoplasma gondii
gataatgtagaatacgctctgcacatctgccacagctgccgcctcctgacctcgcgtacgcgcatcttccacgcggccttggagttcagcggccgaaaagccaagcgcgaaggtgtgatgctttacgagctttttcgcaaggcgaggaggctgaagcgaagtgacgaacacatttgcctcgttgtcacgggtccgaatctgcggacgttcttgaaccatccagatctccag

SEQ ID NO: 47 - DNA, 393 bp, Toxoplasma gondii
gctctccagttgaagctcctgcagttgacggagaccgaggcagaggcgaagagtttggaggaacgcgagagagaggcgcagctcgcgcagctgcagcgggagatcgacgcgctccagaacgacttgcagggtgtacagaccgagcggaaggaactggtggctgcagttcagcagcagctgaggcagcgagaagaaatgcaggagaaaatcaaggaacttgaagctcgactcgcggtcgcctccaggccgtcgctggctgagagcgacgacgcggctgaagacgcgagacagcgaccgacaggggcggcaggagacagcgcaggagccacgaaagacgcagatgacggaggcgcctctcaagacggactccgactctccaaactcatcgaacag

SEQ ID NO: 48 - DNA, 204 bp, Toxoplasma gondii
acactaccgtcgatggctatgcatacggtccacatcctcgggtgtatcggctgctgcgtgctctccgtctgtctctaccctcagtgtatttcgtcagatgggtacgcgcctcccttccctccccggcaactgcggcgaactgtgattcactctaaaaagagaccatgccaaaacacgatgctgtctcacagaagcactctggga

SEQ ID NO: 49 - DNA, 228 bp, Toxoplasma gondii
ctcaacgaacgcgtgaacaacaacgccctcttggtgactggccaagttcagcttctcccgctgctgcggccagaaggtcttcggtgtagcccgcaggtcgtgcggtgttatctcaggtgtcttcagcaacagtactgccccagtaatccttaccacaatcaactgcatgcagctatggtttctcattgctgcttgatcattgtaaacgaagttctgccctccaaacaa

SEQ ID NO: 50 - DNA, 381 bp, Toxoplasma gondii
atgaaggggtcaacgagcacagcgctgcccgccacgccgggtgtcgcgggtaccgtgagcgtggcgggctccgccgtttcggcttccggaaaaacgaatctgccctggaatattgggtttgttgccgtgggaaatctcaagtcaaagacggtccttgatacattctacaaccggctgacgtcgaaggagaagaatgcgctcgccccgcttttccctcaagttctgaaaaccgcccccgacgctatgcctgggctgcgcagaaagttccctgtagatgatggaggaatcatgtttcttgcttccgacccaacgggatcttttctatttggactttacacccacgacaagcagtatccagagcgggtagctttcacttttctc

SEQ ID NO: 51 - DNA, 360 bp, Toxoplasma gondii
atgggtctcctacaccttctcaagggactctttcccctttgtatagctttcggaatgctgctggagacatggcgcgtacctgaaacagctaattggagccgattttccctcgacatgtatccgttgatggtgctggcggcaaaacagaatttatattctgttcttggtgtgaagcgcaacgccagtgccgacgaaataaaaaaggcatacaggaagctatcaatgaaataccaccctgataagaataaggaacccaatgctgaagcaaagttcaaggaaatttcttttgcgtatgaaatactgaacaacgcggagaaacgtcaggtctacgacgaatatggtgaagagggacttgaaagg

SEQ ID NO: 52 - DNA, 399 bp, Toxoplasma gondii
atgatgggcgttcctccggcgacaggcggcagcctggcctctggcgaagcgcgaagtttgccgagtccatccagctcttcgttttcttcgtccttcgcttcttcggcctcagcttcagagcctgctgctgcttcgagcgccgcgagtgcgtctggccagcagggcgctgccgctgcgcagaccaccggaggccagtcttcactgtcggcctccgtcgcctcttcgcttcccgacaaagatcatgtttgccagcttctcctcgacctctgcgtcctggagaaacgcgaagcggctcttgcagacctctcgaaaagacgcgagcagtaccccgacctcgctcccctgttgtggcacagcttcggcaccattgccgcgattctgcaggaaatcatcgccatc

SEQ ID NO: 53 - DNA, 201 bp, Toxoplasma gondii
gctgtacaagttggaattctcggagtgatgttttactacacatacatgcagcagtatgtgccgaataccgccgacagcgaccagagtccactgaccgctgcgtttctgcttgcctcgactctgtcgtcgacggatccggtcgcggtcttgtctgttctgaatgccgtaaacgcttcagacaaactgtgcacgatgttcgat

SEQ ID NO: 54 - DNA, 396 bp, Toxoplasma gondii
atgacgctcaagatccgtatgatttgtctcgccgccgtcggggtgctgattgggggagtcgcaataacaacagttttcaagccagtaagcccggagtgtaccgtgcattctttcagcataggccttatatctgccgacgcacttcggataagggcttcacactcaagttccaaaggtgaagaaggtgacggggaaaagcacaaagacaagagctctgaagaaggcgcaggcgaccgtgacagggaggaggaaagagatggagaaggagaaggaagtgaaaaaggacctcagccttccccgtcagggaagccgctgcagcggcgggacgctggttattggaaaaaaaactcggatagtggcaagcatgaacgtgtgaaaggtcgaaaaagaaagtag

SEQ ID NO: 55 - DNA, 231 bp, Toxoplasma gondii
ctcctcactgtccaaattgacgccgcgatcaatccaggcaattcgggcgggccggcgcttgttgacggccgtgtcgttggcgtcgccttccagggcttcagccacctgcagaatgttggctacatcgttccctacccgattattgagcacttcttgaacgatttggttcttcacggaagatacaccgggttcccctctctcggggtgaaggtctcgcacatggagaatgac

SEQ ID NO: 56 - DNA, 318 bp, Toxoplasma gondii
gtcgcggtgctgctgctgcggaagaggtacgcgccagatcctccacaggatctctgggagtcatatatcagcccggagtggcaactggctccgaatgaagagccgctgattctctttagggatctccttcgacagatatcccagcgtttccagtccgagctgaacgagctggagaacactcgcgtaggcgttcaggcccgggctatccgcgctcactaccacttggctactgatgaagaaaggcagggagacatcgcgttttttgatgagctcaagaccatgctggatagccccgagactgacgtcgcttctgtacag

SEQ ID NO: 57 - DNA, 297 bp, Toxoplasma gondii
tatctgcacctgacagccttccattccttcccaggcgactcagaaacagaggcactgttcagtgctgtcgacttctcgaaatcttcaaacacgccttgcaaaccgatgatttggcttgtcgtatgtgtagccattttgtgcttggtggctatgattgtggctctcgtgtgcggctcgtccgttctgtggaaacagccacctcaaagcacccatggagactgggccgcggcgcctatcgcagcttcacaaggaggggtgccaacggtcgtgcagctgggagccatcgactcagcctaa

SEQ ID NO: 58 - DNA, 426 bp, Toxoplasma gondii
atgtacccgcttctctgtgtttttttcgcttttttctcgcccgtgcatgcgcggagtgcttcgcagtttagtggttcccgttgtcgtccatctgctgcaattctcgtccccgccgtgttgccccgtgagcctaccaattcttgctcaggatttttgcgccaaagctacaaggacctcttgcggtgcactgtgtctccagcatctgcaccgcggagtggagacggaacgggtcatctacacataccatgttcgcgcacaccctacccgggaaacagccattaccttgcaactgtcacgcacccagaaaccgtccctgaaacccgcattttcgtcaggcgcggtctaagtctcgtatcacgtgccaagagctacctgtctccactttgtggaggccagcaagtcgatggtttcggtcgtcctccccct

SEQ ID NO: 59 - DNA, 366 bp, Toxoplasma gondii
atgaagctcgtacgcttcttgatgcggctcgcgaacgagactctggtgatcgagctgaagaacggaactgtgattcatggcacagtcgtaggcgttgacatcagcatgaacacccacttgaagaatgtgaagatgacgatgaagcaggggaacccgacgtctctcgagcacctgacgattcgaggaaataacattcgctacttcattctcccggactcgctcccgctggataccctcctcatcgacgatactcctctgcagagacctgcgagagaggcaggcagacctatccagcgactccgcggcagaggacgcggccgcggcggcagcgccttcaggcctcgcgggggcttgggccgcagataa

SEQ ID NO: 60 - DNA, 414 bp, Toxoplasma gondii
ttgatctataacttcacgagcttcaacggtcgctgcgtgagcccggttatgtaccctgactacaagctgcaagaggcgaaacagcgacagccttacgtcattcgctatggatatggaggctgcgaatacaacctaggatctttggcgtacccccgtgcctccttcggaacatccgctggtgacaaggggtggccagtggacctcgttcccaacggcagtgctggatctgccgcgacgagctgggactctccgaacgctcgcgttctgcgtcatttgggaggtctggaaacgaactacattcgcgagtacagcgagacgttcaggctgttcacgtccatgtacatgcacggaggctggctgcacattcttatcaacctttcttgccagattcaaattctgtggatcatcgaacca

SEQ ID NO: 61 - DNA, 204 bp, Toxoplasma gondii
gcgtactggttgattttgttcctttacctgctcgatccctcactgtacaagagttactctccgccgggccagctgaagttcagtggctggctctattgcaagtgtggaacgatcgtataccaggcgccacagacatacggcaacctggggcgtttctggtgctttggaagcgagaaggatgcgcagtattatcttgaaccgtaa

SEQ ID NO: 62 - DNA, 447 bp, Toxoplasma gondii
gtatttgttcgtctcagcaccgcatcaccaattctttttctgaatatggcgcgtctacatgatgcatacagctggagaaagaagcattttctcgcgaaaagtgtgtcttcgagctgcgtgtcgccgtcatgctgtctttctgggcgtgcttcttcgatgacaaccgtcagccgtgatcaacatcagagaggctttcgcatgcacacgaagaatgcaagcgagcactcgagtgccgcggaccgaaagccagatggtgctgtctcacgggctgatgctacaggctcgggctcctgcgacgaggatattgtgagatcaggcaacaacgcgaattgtgcaaaggtccccccgcctgttggctggagatggtggggcttctgtagctctataattctcctttgtcacttcttaggtcccctgttttttggctgttacgttacatggacttag

SEQ ID NO: 63 - DNA, 249 bp, Toxoplasma gondii
atggtgcgtgtgagcgctattgtcggagctgctgcatcggtgttcgtgtgcctgtctgccggcgcttacgctgccgaaggcggcgacaaccagtcgagcgccgtctcagatcgggcgtctctccttggtttgctgagtggagggacagggcagggattaggaatcggagaatctgtagagctggagatgatggggaacacgtatcgtgtggagagacccacaggcaacccggacttgctcaagatcgcc

SEQ ID NO: 64 - DNA, 324 bp, Toxoplasma gondii
attaaaacttcagatggatcgtacagcgaagtcggcaatgttaacatggaggaggtgattgatactatgaaaagcatgcagagggacgaggagattttctttcgtgcgttgaacaaaggcgaaacagtagaggaagcgatcgaagacgtggctcaagcagaagggcttaattcggagcaaaccctgcaactggaagatgcagtgagcgcggtggcgtctgttgttcaagacgagatgaacgtgatcgacgatgtgcagcagcttgaaaaggacaaacaacagcttaaggatgacatagggttcttaacaggagagagagagtaa

SEQ ID NO: 65 - DNA, 339 bp, Toxoplasma gondii
atggcggtgctcgatgtccctcttctcttcgcgttccatttcgcgttggccctctctggggtgctgcatctttctgtctcaaactcaaccgtctccgctcgtcaactgtctccttccttcccccccacagtgctgcctacctcgcgggtagcctcgcttcccgccagcgcgggcgctcctgccttcgtcaagccgagttcgatccacgcgtcagctctctcctcgtctttctcttctccctcctcttcttcttctacttctccttcttctccttcttcgtctgttccacaggcagcgctgtatcaggtcggaagcgcgttggatgccgcggcgtcgcat

SEQ ID NO: 66 - DNA, 243 bp, Toxoplasma gondii
ggatccttcctggagtactgcgacaactcgatggtgatgaatgaggcgttcgtgtacgagcgtatagactcgtatcgccggctctgggtgcatgacaagaaggagatgaccgccgcgtacctcggcagtctcctcttcgcctcaggtcatgcatgcgtggaagacatgttctacggcttgatggcacacacgctgcccgtctccaaatacgccgttgacgcctccgtcatcgactatctggtc

SEQ ID NO: 67 - DNA, 210 bp, Toxoplasma gondii
atgcagaagatgcgtcgcgacccggtgaccggccaattgccctacaagaatttctgcgatgccgtggtcaaaatcacccgacgtgaaggcatcatgtcactctacactggctaccccacctattacgtacgtatagctccgcatgcaatgatcacgttgatttcgatggagtacctgaacaagatgtggaacaggtacacgagtgtgtag

SEQ ID NO: 68 - DNA, 210 bp, Toxoplasma gondii
ggagtgacggaaatctttgtgttgatcaagggagacgaggaaggctggatggtcaaaagagttgtggaggagcacgaggaaacttttcagaaacgcctgcgccgggtcgtcaaacgcggccttcgcgtcacgtgtgtcgaggtgcctacgacggtcgactccatgggggatgccctcagagacttcagcagcaaatacgacctcagacat

SEQ ID NO: 69 - DNA, 438 bp, Toxoplasma gondii
gtgaaggttttctcgcgcgcgcagccggaggacaaaattgcgatcgtcgaggcgctgaaacgccaggggcacaccgtggcgatgactggcgacggcgtgaacgacgcgcctgcgctgaaggccgcagacatcggcgtcgccatgggcattgctggcaccgacgtcgcgaaaggcgccagcgaaatggttctgctcgacgacaacttcgtgacgatcgtcgcggcggtcgaggagggccggaagatttactcgaacatccagaaattcgtttgcttcctcctcggcacgaacatcggcgagatcatctacctgacaatcgcaatcgcagcctcgatgccgcttcccttggaggccctccaggtgctcttcctcaatctcatgagcgacggctgcccagccgtcgctctcgccaaagaaccatcggacgacgaaaatatg

SEQ ID NO: 70 - DNA, 384 bp, Toxoplasma gondii
atgcgactacagatcgtcgcgacaacagcgcttttcttgggtcgagtccaggtgttcgccgtcccgttcgccggtgcaccactcgcctctcggcctattgtgcctcgcgcgcagagtcacttttttgtgtgtgcatctcccgagtcgaagattttccgccgaaagaactcggaaaacggcccacattcgcgaatcttcacagccagcgaagtcgcactcgtcgagcggcgctccggtgtcggtacacccgccaaaaggccgtccttgtcacgaggtcaccatgaggatttctctgaagaagcgcaatgggaagttcatcgacgcgttcgagcttcccgaggactgcacagtggaagaattcaagcaactcttctaccaaaagtt

SEQ ID NO: 71 - DNA, 231 bp, Toxoplasma gondii
gtcgccttctggctgacgatgttccacttcatgaagcgggagctggagacgctcttcgtccacagattcagcagcagcacgatgccgattgtgaatgttcccatcaactgtgggcactactggattctctttggtgtcttcgttggatattttctcttccacccgaaataccagccgatgtggcgagacgaccagcctctcgtcatctacagcctcgcggcaaccatggtg

SEQ ID NO: 72 - DNA, 390 bp, Toxoplasma gondii
atgctgaccagttggctgagcgtacgatgttgttctctcatccacattggcggcggcaggactactagacaaagtttctcctacatgagccactttatgagacttttttgtcgccggaaactttccttcctgacttcattctcaggtcacagtccaacccggagtgacatacaggttgcgccactacgcactgagtcttgcgtagcagcaacgcgaggagcccgtccgcatcacttcctaccccgagccactcactgttcttcggcctctagcacaatggtggctgctggagtttctcatggaaaccgttcgctgacttactttccagcgagaagagacgaaagtgttgttgaaaggcacttcggcgagaccgttgcggatccctacaga

SEQ ID NO: 73 - DNA, 363 bp, Toxoplasma gondii
atggcgtctgtaaaacgcgtcgttgtggcggtaatgatcgtgaacgtgctggctttaatttttgtgggcgttgccggttcaacgcgtgacacagggtcaggcggggatgactccgaaggtgcttgggggggtgaacaacaacaggtacaacaacacggacaaagtgaagaccgatcgttattcgaaaggggaagagcagcggtgactggacatccagtgaggactgcagtgggacttgctgcagctgtggtggccgttgtgtcactactgcgattgttgagaaggaggaggagacgcgcgattcaagaagagagcaaggagtctgcaaccgcggaagaggaagaagttgccgaggaagagtaa

SEQ ID NO: 74 - DNA, 402 bp, Toxoplasma gondii
atgcgccagtggttggcgatctcgtcgcacaaagacattccgccgctgctccttctttggtgccgctgcataagtctcactcactcaccaattccaccgcctgtcgtcctcgccgccgcgccgcctccgggagtagcgacgccccagccggtctccgaagacgccggggcgagtgcggtcgccgccgccgtacccgagacgcatgcggcggagagcgcgcgcggcgcgtcgaaggcggaaaacgcgcaggagctggcggggggaactgcggaagccgcagcggagagcagggggcaggcggtcgccaccgagggtggtgtgcgtacacccgacgcagcggcggactcgacggagcggagggaggaagacgaggcagaggacgaacagtataggcggcagtcg

SEQ ID NO: 75 - DNA, 414 bp, Toxoplasma gondii
gcgcgattcgtcgttgtcagtgcttgcggtcagtcgctgctgttggcccagcgtatgggctacggactgaacttcagctgtctcccgatggccggaagcttctactttgcgcccgaaatgttgaatgggaaagtctacacgtgccaagacccgcggctcccgttcgctgcggtgcacggagatccagatctcgttgcagtgggaaagacgaggttcggccctaccgcgcttccgctccccatgctcgaacgctacaacatgtcttccatcggcgacttcttgcgtgttattaaccccgacataaacctcgttaaggtgtatttggatctccttggcacgtcccacatgcgcaactacgtcttgagaaacttcctcttcgaggtcccgaagctgaacaccgtgctcttcgccaac

SEQ ID NO: 76 - DNA, 354 bp, Toxoplasma gondii
gacctggaagtggcgattggcctctcggcgatggttccagacaaggtggcggcgcaccttctgcacgtcgtgtacagacacaacagtggcgatccgacgcagatgcatgcctctcacacagtccagacgggagagaagttcgacgaagacggcgtcttgagcaaagccgctgtgacactccaggaaatcatttcgggagcagcgcgcacggtcgcagccgccacgcgcgcgttgacggagaaggacgagcgcgagaagcaccgacaaactccaagagccttcacagagagtcagccgacaaacaagaaaatcagtttccaggaggctctcggcaacaccacgcgcttcggcatc

SEQ ID NO: 77 - DNA, 216 bp, Toxoplasma gondii
gggggacatcggcaactgcttttggtggcgatcccgaggcacctgtacctctttgtggggaatccgacgctctctgcagtatttcagaatctttcttcgtcggatcttaaaactttcttggcatttgaagtgccccaagaaagtccacaagcaggccccgtgctccagctgctgaagaagaaaggcagcgtggacgtgctcgttttctggaccacg

SEQ ID NO: 78 - DNA, 201 bp, Toxoplasma gondii
gcgctgatgggaagcagtggagcaggcaaaacgactcttctgaatgttctgtcggggcgcgtgacgaagaatgtcggaggccgcgttcagtacaacggtctggaattgcctcctgaggcactgaaggcaatttcttgcttcgttcaacaagaagtgattttcttcggaaccttgaccgtgcaggagcatctcgagtaccag

SEQ ID NO: 79 - DNA, 201 bp, Toxoplasma gondii
tacagcgtcgattttaccatccacgtcgcgcataccttcacacactgcgtcggcgcgagtagaaaagatcggatggtcgagacaatgattgtcatgggcgcgcccgtgactcatggaatgctgtccaccctgttgtcgattctcgcacttgcggggtcaccgaaatacattctagaagttttcttcaaaatgatgttcatg

SEQ ID NO: 80 - DNA, 225 bp, Toxoplasma gondii
caccgcattctccaggacgccgtcttccttgcgacagattctccgcagacgggaggcattttgacggtgaacaagcggggactggtttgcctgtgcaacatcaacctgcaagcgctcattccgtacatcaaccaggccctcgtctacgtcccgaatcgccagcagattgccaccagtctggcgaaaagatacggtctcccgggagcagaagaaactctcatgcag

SEQ ID NO: 81 - DNA, 426 bp, Toxoplasma gondii
atggcgcaggacgcgactgcggcgcctccagctggcgcagccggcgcagccgagaagaagagcgcggcttcttcgcctctgctcactctctgcatcgccgtcgaacagctgcggcagggcgtcgagcagcaagacgatcgccaagtcacgcgcatgtttcgccagttcaagactcttcgcacaacttgcactccacacactctgctcctcgttgcttcgcgcctcctctggaactccgatcccggcaacgcgaaggcgaacgcagaacctgaacaaggcgcaggctcctccgctccagcgacgactgcagacgccgcgcggtcgactcgcgacgaggcgacgctcgccttctggaaggccatgcaacgcgctctccacgccgcgggcgccgaggacggaacccaggcgatggacgtcgacattccg

SEQ ID NO: 82 - DNA, 231 bp, Toxoplasma gondii
tggcatttggaggcgtttgacctggccagaacggttgtgtcggagagctgcaccaagttacggagcgaagtgaaacgcgcagttggagatggacgagcgttgctttctgcaatctgtgcgctgatttgtgaactgcttcgtagccctgtaaagtctttccaggtgagaggaacaaggaccaccacggcgcccctcgaacaacgcatggacgccgtttgtgtgaacgaatga

SEQ ID NO: 83 - DNA, 465 bp, Toxoplasma gondii
gcttcgcgtgcgtcggcaggtcggtcctcgtcctgctcggcgtcgcaggtgttcgcctcgctggtcctcacgccccaggacatgtgcagttctccctcgttttggcgactctttctgatgttgttgctttcctggcagtcgctcttcttcgtccagctgttctggaaagtcctgccgctctatccggaggcttccctcgccgcctcagccgctgcgccagcgcctctggactcgctcttcgcgtcggtcgtgcgccgagtcccgcgcgccctcgcctcggcttcgcgtccggaccctcgggcgcttcatgcagctgctgacgcctggagcctcactgggtcctcgtggagcttcgcaaacgacttctttctcagctgtctaggcggccttctcggcgcactctgctgcttcggacgccttctgtggggatacatcggaggcggaattggatatatgcggagcacg

SEQ ID NO: 84 - DNA, 333 bp, Toxoplasma gondii
tctatcgtgattgggacatacacaggagtcggctcttccgtcgttctcttcagtcaactctcgttcggggccttggggtacggtctagcagtagccatcgttctttacctctggctcttgacagtccgcgaattcgagattgttcaagtgactggaacaattgcggccgtcttcacggtcttcttcacagcggaccacttcctccacgtcagcggtgtcctggcggttgtcactctcgggatttttatggcggccaagggctcgacagctctccagaggtccattcagaaactccaccatgagtctgtcgagcttctttccacgctttcggtc

SEQ ID NO: 85 - DNA, 312 bp, Toxoplasma gondii
tggaaggagtgtttcatcatctcgtacggtggccttcgcggcgtcatctgtctcgccttaggcctggttgtcgaggcagatcctctcatctccccaacgctgcgggagcacgtcggaatctgcgtggcaggcactgtgatctttacgcttctcataaatggaacgacagcagagctggcgtacacgcgtctgcggctgtaccccatctccaagtatcgccgagagtatctggattcggtgttggcctccattgatgtctcgttcaaccgcaagaaaaacgaactgaaggactactggctgttcagaggaaca

SEQ ID NO: 86 - DNA, 357 bp, Toxoplasma gondii
gagcaagcacaggtggcgctgaatatccaggcagacctgaccagttgcttcaattggaacacgcagcagcttttcgtttacgtcattgtcaggtacgagacgccgaagaacccgcgcaacgaagtgattgtctgggatcgcatcatcaccgaccccgatgatgccataatagatttcgagggcgttataaacaagtaccctctgagagacaacggccgcagtctccgaaaccgcacggtcactgtcgctctcgagtacgcgtatcacccggttgtcggggtcatcaaaagtggccacgtcgcctcttccacgtacactctgccgtcctcgtattttcggtatgccaaaggcaactaa

SEQ ID NO: 87 - DNA, 204 bp, Toxoplasma gondii
ttcttgcctgcggtgtcgaaacttgttcgctcagtgctgccgatgacgcacaaggaacgatttgaaaagattggcattcgacctccgaagggtgagtttcgatgcttcttccgcgtcgccgttctcccgactcggctcctcgttctctgtgtcgtttcctctctctctcctcgcaccctcccgtcgtcttgcgactctcttcca

SEQ ID NO: 88 - DNA, 234 bp, Toxoplasma gondii
ggatacatcgcgcctgagcagagaacagctgcgtacgacagaccagcggacgtctgggcgttcggcgtcgtctgcgcgagactgcttgggctgagagaatggaagaaactgagcgaccctcagcaccgagttgctctctccgacttcgctctcaaagacgcgttcctcatcaacctgtgcctcgcctgcctcgaacggaggcccctcatgcgcccgaccttccaccaaattgtc

SEQ ID NO: 89 - DNA, 228 bp, Toxoplasma gondii
gatttgctgatggatgagcactttcttttgatcatgaagctgaggattcaggagcgaccgcctcgctcgcggagagaaaaagacattttcgtcatcgtcaacaagttcacagtctccctctacgaagacgttgagtccctcgctctgcagaacgagaaggagcagctgaaaaaaattcgccttctctgcatgcaggctctcgtagacatcgacaaactgacggaatac

SEQ ID NO: 90 - DNA, 300 bp, Toxoplasma gondii
gttgtgattatttacaagcacttctacaaggagacgtgggacatcgcaagcgaggcactgatgatcccagctggacacaatccgcaactcggtcacctcctcgtcgccgtgatgctaaatctttctgtccacgtccagagaagctgcgcgtatcgcctcaccgaactcgggcctcaagtggaagcccgattgcggtcggaagatcagcggacagttcgacttgtaaaaaaccgggatctcaacgttcctttgcggaagttcgctgagattcgtctcattctcgaacgaggctcaaagaca

SEQ ID NO: 91 - DNA, 348 bp, Toxoplasma gondii
accattgttctcggcgaaatcttccaaaagtgcccacgaaagcatgttttttcggccaaccttccttccttcaatccgtctattatgaagcatctaatctcgctgtacaataccgtctcgttgggtcctagccaagggtctgagacaggtagacgacctggagtgtggagtgcctgcagcgtcttcaagcatgaggtggccgtgtgcaagctgcaagcgattttgaaggatctgggaccgattgtttccggagagctcatcgctacctccatgatgcagaatgtcactgttaccagcgccggcagtttcggcggcaaacgcgcgcctcgcacagcagcacaacaacga

SEQ ID NO: 92 - DNA, 222 bp, Toxoplasma gondii
acgctatgcggccaaatgcattttcagaggggcgctaacgtagctgtgacggtagatctcctcagcaaaacgaatatgttttccttcaccatatggggcattaaaggctttggccgtctgacttgggacgaagaaaagaaagcgtttgaggtgcaaaggtatcttcatcactatgagcatcctactctccaccctgtgactggaccgacatggtcagttaag

SEQ ID NO: 93 - DNA, 222 bp, Toxoplasma gondii
gtgacgcgtacaatcctcacgagcacggcattttcttgtgcagtctatttcttctttggctatctctgtgcggcggccttccccgctccctcggcctcctcgaatgtcatcgccgccatgcttccctcctctccatccttcgtcgctgtcgcatgcatctgcttcttccacgttttcgccctcctgccgaacatcgtcacggggcaaatcagttgccggtga

SEQ ID NO: 94 - DNA, 231 bp, Toxoplasma gondii
ggaagtctttggcattccgcagttcttggcgcacccgcgagtgctgcgccaaatacgattttcgaactcgcaccgacagcagcagaagctggaagagcaattgcttttcgcccgcgtctggatgcatactatgacacggcagctcacaagatcgttatgcttttcgaccttccgggcttcgagaagaaggatatttctgtcgaggtcacggaccacgccatcatcatttcg

SEQ ID NO: 95 - DNA, 204 bp, Toxoplasma gondii
ggtctcgacgttcttctttggctggtttgttttttcatcacggtcgtgttcggggcgatggaggggattctcgcctcgattgttctttctcttctctggctgttgcgcaaaacagcgcggccgcagtgcatcgtcctggggcgccttccacaaacttacatctaccgaaacatcgagcgttttcgcatggcaaaagaggaacca

SEQ ID NO: 96 - DNA, 201 bp, Toxoplasma gondii
tacgtcgtcgatgtcctcggttcagaaggacccggctcgctggtcgcacacctgcggaaacaaggccttgcagagtcgatcgacgttgaggccgaagaaagccgctgctactcgactcttcgcgcctccgtggaactcaaagacttcaacatgaatcacacggccatcatgctgatcggctcttctctcttctcctatcta

SEQ ID NO: 97 - DNA, 459 bp, Toxoplasma gondii
aaactcgtaaggaccattctattctgccatggtcgagtgacagtggcaagtcgcgaggcatggctctttctcggtctgctcctcgttctcgcgctctgcgcgtccgcctacgtcctctgcgaggggatccaaaacgctgagagatctcgcttcaagctctttctctcctgctctcacatcgttatggcggtcgtgcccgccgaattccccatcaccctctcgctcgcagtcaccatggcgctcctctttctgttcacgcaacaaatcttctgcacagagccgtttcgagtccccctcgccggccaagtcgatgtatgtgccttcgacaaaaccggaacgctgacctctgactccatgcgcgtcaagggcgtctacggtgtacgtacatccgagggaaaggccgcatcctctcggcctggcgagcgcgcgggcgacgaagaagaaactctcgtcacgcag

SEQ ID NO: 98 - DNA, 444 bp, Toxoplasma gondii; identical to SEQ ID NO: 11

SEQ ID NO: 99 - DNA, 1278 bp, Toxoplasma gondii
atgggcaagacattgctggggaaagtcaaaagagcaacagggatgggagtgggagaaggtccttcagttgccaaaaagccgaaatacacagcgacggtcccaggattcactccaccgtctggcgatcagcggatgaatgaattcatggctgtggacacatcaggggaattcatgcgtcatctctacatcgaagaagggcgcacagtgtgtgctagtgccacctcccgcaatagaagaccgacgagcgagagcccccacagcgatgatgtggtggtcgtcgaaggaatgctccggggaagaccagaaacgcgagtacatgccatgttcgacggattccagggacgccacagtgctatgtggctcgcgcagaatgtgatgaactatctcaacgatctgagagatgtcaacgaagaggagattactcggcagttcgagagaatggatggagacttaagagcagcgaatctccctggcgggtcgtcagctctaatcatttttgtacggtatgaaaaaaaaccgactgaggcccgggtggtcggtaggcagatagtaccggaaggtgccaaggagttcacgtcagtcgctgaggcgctaggaggtccgctcatgccagtggtagccatgaacttcaggcgcgatcccagagctgccaaggggatctataccatacatgtcgctagcttaggcaactctcgttgtgtacttaagtctggcagaactgcgattcacttgtcgactcctcacactgcctcgtctcataaggaaagacaccgcgtccaggcagcaggaggggtattcactacagtcaatggcgagcttttgctaggcggagttgtccctatgacgagggcattcggcagttttgacttcaagaaaggaggacaagggaaacttcaacaggatttggtgtctgcagttccagacgtaacgacgttttttgcatatcctggtgacgatatcgtcgcgggtactgcaggcgcattcgctcatttcagatcacatgcggcaatagcggctgcgatcgctctctacccggtcagtccagaaacagttcttgacgcagcaaaagcaatggtggtgaatgcaaaaaggaggaaggtaaccaaaaacatcagcaccttcgtaaggcatctccccgagtcacgcacaaggagtcaaaagatgctggagggcacctccggagagaatggcgaagaagacttctcgatcgacaggacaaacgagcttacacaggcgcttcaggcgggatttttttccttcttattgtattatccatga

SEQ ID NO: 100 - DNA, 3699 bp, Toxoplasma gondii
atgtccaccagcggtcagaatgcctccccccgcccgacgaaaacagataaaattcggaaaccacgaaacgacagtagaaactaccgctacatcgagctgccaaacgagttgcgagccctcctcgtttcagatccggaatgcgatgaagcagctgcatcgatgcgtgtgggtgtgggctccatgtcggacccgcccaagatacctggcctggcccattttacagaacacatgctgtttcagggatccaagcggttccccggtacgcatgacttcttcgatttcgtccacaaccatggtggctataccaatgccttcacgagcaagttttcgacggtcttctccttctccattggccccgggttcctggaacctgggctcgaccgcttggctgacctcttctctgccccgctgctgaaaagcgaaaacctgctcaaagaagtcaacgccgttcattcggagtacatcattgacctcaccgatgacggccgtaggaagcaccatctcattcggcagacagctaaaggcggtccgttctctaatttcaccgtcggaaacctcgagagcctcatggagcggacgaagcagcagggcatcgacccggtcaaagccatgcgcgagttccacaacaaatggtactcgtcaaatctcatgacgttggctgtggtgggccgagaatctcttgatgtcctcgaaagtcacgtccgcaagcacttcggcaacgtccccaatggtcgtgtcacccctccagtattcgaagaatgttctgaagcattcattcctcttgatccgaatgaactcggtactgaaacgctcgtagttccggaagcagaccttcacgatgccactttcgtcttttatctgccaccccaagcgaagaactggcgaagcaagcctctgcagttcatttctgaaatgctggaacacgaaggaccaacatctctgtcaagcaaactcaaacgcgagggtctcatcacgtctcttgttacggattactggtcgccagagctttgcaccgtcttacaggtcaacgtgcgcttaacagaaggaggacgatccaaggagagcgtctataaaataggccacgctcttttcacctttcttcgaaacctcggcgtctctcgccctgaacgctggcgtgtgacggagatggccaagatccgccagcttggattcacgtttgccgacatgccggatccgtatgcattaactgttagagccgtggaaggactgaactactacacgcctgaggaagtgattgctggagatcggcttatctaccatttcgacccggacatcattcagcagtatgtgcagaagttcctggttcccgataacgttcgtcttttcatatttgataagaaacttgcggcagatgtggatcgcgaggagtactggttcaagatcaagcatggagttgaacccataatggattccgcgttcaagaagtggaaggaaatcacctcagccccagctaacacggtgcagagtatgatgcgaatggaaggcatggcacttcctgctccaaatcgattccttccaaataatgtcgccatccttcctcgtccgcctggttccgggcaaggcgctgagaagagtgccttcccggagcctcttgtctttgccggcgaccacatctgcgccaacaaatgtgctgtttttcacaagcaagacaccacttttcactctccaaaggctgtcgtcgagcttcaaatatattatactggaagtcatgaagacggcgtccgcgagcgcatcctgactgcgctgtacgtgcagagtcttagattcgctctgcgtgagcggttcgccgatacgcacagggcaggcatgtctcttggcctgggtagcggtgtcgccgtcgggtccaccacgcttccgaccgtaaagcgacagagagtgctgacactctctgcggcaggcttttcggacaagctcgattatgtgcttcgcgcattcgcaaagtctctcgcggcaactgagggtgcgtcagatgaaaagccaatcccttcacctatggaacttggagtcactatgtctcacggcgtgagacatttgatgcactcccgcgtcaccgctccatctaatgtactcgcttcggtccagtcaagccgtgtatacaccaccgcagttcgcttgcctggaggcggccgagccgcgcgtcggaccctgacacacgaccagatcacaactatgcatcggaaaccggcctcgatggatgtaactctgatgagaactcccgtatcctcattgggcatccggatcgtcggcggaagagcagaaagagaagctacatcggttgttgctggtggaggaaaacccctcctggagaagcgtttcttcaggctggcctttgatcagctgcggacagacttgcgagtagcaaccctcaaccgcacccctttagcgcaagcgaacgacgctgttttggagctgatcatgcacccacatgttccagtccacaaaatgtatgccgccttattcgaaatggaaaagaatcacccgactctggacgcaatcttcgaagaggttgcagactggggggtaaagctttggaatgaagctgccgtcgagggacttgtccagggtaacattacctccgagtcagccacacagctcgtaggggacgtaatttccctacttccactcaaaaacatcgtcgcggccaacacgattgccaaacccacaatttcgtcgttttcttccctgaggagagcaagttccgttgccccacaatcctcgtccaaacaagattccgttccttcatctgacatgcctacgtcgaccgagacactatccatgccgtcgtctgcggagagtcaaacgcctgaatcaacagaccgcgcctctttccctatggagtctactacaggccccgcgccttactctgtggagttgtcaacagggcgcacaccataccctgatgatcttccaacaagcagtacaccataccctgatgatcttccaacaagcagtacaccataccctgatgatcttccaacaagcagtacaccataccctgatgatcttccaacaagcagtacaccataccctgatgatcttccaacaagcagtacaccataccctgatgatcttccaacaaggagtacaccagccgctgatgatcttccagcaagcagtacaccataccctgatgatcttccaacaagcggtacaccataccctgatgattttccaacaagcagtacatcataccctgatgatcttccaacaagcagtacaccataccctgatgatcttccaacaaggagtacaccagccgctgatgatcttccaacaagcaggacaccataccctgatgatctcccaataaggagtacaccttcaccgattgtgctttcagcgggacgggcaccgtccgttgtcgtctctccaatagagcgtacatcatcccctgctctccttccaacagagcgtacacctctcactgaacgtattgcgcaggctctacggccagttggagaggcggcagctgtgaggaggctcttagtattgagaagatcaagcatccaccgaatgccctatagtcccttccagtactcgagtacctggtttcatccgagctcgaatagtcgaagacaaatgatgctgcgcatggcattaaggaggcagctgggggcactgactagacaggcgcagccagacacagaggggaagccatcgactgtcttcgtgttccgt